(19) United States
(12) Patent Application Publication
(10) Pub. No.: US 2024/0050660 A1
(43) Pub. Date: Feb. 15, 2024

(54) Method Of Using A Plungerless Aspiration And/Or Injection Device

(71) Applicant: Gholam A. Peyman, Sun City, AZ (US)

(72) Inventor: Gholam A. Peyman, Sun City, AZ (US)

(21) Appl. No.: 18/382,290

(22) Filed: Oct. 20, 2023

Related U.S. Application Data
(63) Continuation-in-part of application No. 18/229,970, filed on Aug. 3, 2023.
Application No. 18/229,970 is a continuation-in-part of application No. 17/144,075, filed on Jan. 7, 2021, now Pat. No. 11,730,890.
Application No. 17/144,075 is a continuation-in-part of application No. 16/036,826, filed on Jul. 16, 2018.
Application No. 16/036,826 is a continuation-in-part of application No. 14/742,323, filed on Jun. 17, 2015, now Pat. No. 10,022,457.
(60) Provisional application No. 62/958,101, filed on Jan. 7, 2020.

(51) Int. Cl. A61M 5/28; C12N 9/22; A61K 48/00; A61K 41/00; A61K 47/68; A61K 47/69

(52) U.S. Cl. CPC A61M 5/282; C12N 9/22; A61K 48/005; A61K 41/0028; A61K 47/6849; A61K 47/6929; C12N 2310/20

(57) ABSTRACT

A method of using a plungerless aspiration and/or injection device is disclosed herein. The method includes providing a plungerless aspiration and/or injection device; administering to a patient in need thereof, using the plungerless aspiration and/or injection device, a plurality of nanoparticles coated with a biocompatible molecule for cell uptake, the nanoparticles conjugated with at least one gene, an antibody that targets the nanoparticles to a target cell, and a deoxyribonucleic acid (DNA) editing complex forming a complex of coated nanoparticle-gene-DNA editing complex; delivering the coated nanoparticle-gene-DNA editing complex to the target cell using the nanoparticle of the complex as a carrier of the complex to the target cell without using a viral vector and without using a plasmid vector; and stimulating the coated nanoparticle-gene-DNA editing complex with one or more energy sources under conditions sufficient to introduce the at least one gene into the target cell.

### CROSS-REFERENCE TO RELATED APPLICATIONS

This patent application is a continuation-in-part of U.S. Nonprovisional patent application Ser. No. 18/229,970, entitled “Plungerless Aspiration And/Or Injection Device And Method Using The Same”, filed on Aug. 3, 2023; and U.S. Nonprovisional patent application Ser. No. 18/229,970 is a continuation-in-part of U.S. Nonprovisional patent application Ser. No. 17/144,075, entitled “Plungerless Aspiration And/Or Injection Device And Method Using The Same”, filed on Jan. 7, 2021, now U.S. Pat. No. 11,730,890; and U.S. Nonprovisional patent application Ser. No. 17/144,075 claims priority to U.S. Provisional Patent Application No. 62/958,101, entitled “Plungerless Aspiration And Injection Device And Method Using The Same”, filed on Jan. 7, 2020, the disclosure of each of which is hereby incorporated by reference as if set forth in their entirety herein.

This patent application also is a continuation-in-part of U.S. Nonprovisional patent application Ser. No. 16/036,826, entitled “Methods To Regulate Polarization And Enhance Function Of Cells”, filed on Jul. 16, 2018; and U.S. Nonprovisional patent application Ser. No. 16/036,826 is a continuation-in-part of U.S. Nonprovisional patent application Ser. No. 14/742,323, entitled “Methods To Regulate Polarization And Enhance Function Of Cells”, filed on Jun. 17, 2015, now U.S. Pat. No. 10,022,457, the disclosure of each of which is hereby incorporated by reference as if set forth in their entirety herein.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### INCORPORATION BY REFERENCE OF MATERIAL SUBMITTED ON A COMPACT DISK

Not Applicable.

### BACKGROUND OF THE INVENTION

### 1. Field of the Invention

The invention generally relates to a method of using a plungerless aspiration and/or injection device. More particularly, the invention relates to delivery of combined methods to regulate polarization and enhance function of cells using a plungerless aspiration and/or injection device.

### 2. Background

Despite progress in blood liquid biopsy, the aqueous and vitreous fluid is seldom used for diagnostic purposes except in cases of endophthalmitis. However, the aqueous, vitreous, and subretinal fluid contains many molecules that could provide information about the health of the retina, choroid, optic nerve, or the health of the lens or the trabecular meshwork of the eye.

At present, most medications are delivered to the eye by topical drops, or injection using known standard syringes. The topical application has the disadvantage of being washed from the surface of the eye rapidly by the flow of the tear film. The injected medication stays in the tissue a longer time. The syringes have been long used for either injection of medication or aspiration of fluid including a liquid biopsy from the eye or the veins, arteries, or body cavities.

The syringe is composed of a circular tubular body in which a plunger moves forward by the thumb of the user or the plunger is withdrawn using thumb and index fingers. A needle is placed on the syringe to penetrate tissue and deliver medication. The needle can be mounted on the syringe or turned in or out via a luer lock to the syringe.

The plunger also can be connected to an automated air pressure capsule for injection of mediation only. When activated, it forces the tip of the needle and medication, thereby injecting the medication in the tissue.

The use of a syringe for injection is in general cumbersome. It is hard to control the degree of the needle penetration in the tissue with the thumb, and aspiration requires the use of both hands to have the syringe stable in place. While one can predetermine the length of the needle that enters a cavity, it is still difficult to withdraw fluid without the use of both hands, one for stabilizing the syringe and another for withdrawing a liquid biopsy.

In general, the automated systems of injection for withdrawing fluid work like “one size fits all”, but fall short in practice.

Therefore, there is a need for a plungerless aspiration and/or injection device for facilitating the aspiration of a liquid biopsy or the injection of a medication in bodily tissue. Moreover, because of the sensitive structure of the eye, there is a need for a more refined aspiration of the liquid biopsy or injection of the medication in a corneal pocket created during refractive surgery, or in the small anterior chamber, under the conjunctiva, in the suprachoroidal space, or for the sub-retinal injection of a very small volume of medication. Furthermore, there is a need for using a plungerless aspiration and/or injection device in various methods to regulate polarization and enhance function of cells.

### BRIEF SUMMARY OF EMBODIMENTS OF THE INVENTION

Accordingly, the present invention is directed to a method of using a plungerless aspiration and/or injection device that substantially obviates one or more problems resulting from the limitations and deficiencies of the related art.

In accordance with one or more embodiments of the present invention, there is provided a method of using a plungerless aspiration and/or injection device, the method comprising the steps of: (i) providing a plungerless aspiration and/or injection device; (ii) administering to a patient in need thereof, using the plungerless aspiration and/or injection device, a plurality of nanoparticles coated with a biocompatible molecule for cell uptake, the nanoparticles conjugated with at least one gene, an antibody that targets the nanoparticles to a target cell, and a deoxyribonucleic acid (DNA) editing complex forming a complex of coated nanoparticle-gene-DNA editing complex; (iii) delivering the coated nanoparticle-gene-DNA editing complex to the target cell using the nanoparticle of the complex as a carrier of the complex to the target cell without using a viral vector and without using a plasmid vector; and (iv) stimulating the coated nanoparticle-gene-DNA editing complex with one or more energy sources under conditions sufficient to introduce the at least one gene into the target cell and, using the DNA editing complex, to modify a pathology-inducing DNA in the target cell to treat the patient, wherein stimulating the coated nanoparticle-gene-DNA editing complex with the one or more energy sources includes stimulating the nanoparticle of the complex with ultrasound or light.

In a further embodiment of the present invention, the DNA editing complex is selected from the group consisting of a CRISPR/cas9 complex, a zinc finger nuclease (ZFN) complex, and a transcription activator-like effector-based nucleases (TALENS) complex.

In yet a further embodiment, the coated nanoparticle-gene-DNA editing complex is further conjugated with one or more Rock inhibitors for reducing inflammation at the site of the target cell, and additionally conjugated with an activatable cell penetrating peptide (ACPP).

In still a further embodiment, the nanoparticles are directly attached to DNA containing the at least one gene.

In yet a further embodiment, the method further comprises, before administering the coated nanoparticle-gene-DNA editing complex to the patient, administering the coated nanoparticle-gene-DNA editing complex to in vitro cultured patient stem cells.

In still a further embodiment, the at least one gene of the coated nanoparticle-gene-DNA editing complex further comprises an opsin-family gene and an ion channel gene, wherein the DNA editing complex comprises a CRISPR/cas9 complex, and wherein the method further comprises the steps of: (v) administering the coated nanoparticle-gene-DNA editing complex comprising the opsin-family gene, ion channel gene, and the CRISPR/cas9 complex to a non-excitable target cell of the patient; and (vi) stimulating the non-excitable target cell of the patient with light so as to modify cell membrane polarization of the non-excitable target cell and modify the pathology-inducing DNA in the non-excitable target cell to treat the patient.

In yet a further embodiment, the DNA editing complex comprises a CRISPR/cas9 complex, and wherein gene modification is done using CRISPR/cas9 mediated homology-independent targeted integration (HITI) or homology directed repair (HDR).

In still a further embodiment, the light stimulating the nanoparticle of the complex is delivered by a fiber optic.

In yet a further embodiment, the method further comprises the step of: (v) administering a PNA/DNA/PNA complex to the patient, the PNA/DNA/PNA complex being formed from a peptide nucleic acid (PNA) and a cell penetrating peptide (CPP) or activatable cell penetrating peptide (ACPP), and the PNA/DNA/PNA complex providing therapy for a medical condition of the patient.

In still a further embodiment, the plungerless aspiration and/or injection device comprises a housing; a needle portion disposed in the housing, the needle portion configured to be selectively retracted and extended by a user, the needle portion comprising a needle tip configured to be inserted into tissue of a patient for aspiration, injection, and/or implantation; and a bulb portion disposed in the housing, the bulb portion defining a fluid containing cavity that is fluidly coupled to the needle portion, and the bulb portion being elastically deformable so that the user is able to perform the aspiration, the injection, and/or the implantation on the patient by manipulating the bulb portion.

In yet a further embodiment, the step of administering the plurality of nanoparticles using the plungerless aspiration and/or injection device further comprises the steps of: (v) filling the fluid containing cavity of the bulb portion with the plurality of nanoparticles; and (vi) compressing the bulb portion of the plungerless aspiration and/or injection device to inject the plurality of nanoparticles into tissue or a body cavity of the patient.

In still a further embodiment, the plurality of nanoparticles comprise a plurality of piezoelectric nanoparticles; and the step of compressing the bulb portion further comprises compressing the bulb portion to inject the piezoelectric nanoparticles in the vitreous cavity to migrate into a retina of an eye of the patient, or directly into the retina, in the subretinal space, or in the choroid, so that the piezoelectric nanoparticles are able to be stimulated with ultrasound from outside the eye of the patient.

In yet a further embodiment, the method further comprises the step of: (v) filling the fluid containing cavity of the bulb portion with progenitor nerve stem cells or mesenchymal cells in addition to the plurality of nanoparticles; and the step of compressing the bulb portion further comprises compressing the bulb portion to inject the progenitor nerve stem cells or mesenchymal cells into an eye of the patent so that the progenitor nerve stem cells or mesenchymal cells migrate under a retina of the eye to a central foveal area through retinal intercellular space, and replace retinal cells damaged by age-related macular degeneration or retinitis pigmentosa.

In still a further embodiment, the bulb portion is manipulated independently from the retraction and extension of the needle portion such that the needle portion is able to be extended or retracted without deforming the bulb portion.

It is to be understood that the foregoing general description and the following detailed description of the present invention are merely exemplary and explanatory in nature. As such, the foregoing general description and the following detailed description of the invention should not be construed to limit the scope of the appended claims in any sense.

### BRIEF DESCRIPTION OF THE SEVERAL VIEWS OF THE DRAWINGS

The invention will now be described, by way of example, with reference to the accompanying drawings, in which:

FIG. 1A is a side cross-sectional view of a plungerless aspiration and/or injection device, according to an illustrative embodiment of the invention, wherein the needle of the device is in a retracted position;

FIG. 1B is a partial side cross-sectional view of a distal end of the plungerless aspiration and/or injection device of FIG. 1A, wherein the needle of the device is in an extended, exposed position;

FIG. 1C is a partial side cross-sectional view of a proximal end of the plungerless aspiration and/or injection device of FIG. 1A, wherein the bulb and the fork of the device are illustrated in a compressed state;

FIG. 1D is a side cross-sectional view of an eye, according to an illustrative embodiment of the invention, wherein the plungerless aspiration and/or injection device of FIG. 1A is being used to aspirate aqueous fluid from the anterior chamber of the eye;

FIG. 1E is a side cross-sectional view of an eye, according to an illustrative embodiment of the invention, wherein the plungerless aspiration and/or injection device of FIG. 1A is being used for intracorneal cavity injection;

FIG. 2A is a side cross-sectional view of a plungerless aspiration and/or injection device, according to an illustrative embodiment of the invention, wherein the manner in which the device is used for injection is illustrated;

FIG. 2B is another side cross-sectional view of the plungerless aspiration and/or injection device of FIG. 2A, wherein the manner in which the device is used for aspiration is illustrated;

FIG. 2C is a side cross-sectional view of the needle and bulb of the plungerless aspiration and/or injection device of FIG. 2A, wherein the housing has been removed from the device to better illustrate the needle and bulb;

FIG. 2D is another side cross-sectional view of the needle and bulb of the plungerless aspiration and/or injection device of FIG. 2A, wherein the bulb is shown being compressed for active injection;

FIG. 2E is a side cross-sectional view of an alternative needle and bulb of the plungerless aspiration and/or injection device of FIG. 2A, where the bulb is provided with a drainage hole therein, and the bulb is shown being used for passive aspiration or drainage;

FIG. 2F is still another side cross-sectional view of the needle and bulb of the plungerless aspiration and/or injection device of FIG. 2A, wherein the bulb is shown being compressed for implantation of an implant;

FIG. 2G is yet another side cross-sectional view of the needle and bulb of the plungerless aspiration and/or injection device of FIG. 2A;

FIG. 2H is still another side cross-sectional view of the needle and bulb of the plungerless aspiration and/or injection device of FIG. 2A, wherein an implant is illustrated in the needle of the device;

FIG. 2I is a side cross-sectional view of a plungerless aspiration and/or injection device, according to yet another illustrative embodiment of the invention, wherein the bulb of the device is incrementally compressed using a flexible angulated step ladder-like structure;

FIG. 3A is a side cross-sectional view of an eye, according to an illustrative embodiment of the invention, wherein the needle tip of the plungerless aspiration and/or injection device has not yet penetrated the cornea of the eye;

FIG. 3B is another side cross-sectional view of the eye of FIG. 3A, wherein the needle tip of the plungerless aspiration and/or injection device has penetrated the cornea of the eye so that aqueous fluid is able to be aspirated from the anterior chamber of the eye;

FIG. 3C is yet another side cross-sectional view of an eye, wherein the needle tip of the plungerless aspiration and/or injection device has penetrated the side of the eye so that vitreous fluid is able to be aspirated from the vitreous cavity of the eye;

FIG. 3D is still another side cross-sectional view of an eye, wherein the plungerless aspiration and/or injection device is shown being used for injection or implantation in the anterior chamber of the eye, and the device is additionally shown being used for injection or implantation in the vitreous cavity of the eye;

FIG. 3E is yet another side cross-sectional view of an eye, wherein the plungerless aspiration and/or injection device is shown being used for injection or implantation in the sub-conjunctival space of the eye;

FIG. 3F is still another side cross-sectional view of an eye, wherein the plungerless aspiration and/or injection device is shown being used for injection or implantation in the suprachoroidal space of the eye;

FIG. 3G is yet another side cross-sectional view of an eye, wherein the plungerless aspiration and/or injection device is shown being used for injection or implantation in the subretinal space of the eye;

FIG. 3H is yet another side cross-sectional view of an eye, wherein the plungerless aspiration and/or injection device is shown being used for injection or implantation in the retrobulbar space of the eye;

FIG. 4 is a side cross-sectional view of a plungerless aspiration and/or injection device, according to another illustrative embodiment of the invention, wherein the needle of the device is in a retracted position, and the housing is provided with an inverted frustoconical end portion;

FIG. 5 is a partial side cross-sectional view of a distal end of the plungerless aspiration and/or injection device of FIG. 4, wherein the needle of the device is in a retracted position;

FIG. 6 is a partial side cross-sectional view of a distal end of the plungerless aspiration and/or injection device of FIG. 4, wherein the needle of the device is in an extended, exposed position;

FIG. 7 is a drawing of a longitudinal section of a human eye;

FIG. 8 is an enlarged diagrammatic illustration of the circled area 2 of FIG. 7 showing detailed retinal structures;

FIG. 9 shows the eye of FIG. 7 with a cannula delivering particles to the retina in accordance with one embodiment of the invention;

FIG. 10 is an enlarged diagrammatic illustration of the circled area 4 in FIG. 9 showing particles jetting from a cannula and dispersing throughout retinal structures;

FIGS. 11A-B is a schematic structure of an activated biodegradable silicone or luminescent quantum dot;

FIG. 12 schematically shows synthesis of a cell-penetrating peptide (CPP); and

FIG. 13 shows the chemical structure of an activated fluorescent dye.

Throughout the figures, the same elements are always denoted using the same reference characters so that, as a general rule, they will only be described once.

### DETAILED DESCRIPTION OF EMBODIMENTS OF THE INVENTION

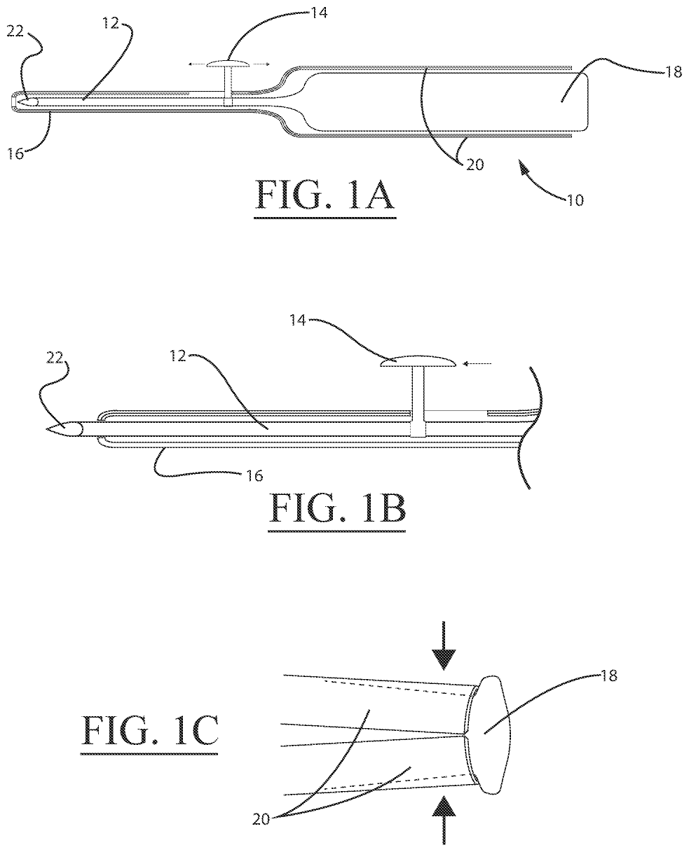

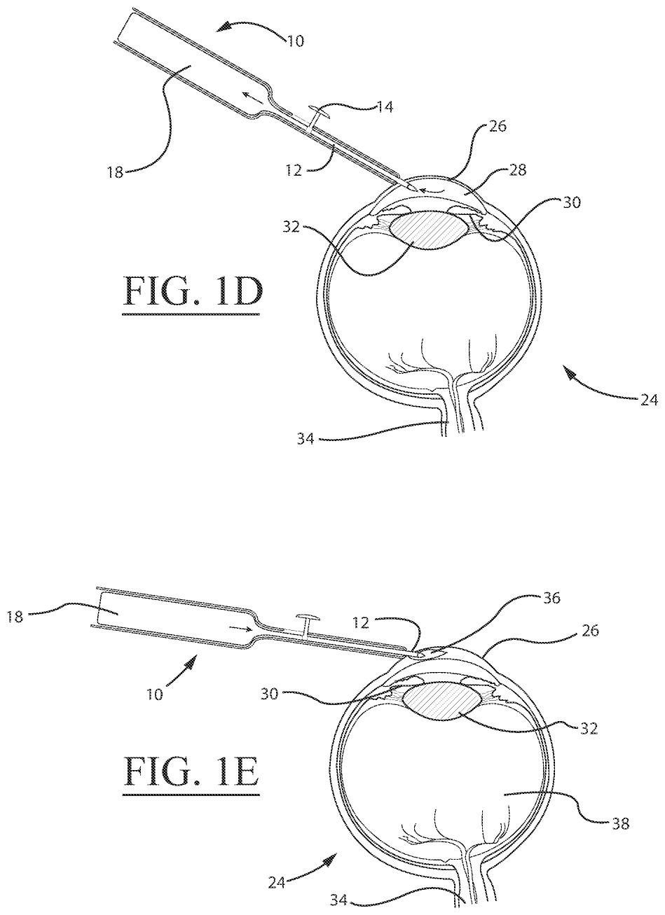

In one embodiment, there is a need for a simplified system that can be easily controlled by thumb and index fingers for injection of fluid or implantation of small sized implants, for example, drug delivery of polymeric nano- and micro-particulates or injecting a medication in solution or as emulsion or withdrawing a precise amount of fluid without moving the instrument that could damage the wall of the cavity, such as an artery or vein or the wall of the body cavity, such as the anterior chamber damaging the cornea, lens, or the iris of the eye and/or the vitreous cavity without damaging the retina, etc. (refer to FIGS. 1A-3H).

In one embodiment, the instrument 10 is composed of a housing where its front end covers a movable needle 12 and its back is like a flexible fork 20 or spatula-like extension covering an elongated semi-elastic compressible, silicone bulb reservoir 18 made of silicone or the like, rubber, or elastic polymer (see FIGS. 1A-1E).

In particular, as shown in the illustrative embodiment of FIGS. 1A-1C, the plungerless aspiration and/or injection device 10 comprises a housing 16; a needle portion 12 disposed in the housing 16, the needle portion 12 configured to be selectively retracted and extended by a user, the needle portion 12 comprising a needle tip 22 configured to be inserted into tissue of a patient for aspiration, injection, and/or implantation; and a bulb portion 18 disposed in the housing 16, the bulb portion 18 defining a fluid containing cavity that is fluidly coupled to the needle portion 12, and the bulb portion 18 being elastically deformable so that the user is able to perform the aspiration, the injection, and/or the implantation on the patient by manipulating the bulb portion 18.

Referring again to FIGS. 1A-1C, it can be seen that, in the illustrative embodiment, the plungerless aspiration and/or injection device 10 further comprising a knob 14 for manipulating the needle portion 12, the knob 14 being slidably disposed within a slot of the housing 16. When the knob 14 is moved in a first direction by the user, the needle portion 12 is retracted into the housing 16. Conversely, when the knob 14 is moved in a second, opposite direction by the user, the needle portion 12 is extended out of the housing 16. In the illustrative embodiment, the bulb portion 18 is connected to the needle portion 12 such that the bulb portion 18 is displaced with the needle portion 12 when the knob 14 is moved in the first direction or second direction by the user.

In the illustrative embodiment, as best shown in FIG. 1C, the housing 16 comprises elastically deformable forked blades 20 disposed over the bulb 18, the elastically deformable forked blades 20 configured to be depressed by the user between a thumb and index finger of the user so as to more controllably regulate a flow of fluid out of, or into the needle tip 22. The forked blades 20 may be spring-loaded to return to their undepressed state after being depressed by the user.

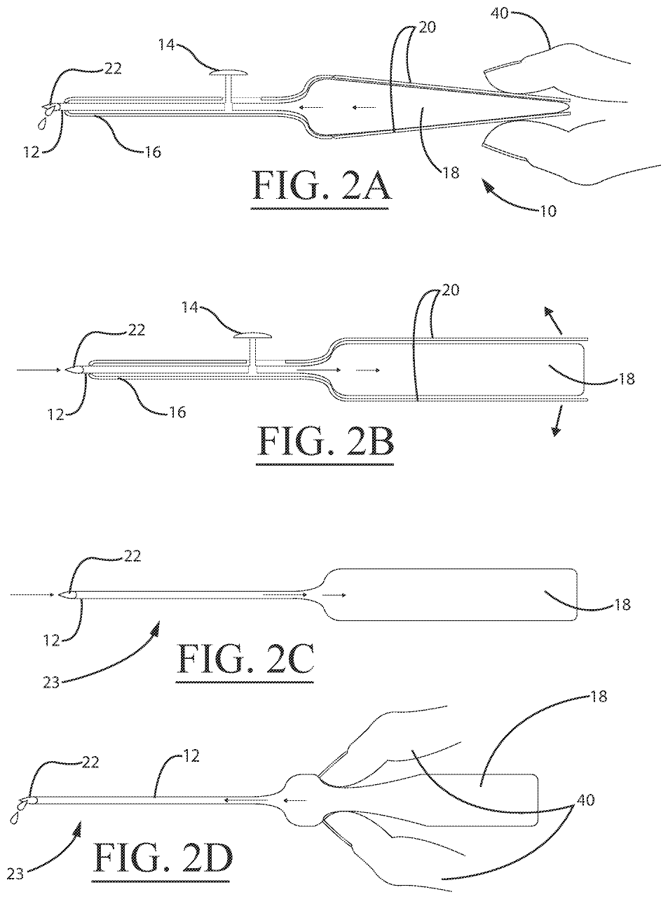

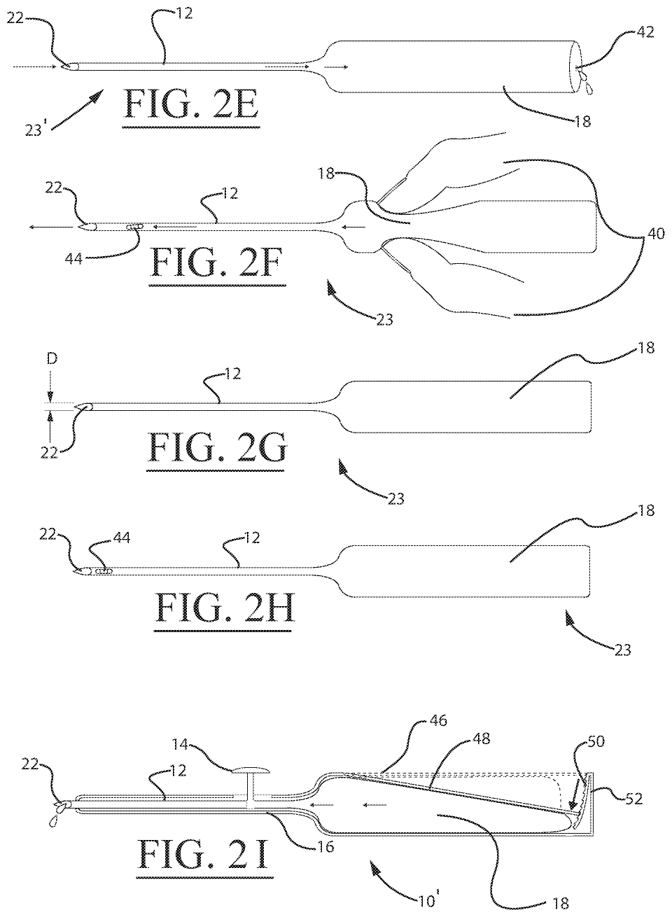

In FIG. 2A, the forked blades 20 of the illustrative plungerless aspiration and/or injection device 10 are shown being compressed by fingers of a user during injection of a fluid disposed in the bulb portion 18 of the device. In FIG. 2B, the forked blades 20 of the illustrative plungerless aspiration and/or injection device 10 are shown being displaced outwardly during aspiration.

The internal portion 23 of the illustrative plungerless aspiration and/or injection device 10 without the housing 16 is depicted in FIG. 2C. As shown in this figure, the internal portion 23 of the illustrative device 10 includes the needle portion 12 and the bulb portion 18. FIG. 2G depicts a view of the internal portion 23 of the illustrative device 10 similar to FIG. 2C, except that the needle diameter D of the needle portion 12 is labeled. In FIG. 2D, the bulb portion 18 of the internal portion 23 of the illustrative device 10 is shown being compressed by fingers 40 of a user during injection of a fluid. In FIG. 2E, an alternative embodiment of the internal portion 23′ of the illustrative device 10, which has a drainage hole 42 disposed in the bulb portion 18, is shown being used for passive aspiration or drainage. In FIG. 2F, the bulb portion 18 of the internal portion 23 of the illustrative device 10 is shown being compressed by fingers 40 of a user during the implantation of an implant 44. FIG. 2H depicts a view of the internal portion 23 similar to FIG. 2F, except that the implant 44 is illustrated in the needle portion 12 prior to being injected when the bulb portion 18 is undeformed.

In FIG. 1D, an exemplary manner in which an aqueous fluid may be aspirated from the anterior chamber 28 behind the cornea 26 of an eye 24 using the device 10 is illustrated. The eye 24, as illustrated in FIG. 1D, further includes an iris 30, a lens 32, and an optic nerve 34. In FIG. 1E, an exemplary manner in which a substance may be injected into an intracorneal cavity 36 of an eye 24 using the device 10 is illustrated. The eye 24, as illustrated in FIG. 1E, further includes an iris 30, a lens 32, an optic nerve 34, and a vitreous cavity 38.

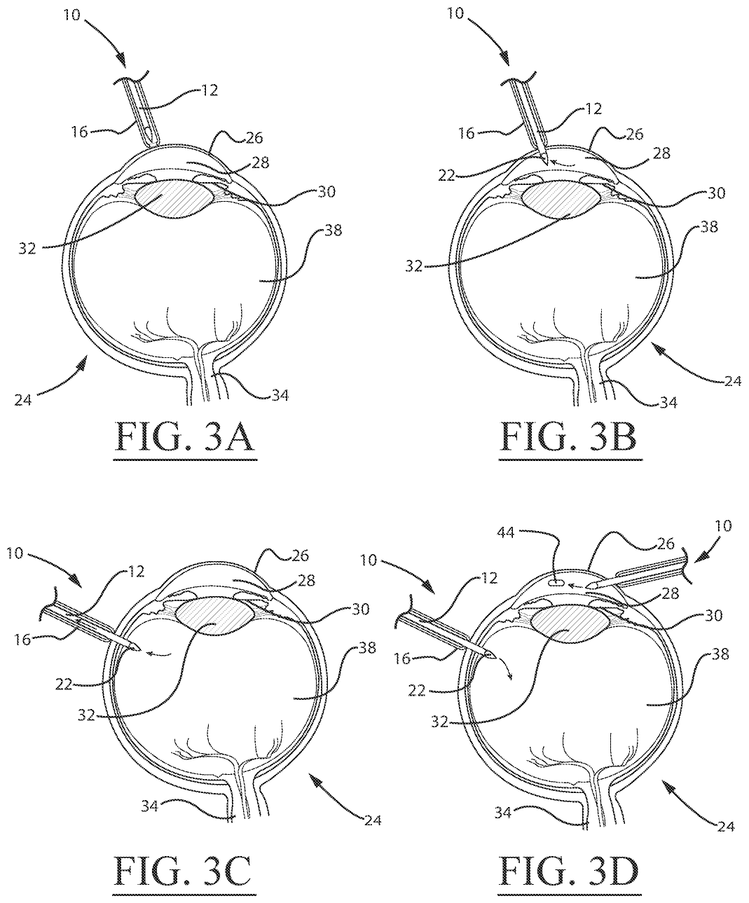

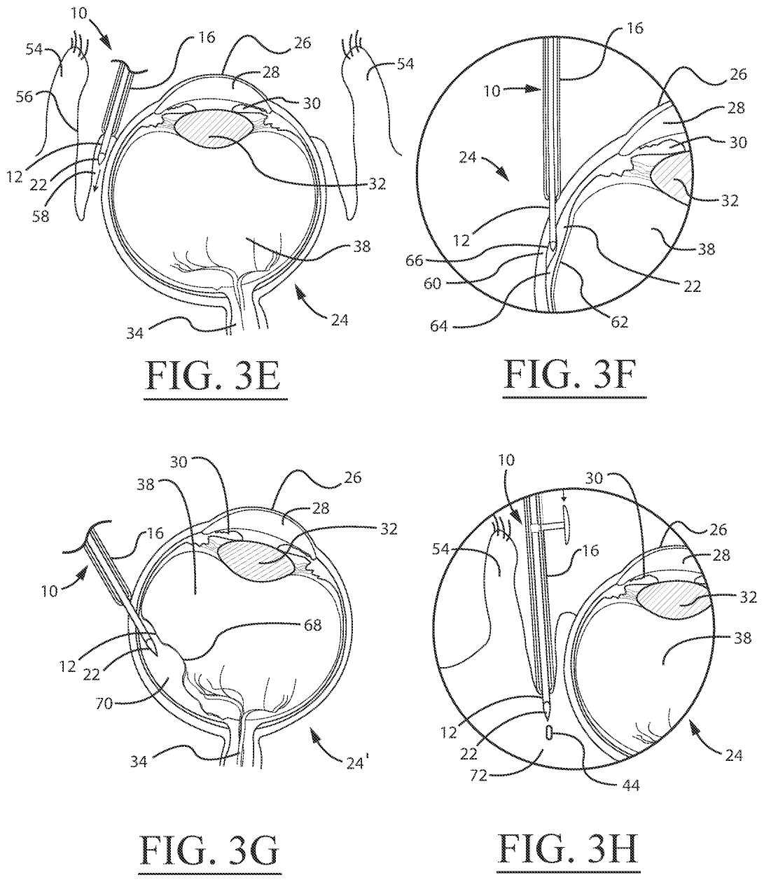

In FIGS. 3A and 3B, another exemplary manner in which an aqueous fluid may be aspirated from the anterior chamber 28 behind the cornea 26 of an eye 24 is illustrated. More specifically, FIG. 3A depicts the tip of the plungerless aspiration and/or injection device 10 proximate to the surface of the cornea 26 while the needle portion 12 of the device 10 is still retracted in the housing 16 of the device 10. FIG. 3B depicts the needle portion 12 of the device 10 inserted into the cornea 26, and aqueous fluid being aspirated from the anterior chamber 28 of the eye 24. The eye 24, as illustrated in FIGS. 3A and 3B, further includes an iris 30, a lens 32, an optic nerve 34, and a vitreous cavity 38.

In FIG. 3C, an exemplary manner in which vitreous humour may be aspirated from the vitreous cavity 38 of an eye 24 using the device 10 is illustrated. In FIG. 3D, an exemplary manner in which a fluid or implant may be injected into the vitreous cavity 38 of an eye 24 using the device 10 is illustrated. Also, in FIG. 3D, another device 10 is shown being used to inject a fluid or implant 44 in the anterior chamber 28 of the eye 24. The eye 24, as illustrated in FIGS. 3C and 3D, further includes a cornea 26, an iris 30, a lens 32, an optic nerve 34, and a vitreous cavity 38.

In FIG. 3E, an exemplary manner in which a fluidic substance or implant may be injected into the subconjunctival space 58 of an eye 24 using the device 10 is illustrated. The eye 24, as illustrated in FIG. 3E, includes a cornea 26, an anterior chamber 28, iris 30, a lens 32, an optic nerve 34, a vitreous cavity 38, lids 54, and conjunctiva 56.

In FIG. 3F, an exemplary manner in which a fluidic substance or implant may be injected into the suprachoroidal space 66 of an eye 24 using the device 10 is illustrated. The device 10 could also be used in a similar manner for aspiration from the suprachoroidal space 66. The eye 24, as illustrated in FIG. 3F, includes a cornea 26, an anterior chamber 28, iris 30, a lens 32, a vitreous cavity 38, a sclera 60, retina 62, and choroid 64.

In FIG. 3G, the eye 24′ with cornea 26, anterior chamber 28, iris 30, a lens 32, optic nerve 34, and vitreous cavity 38 has a detached retina 68 that produces a subretinal space 70. The plungerless aspiration and/or injection device 10 may be used to aspirate a fluid from the subretinal space 70 during a medical procedure performed to reattach the retina 68.

In FIG. 3H, an exemplary manner in which a fluidic substance or implant 44 may be injected into the retrobulbar space 72 of an eye 24 using the device 10 is illustrated. The eye 24, as illustrated in FIG. 3H, includes a cornea 26, anterior chamber 28, iris 30, a lens 32, a vitreous cavity 38, and lid 54.

In one embodiment, the injection is done by actively pressing gently over the flexible spatula and the bulb inside it (refer to FIGS. 2A-2H).

In one embodiment, the needle is pushed in the tissue with the index finger moving the knob located in a slot in the external housing and while the needle is inside the cavity, by releasing slowly the bulb's semi-elastic body expanding automatically and aspirating gradually a small volume of the liquid from the fluid containing cavity, so that it can subsequently be analyzed outside the body using known diagnostic tools such spectrometer, microscope, staining for recognition of various bacteria, fungi, parasites, or viruses, or mass-spectroscopy, PCR, Raman spectroscopy or surface-enhanced Raman spectroscopy technique, enzyme-linked immunosorbent assay (ELISA) test strips, dot blots, solutions on slides, etc. In one embodiment, the biological fluid is anterior chamber fluid and the detection reagent detects a cytokine, etc.

In one embodiment, under normal conditions, the needle tip is retracted and inside the outer metallic, plastic, or glass tube housing, etc. This permits positioning of the instrument with its dull tip end of outer housing to be moved over the tissue surface or inside the tissue and to select a desired location without scratching or cutting the sensitive tissue surface, such as the cornea, skin, mucosa, or choroid, etc.

In one embodiment, the injector carries functionalized antibody-coated nanoparticles with pluralities of nanoparticles conjugated with checkpoint inhibitors and oncolytic viruses to be injected inside a tumor to release the medication as a response to external energy, such as laser, alternating magnetic field, or a focused ultrasound, to treat an external or internal tumor, such as melanomas or precancerous melanoma inside the eye or conjunctiva, lid tumors, breast cancer, prostate cancer, skin tumors, such as squamous cancer or basal cell cancer of the skin.

In one embodiment, the needle can be moved forward or backward by moving a knob connected to the inner needle forward or backward, since the silicone bulb is connected to the needle's end, but is elastic and not fixed to its housing, it can also travel inside the housing forward or backward by the same exposed knob (see FIGS. 1A-1E and FIGS. 2A-2B).

In one embodiment, the needle can be any size with the lengths of 1 mm to 100 mm or more.

In one embodiment, the needle has a diameter of 0.001 mm to 1 mm or more.

In one embodiment, the bulb reservoir by its connection to outside is filled with the air, gas, etc.

In one embodiment, the bulb is filled with a liquid solution or emulsion.

In one embodiment, the bulb is filled with an emulsion containing nanoparticles, microparticles, or larger particles.

In one embodiment, the bulb reservoir contains a viscoelastic material.

In another embodiment, the viscoelastic contains medication or a photosensitizer, such as riboflavin or Porphyrin derivatives, methylene blue or indium, platinum, rhodium plus albumin, eosin, rose Bengal, phthalocyanines, carotenoids, semiconductor nanoparticles, etc. that is activated under ultraviolet (UV) or another radiation to crosslink proteins of an organism.

In one embodiment, the injector is filled with a photosensitizer and an antibiotic, an antifungal, an antiviral, an anticancer medication, anti-parasites, etc.

In one embodiment, the injector is filled with functionalized thermosensitive nanoparticles, such as gold, iron oxide, carbon nanotubes, or thermosensitive polymers, such as liposomes, solid lipids, micelles, nanobubbles, etc. to carry medications in the form of slow release compounds and release the medication after administration.

In one embodiment, the injector carries functionalized antibody-coated nanoparticles with pluralities of nanoparticles conjugated with checkpoint inhibitors and oncolytic viruses to be injected inside a tumor to release the medication as a response to external energy, such as a laser, alternating magnetic field, or a focused ultrasound, to treat an external or internal tumor, such as melanomas or precancerous melanoma, inside the eye or conjunctival or lid tumors, breast cancer, prostate cancer, skin tumors such as squamous cancer or basal cell cancer of the skin.

In one embodiment, the fluid is a viscoelastic material and contains a medication in form of an emulsion containing nanoparticles or micro-particles having a diameter of 1-1000 nm.

In one embodiment, the fluid is a viscoelastic material (e.g., hyaluronic acid or 0.01-1% or more).

In one embodiment, the viscoelastic material is made from another compound.

In another embodiment, the viscoelastic material contains a medication in the form of polymeric slow release nanoparticles or micro-particles of 1-1000 microns or more.

In one embodiment, the polymeric slow release nanoparticles or micro-particles or the implant injected to release medication is made from liposomes, micelles, nano- and micro-particulates of polylactic, polycyclic acid, or in combination with PGLA or porous silicone, nanoparticles of hydrogel, or polyesters, such as polycaprolactone, or other compounds such as chitosan, solid lipids, collagen, alginate dendrimers, metallic nanoparticles such as gold tubes filled with polymeric medications, etc.

In one embodiment, the nanoparticles can be functionalized to attach to certain cells, organisms, bacteria, etc.

In one embodiment, the viscoelastic material carries with it an implant with a diameter of 0.02-2 mm or more.

In one embodiment, the implant can have a length of 0.01-5 mm or more.

In one embodiment, the inner tube is filled with a polymeric drug delivery implant with a diameter of 20-1000 microns and a length of 0.02-100 mm or more as needed.

In one embodiment, the drug or fluid administration, etc. does not require an incision to be made in the tissue (e.g., with a knife, etc.), but the drug or the fluid is administered with the needle's sharp end, which does not require an incision for injection or a suture for its closure.

In one embodiment, the injection is done preferably in an angulated direction to the surface of the tissue that permits the tissue around the injection to close like a valve by the tissue pressure or increased pressure inside the tissue or the cavity after the injection preventing expulsion of the medication or the implant.

In one embodiment, the housing of the needle is positioned over the desired area, then the sharp needle's tip is slowly or gradually moved forward inside the tissue under observation to see the needle tip (e.g., inside the corneal pocket or inside the anterior chamber, etc.) by moving the instrument's knob forward to the desired distance and a desired direction in the tissue of the cavity, then compressing the medication-filled elastic bulb reservoir to release the medication under the observation of the needle's tip in the desired place (refer to FIGS. 2A-2I and FIGS. 3A-3H).

In one embodiment, the housing of the needle is positioned over the desired area, then the sharp needle tip is slowly or gradually moved forward inside the tissue under observation to see the needle tip by moving the instrument's knob forward to the desired distance and desired direction in the tissue or inside the cavity, then compressing the elastic bulb through the elastic forked housing over the bulb. The elastic thin-walled fork over the bulb provides a more controllable pressure to the bulb filled with the medication, etc. to be compressed between the thumb and index finger to slowly release the medication in the desired area or stop at a desired time (see FIGS. 2A, 2B, 2D, and 2F).

In one embodiment, the housing of the needle is positioned over the desired area, then the sharp needle tip is moved slowly or gradually forward inside the tissue under observation to see the needle tip by moving the instrument the knob forward to a desired distance and desired direction in the tissue of the cavity while the bulb is compressed, then the compression of the bulb is released slowly to aspirate fluidic biopsy to a desired volume, the biopsy fluid for subsequent analysis as described (refer to FIGS. 2C and 2E). In one embodiment, the sharp tip of the needle is used inside the tissue, e.g., inside the cornea after femtosecond laser application or a Small Incision Lenticule Extraction (SMILE) procedure to dissect the bridges of the tissue with ease.

In one embodiment, the instrument is equipped with a graded flexible guard that indicates how much fluid or medication is injected in a cavity (e.g., vitreous cavity, in the anterior chamber, under the conjunctival, inside the vitreous cavity, in the suprachoroidal space, under the retina, or anywhere else—see FIG. 2I and FIGS. 3A-3H).

In one alternative embodiment, as shown in FIG. 2I, by pressing the flexible guard 46 over the bulb 18, a fluid is injected using the plungerless aspiration and/or injection device 10′. In this alternative embodiment, the housing 16 is also equipped with a flexible step ladder stopper 50 either as a rectangular plate or a ring-shaped plate with deep grooves, by pressing one of the forked blades 48 of the housing 16, it falls inside first groove indicating, e.g., 0.05 ml of the fluid is expelled out while making a clicking sound. In one embodiment, one can build the stopper so that each step (groove) would indicate how much more fluid volume is injected inside a cavity from 0.05 to 0.5 ml to 0.1 ml-2.05 ml, etc. as needed precisely (refer to FIG. 2I).

As shown in the alternative embodiment of FIG. 2I, to inject a predetermined amount of a fluid, a user presses the flexible guard 46 over the bulb 18 until the proximal end of the forked blade or pivotable lever 48 seats within one of the grooves in the angulated stepped internal wall 50 of the plungerless aspiration and/or injection device 10′, wherein the groove corresponds to a predetermined amount of fluid being discharged from the device 10′. In FIG. 2I, it can be seen that the proximal end of the device 10′ is closed by an end cap 52, which is attached to the angulated stepped internal wall 50.

In one embodiment, the needle is inserted inside the tissue or a cavity and the medication, fluid, gas or implant is expelled out by simply applying pressure to the bulb.

In one embodiment, the instrument can be used as a passive system for collection of fluid biopsy from the blood or a body cavity by creating a small hole in the wall of the bulb to release fluid from the body cavity through the needle to the silicone bulb to outside where it can be collected as desired (refer to FIG. 2E).

In one embodiment, a silicone tube is connected to the needle and the external end is connected to an elastic silicone tube to drain the fluid out of the eye cavity in a passive way that can be clamped at any time as known in the art.

In one embodiment, the device permits removal of a small amount of fluid of 0.1 microliters to milliliter, etc. as needed for the analysis either in bacterial, fungal, viruses, parasites, etc. infection, or malaria detection, circulating tumor cells, exosomes, micro-RNS, micro-DNA, antibodies indicating one or other disease process in the eye fluid or elsewhere in a body cavity.

In one embodiment, the needle is inserted in the suprachoroidal space while a thin flexible metallic wire is pushed through it to drain fluid of a pigment epithelial detachment, by inserting the wire from the back into a pigment epithelial detachment space and draining passively the fluid in the choroidal space in the pigment epithelial detachment (see FIG. 3F).

In one embodiment, the silicone bulb can be injected with the medication in liquid or emulsion form or an implant form that can pass through the wall diameter of the needle prior to release of the medication or implant in the eye cavity, corneal cavity, subconjunctival space in the lens capsule after cataract extraction in the lens capsule, or in the sub-retinal space or in the choroid.

In one embodiment, the instrument's needle can be inserted in any place in the eye (refer to FIGS. 3A-3H) or outside the eye (e.g., in the joints, mouth, bladder, vagina, nose, ear, etc., cerebrospinal fluid, etc. to take either a fluid biopsy or injected medication).

In one embodiment, the flexible bulb is used to drop the medication as a drop, or spray the liquid medication of emulsion over or inside a body cavity or over the cornea or under the lid or over the conjunctiva, or over a body's mucosa by compressing rapidly the flexible tail of the instrument with the bulb reservoir to spread the medication in the cavity.

In one embodiment, the medications that are administered using this instrument are steroids, anti-inflammatory agents, NSAIDs, Rock inhibitors, integrin inhibitors, GSK inhibitors alone or in combination in slow release nanoparticle polymers, such as polylactic or glycolic acid, micelles, liposomes, porous silicon, or polyester, and is administered precisely to release the medication in the cornea or inside a body tissue or cavity for a long time after implantation.

In one embodiment, the medications that are administered using this instrument are anti-inflammatory agents, such as Rock inhibitors, GSK inhibitors, integrin inhibitors, alone or in combination with immunosuppressants such as cyclosporine, Mycophenolic acid, ascomycin, metalloproteinase inhibitors, such as doxycycline, tetracycline, etc. or low molecular weight heparin, alone or in combination in slow release nanoparticle or microparticle polymers, such as polylactic or glycolic acid, micelles, liposomes, porous silicon, or polyester to release the medication in the cornea or inside a body tissue or cavity for a long time after implantation.

In one embodiment, the unit can be prefilled with specific non-toxic doses of antibiotics, antivirals, anti-parasites, anti-fungals, or antivirals, etc. in a solution or emulsion of nanoparticles for slow release of the medication.

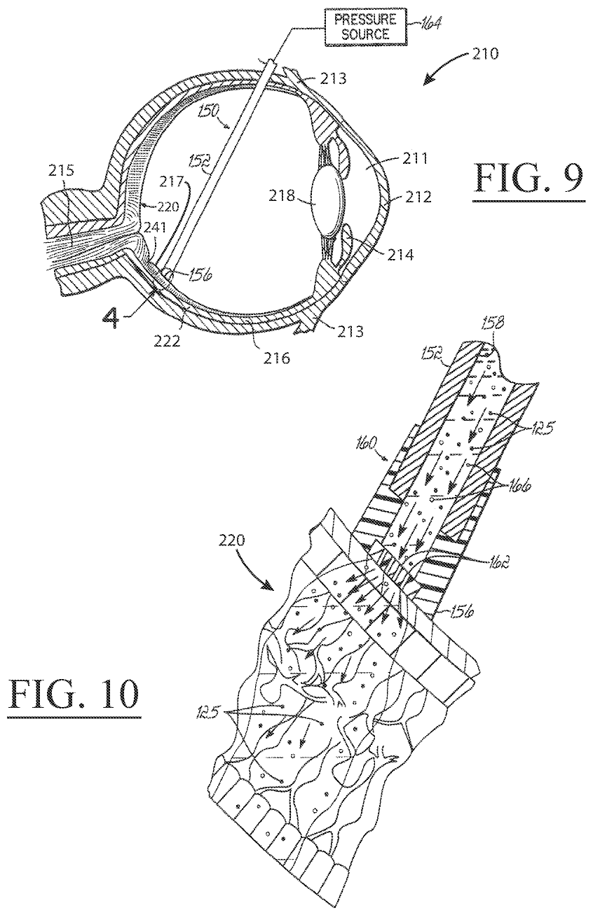

In one embodiment, the disease process involves the cornea, glaucoma, dry eye, uveitis, retinal detachment, refractive surgery and corneal implantation, optic nerve inflammation, retinitis pigmentosa, diabetic retinopathy, age-related macular degeneration wet or dry form, glaucoma, scleritis and the medication is injected in the cavity or inside the tissue as shown in FIG. 9.

In one embodiment, the instrument can be made sterile with pre-filled medication as a disposable unit to deliver the medication to the quantity/volume as needed.

In one embodiment, the unit can have one or multiple medications, packaged sterile with pre-filled medications as a disposable unit for single use on a patient.

In another embodiment, the unit is sterilized and packaged, and is used for aspiration of a liquid biopsy as a disposable unit for single use on a patient.

In one embodiment, the plungerless injector and aspirator is used for vaccination.

In one embodiment, vaccination induces an immune response to an antigen where the antigen can be attenuated or dead viruses, attenuated or dead bacteria, attenuated or dead fungi, attenuated or dead parasites, or attenuated or dead tumor cells.

In one embodiment, the instrument is used for a single injection, and then disposed of without being used again.

In one embodiment, the vaccine can be administered intramuscularly, subcutaneously, intravenously, intra-arterially, or inside a body's cavity as a drop or spray in a liquid or aerosolized form through the nose, mouth, vagina, bladder, skin or injected in a tumor, inside a lesion or can be taken orally sublingually as emulsion of nanoparticles or microparticles, or through the intestinal tract, etc.

In one embodiment, the needle part of the injector can have a diameter of 10 microns to 2 mm or more with a length of 0.1 mm to 50 mm or more.

In one embodiment, the needle is covered with anticoagulant so that the blood does not coagulate inside the needle for venous micro-puncture or micro-aspiration to obtain a small amount of fluid for diagnostic purposes.

In one embodiment, the antiviral vaccine is made from dead viruses, or a part of the virus such as a lipid, protein, or polysaccharide, etc., or a part of the fungi, bacteria, parasites, or tumor cells, or it is made from mRNA or DNA or a part of them, or neutralized or crosslinked RNA or DNA of the viruses, bacteria, fungi, parasites, or tumor cells.

In one embodiment, the antigenic material of the vaccine is combined with either one or two or more medications, such as antivirals, antibacterials, anti-parasites, or anti-tumor medications, or a combination thereof.

In one embodiment, the instrument's chamber is filled with a liquid, semisolid, air or a gel or a mixture of them that can be injected or can create a spray for internal or external tissue, intranasally, inside the mouth, or inside a body cavity, such as vagina, uterus, bladder, or rectum.

In one embodiment, the instrument is used to withdraw blood from a vein or artery using the smallest possible needle since the blood will flow as soon as the needle tip enters the vessels, with the wall of the needle being coated with heparin to prevent blood clot formation.

In one embodiment, the outer tube of the needle can be 0.1 mm in length to 2.0 cm or more in length, and the active part of the unit can be of the same length or larger depending on where it is inserted at a desired distance outside or inside the body cavity.

In one embodiment, the cavity can be created inside a tissue with the sharp edge of the needle so that the rest of the needle and its outer tube can be placed inside the cavity and its flexible balloon can be filled with a medication, a solid implant, a semisolid implant, or an emulsion of nanoparticles or microparticles or larger particles, such as solid or semisolid particles of a few millimeters, or can be injected along with a fluid or viscoelastic material to lubricate the way of the injection (e.g., one creates a subcutaneous cavity, intracorneal cavity, a sub-conjunctival cavity, sub-mucosal cavity). The instrument is pushed in the cavity or inside an existing cavity such as the nose, mouth, etc. to spray the nose, mouth, pharynx, or inside a body cavity, such as in the bladder, entering through the ureter, etc.

In one embodiment, the plungerless injector and/or aspirator is in a form of a catheter with a double tube, nasal aspiration, and drop or spray. In this embodiment, the nozzle of the plungerless injector and/or aspirator is made of a solid injectable needle and its outer tube is made out of a soft synthetic polymer with various lengths that can be used to carry medication(s) through the ureter into the bladder or to empty the bladder.

In one embodiment, the inner tube can carry anti-neoplastic medications along with cellular inflammatory pathway inhibitors, antibacterials, antivirals, antifungals, anti-tumoral medication, etc. that can be applied to the wall of the bladder or ureter for irrigation and aspiration, etc.

In one embodiment, the tip of the aspiration-injection needle is made from solid or semisolid matter so that the sharp tip of the needle penetrates the skin or mucosa, or the corneal tissue for 0 to 200 microns or more where the compressible section of the unit is filled with non-toxic dark nanoparticles or microparticles are injected inside the desired part of the central cornea creating a ring of 1-3 mm tattooed area leaving a central clear portion of 1-2 mm transparent area for the light to pass through it functioning as a pinhole to treat presbyopia, myopia, and reduce the astigmatic aberrations of the cornea.

In one embodiment, a central 2-3 mm diameter portion of a corneal inlay is tattooed to create a dark spot and after tattooing the central 1.6 mm circle is removed with a trephine to create a pinhole before or after its implantation as a corneal inlay.

In one embodiment, the plungerless injector and/or aspirator is used for the machine-assisted injection of the skin, mucosa for a desired superficial or a deeper layer of the skin or sub-cutaneous tissue where the needle deposits desired medication to a lesion, or the polymeric nanoparticles act as a slow release implant for 3-6 months or more.

In one embodiment for vaccination or injection inside the tissue, the conical or frustoconical-shaped head of the unit is pressed over the skin to numb the subcutaneous nerves prior to the injection, thus creating a painless administration of a vaccine or injection inside the knee joints or the shoulder area, spinal cord, or injection inside the finger joints or toe joints, where the medication may be included with or without a viscoelastic that can be injected for cosmetic reconstruction of the face using Botox, etc.

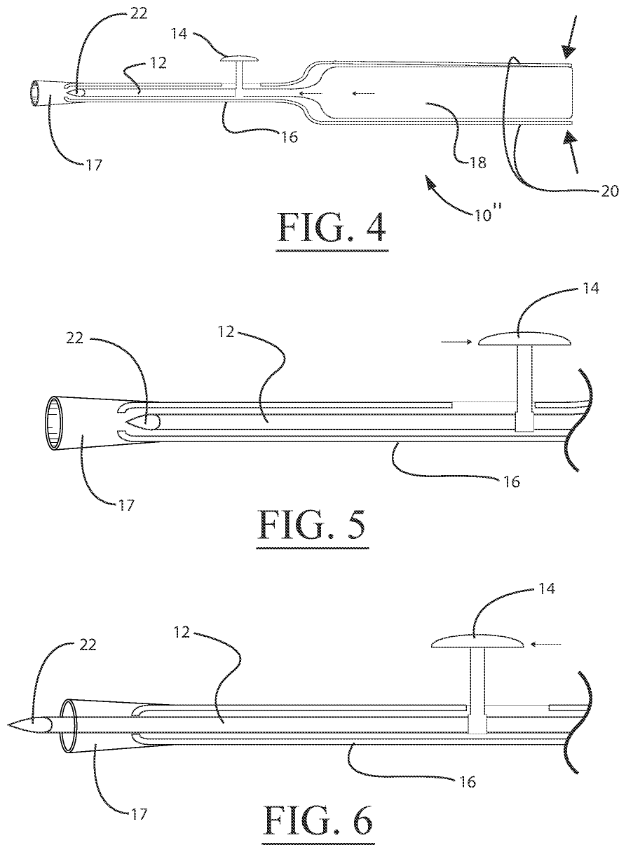

Another illustrative embodiment of the plungerless aspiration and/or injection device is shown in FIGS. 4-6. The plungerless aspiration and/or injection device 10″ depicted in FIGS. 4-6 includes a frustoconical-shaped head or end portion (i.e., an inverted frustoconical-shaped head or end portion). In the illustrative embodiment of FIGS. 4-6, the plungerless aspiration and/or injection device 10″ comprises a housing 16, the housing 16 including an inverted frustoconical end portion 17 extending outwardly from the housing 16, the inverted frustoconical end portion 17 having an outer edge that is configured to be pressed against the skin of a patient; a needle portion 12 disposed in the housing 16, the needle portion 12 configured to be selectively retracted and extended by a user, the needle portion 12 comprising a needle tip 22 configured to be inserted into tissue of the patient for aspiration, injection, and/or implantation, and the needle tip 22 configured to be extended beyond the outer edge of the inverted frustoconical end portion 17 of the housing 16 (as shown in FIG. 6); and a bulb portion 18 disposed in the housing 16, the bulb portion 18 defining a fluid containing cavity that is fluidly coupled to the needle portion 12, and the bulb portion 18 being elastically deformable so that the user is able to perform the aspiration, the injection, and/or the implantation on the patient by manipulating the bulb portion 18. In the illustrative embodiment, the outer edge of the inverted frustoconical end portion 17 of the housing 16 is configured to be pressed against the skin of the patient prior to insertion of the needle tip 22 of the needle portion 12 into the skin of the patient so as to reduce a pain sensation of the patient resulting from the insertion of the needle tip 22 of the needle portion 12.

Referring again to FIGS. 4-6, it can be seen that, in the illustrative embodiment, the plungerless aspiration and/or injection device 10″ further comprising a knob 14 for manipulating the needle portion 12, the knob 14 being slidably disposed within a slot of the housing 16. When the knob 14 is moved in a first direction by the user, the needle portion 12 is retracted into the housing 16. Conversely, when the knob 14 is moved in a second, opposite direction by the user, the needle portion 12 is extended out of the housing 16. In the illustrative embodiment, the bulb portion 18 is connected to the needle portion 12 such that the bulb portion 18 is displaced with the needle portion 12 when the knob 14 is moved in the first direction or second direction by the user.

In the illustrative embodiment, as best shown in FIG. 4, the housing 16 comprises elastically deformable forked blades 20 disposed over the bulb 18, the elastically deformable forked blades 20 configured to be depressed by the user between a thumb and index finger of the user so as to more controllably regulate a flow of fluid out of, or into the needle tip 22. The forked blades 20 may be spring-loaded to return to their undepressed state after being depressed by the user.

In one embodiment, the plungerless unit also can be used for administering medication in a slow release form to the scalp to bring nutrition or medication to the hair follicles, such as finasteride, etc. or Rock inhibitors for hair growth or along with hair transplantation to prevent inflammation or rejection of a transplant or to treat an infection, etc., or with the medication along with Rock inhibitors, Wnt inhibitors, GSK inhibitors, or integrin inhibitors to enhance cell proliferation or administering medications such as antibacterials, antivirals, anti-fungals, anti-neoplastic medications, etc.

In one embodiment, progenitor cells can be injected along with a Rock inhibitor, a Wnt inhibitor, or integrin inhibitors to encourage cell growth, such as corneal endothelial cell growth, or injected inside the retina or subretinal with nerve cell progenitors or mesenchymal stem cells, etc. to grow inside the tissue, e.g., in age-related macular degeneration, etc.

In one embodiment, the system can be placed inside the joints, inside a cutaneous lesion, or inside the anterior chamber of the eye, in the nose, mouth, etc. to take fluid samples, etc. for biopsy, chemical analysis, or to recognize an infection or a malignancy, etc.

In one embodiment, the unit is used to spray or nebulize medications, or a vaccine, etc., and can be administered intranasally or for inhalation through the nose or mouth or spraying medication on the surface of the skin or mucosa such as the mouth, nose, vagina, or anal application of various medications to assist in treating patient with severe constipation or deliver microbiomes, or to treat an infection, such as viral, bacterial, etc. so that the medication is absorbed through the nasal mucosa via the olfactory nerves or the 5th nerve trigeminus, transporting medication to the brain to treat a brain infection or treat a patient with severe depression (e.g., applying ketamine) or Rock inhibitors for a patient with traumatic brain injury, Alzheimer's disease or Parkinson's disease, or administered to the eye for treatment of glaucoma with the medication as a nasal spray, conjunctival spray, subconjunctival injection of polymeric slow release nanoparticles or microparticles alone or Rock inhibitors with mycophenolic acid or low molecular heparin as micelles or liposomes to prevent redness of the conjunctiva after topical administration of Rock inhibitors.

In one embodiment, the unit delivers encapsulated medications, such as medications in micelles or liposomes that can break after a period of time to release appropriate medications.

In one embodiment, a vaccine administered superficially or subcutaneously by injection followed with or without application of focused ultrasound to enhance the spread of the vaccine or as a therapeutic vaccine with anti-neoplastic medications and Rock inhibitors as an adjuvant, antibacterial, antifungal, or antineoplastic medication with or without a monoclonal antibody or adjuvants, etc. to enhance the immune response in the body and locally kill the neoplasms, bacteria, viruses, fungi, or parasites.

In one embodiment, the unit is made disposable for a single use with a predetermined amount of medication to inject medications in a liquid, semi-liquid, gel, or as polymeric slow release nanoparticles or microparticles, etc. for slow release from the polymeric nanoparticles or microparticles injected at any desired place in the body.

In one embodiment, the flexible balloon is filled with a viscoelastic material and a semi-solid polymeric compound, such as a flexible intraocular lens that can pressed through the nozzle of the unit to be injected, e.g., after removal of a cataract inside the lens capsule just by compressing the balloon with two fingers or a glaucoma shunt can be injected in the Schlemm's canal through the trabecular meshwork of the eye.

In one embodiment, an intracorneal inlay is placed inside the balloon of the plungerless injector and/or aspirator unit filled with a viscoelastic fluid, where the corneal inlay is to be placed inside a corneal cavity prepared with a femtosecond laser and the conical or frustoconical-shaped tip of the unit cannot only penetrate the cornea, but also, if needed, create a cavity with its sharp needle and eject the corneal inlay into the corneal cavity to modify refractive error of the eye or enhance the mechanical properties of a soft cornea, such as in keratoconus.

In one embodiment, the unit is used for intracorneal injection or subconjunctival injection, suprachoroidal injection, or subretinal or intraocular injection, or in the lens capsule after cataract surgery delivering various medications, e.g., such as micelles, liposomes, gel, or as slow release polymeric nanoparticles or microparticles carrying the medications.

In one embodiment, piezoelectric nanoparticles or microparticles are injected with the use of the injector in the vitreous cavity to migrate in the retina, or injected under the retina to migrate under the retina gradually, or in the retina through the intercellular spaces of the retinal cells to stimulate the retinal cells when stimulated with the ultrasound from outside the eye.

In one embodiment, because the nanoparticles or microparticles have a small size, the particles can mold to the back side of the retina and the retinal pigment epithelium, while permitting nutrition and fluid to pass around the nanoparticles or microparticles to reach the entire thickness of the retina in contrast to the previously implanted plate of electrodes, etc. that would block nutrition of the retina (see e.g., Peyman et al., “Subretinal semiconductor microphotodiode array”, Ophthalmic Surgery and Lasers; Thorofare Vol. 29, Iss. 3, (March 1998): pp. 234-241, the disclosure of which is hereby incorporated by reference as if set forth in its entirety herein).

In one embodiment using an injector and aspirator carrying the nanoparticles or piezoelectric nanoparticles, a gene-CRISPR/cas9 complex can be administered through the vitreous cavity or preferably in the superior subretinal space so that with time the nanoparticles and microparticles migrate toward the sub-foveal space under the retina or through the intracellular spaces to reach the central retina and can be stimulated with ultrasound or light to enhance gene delivery after light or ultrasound stimulation, inducing an electrical cell membrane polarization or depolarization which opens the cell membrane pores permitting the genes to enter the cells cytoplasm in the macular area of the retina and target the mutated DNA, cut to remove it, or replace it with an unmutated DNA segment.

In one embodiment, the unit is used to deliver the medication as slow release polymers or gel in the vitreous cavity, subretinal space, retrobulbar injection, or for subcutaneous slow release of medications, or for nasal single dose inhalation, spray or nebulization for treatment of various diseases, such as allergies, bacteria, viruses, or fungi, etc., affecting the nose, eye, throat, bronchi, lung, or brain, etc.

In one embodiment, the piezoelectric nanoparticles or microparticles can be flexible piezoelectric microparticles, perovskite/wurtzite piezoelectric or Zno materials, or can be made biocompatible by encapsulating or coating them or using coupling agents such as phosphate, carbolate to modify the surface of the nanoparticles microparticles, or covering them with poly-L-Lysine, chitosan, etc. (e.g., refer to U.S. Pat. No. 10,022,457, entitled Methods to Regulate Polarization And Enhance Function of Cells, the disclosure of which is hereby incorporated by reference as if set forth in its entirety herein).

In one embodiment, the plungerless injector and/or aspirator is used either with a desired medication, e.g., an anti-VEGF or Rock inhibitor as topical drops on the corneal or conjunctiva or subconjunctival space, or along with slow release polymers in the vitreous, anterior chamber, or suprachoroidal space, or in the subretinal space to release medication slowly to penetrate retina for slow release of mediations or in conjunction with nerve progenitor stem cells or mesenchymal cells to migrate under the retina to the foveal area though the intercellular space or subretinal space gradually to prevent the foveal cell damage caused, e.g., by age-related macular degeneration dry or wet form, etc.

In one embodiment, the plungerless injector and/or aspirator is used either with a desired medication with neuronal progenitor cells, or along with slow release polymers in the suprachoroidal space, or in the subretinal space to release a medication, such as Rock inhibitors, Wnt inhibitors, GSK inhibitors, or integrin inhibitors to slowly penetrate the retina, or placed in the subretinal space for slow release of mediations, or in conjunction with progenitor nerve stem cells or mesenchymal cells to migrate under the retina to the central foveal area though the intercellular space and replace the retinal cell damage caused by age related macular degeneration or retinitis pigmentosa.

In one embodiment, the foveal drug or stem cell delivery to the center of the retina (fovea) is done without lifting the sensitive fovea by a novel injection in an area for 10-180 degrees of the superior retina through the sclera or through the vitreous cavity lifting only superior retina by the injection and leaving the sensitive fovea as “fovea sparing procedure” where the medications migrate by gravity through the retinal intercellular spaces to locate in the fovea without significantly lifting the fovea which would damage the fovea, e.g., in a patient with age-related macular degeneration or for gene delivery in various form of retinitis pigmentosa, or to cut and replace the mutated DNA or replace them with new gene using CRISPR-cas9, etc.

In one embodiment, the foveal drug or stem cell delivery or delivery of quantum dots, nanoparticles, or microparticles to the center of the retina (fovea) is done without lifting the sensitive fovea by a novel injection in an area for 10-180 degrees of the superior retina through the sclera or through the vitreous cavity lifting only the superior retina by the injection, while elevating the head and leaving the sensitive fovea as a “fovea sparing procedure” where the quantum dots migrate by gravity through the retinal intercellular spaces to locate in the fovea without significantly lifting the fovea, e.g., in patient with a genetic retinal disease, such as various forms of retinitis pigmentosa or Leber amaurosis congenital (LCA), etc., so that the disease process may be treated by gene delivery and CRISPR-Cas9, etc. to cut or replace the defective DNA or replace them.

In one embodiment, the foveal drug or stem cell delivery to the center of the retina (fovea) is done without lifting the sensitive fovea by a novel injection in an area for 10-180 degrees or more of the superior temporal retina through the sclera or through the vitreous cavity lifting only superior temporal retina by the injection and leaving the sensitive fovea as “fovea sparing procedure” where the medications migrate by gravity through the retinal intercellular spaces to locate in the fovea without significantly lifting the fovea which would damage the fovea, e.g., in a patient with genetic disease of the retina to inject piezoelectric nanoparticles or microparticles, that can be subsequently stimulated with ultrasound from outside to create an electric pulse in the retinal cells and initiate an action potential in the retinal cells that are in contact with the piezoelectric nanoparticles or microparticles to prevent their degeneration, since the retinal cells degenerate without an electric stimulation.

In one embodiment, the foveal drug or stem cell delivery to the center of the retina (fovea) is done along with administration of retinal stem cells or mesenchymal stem cells with a slow release polymeric nanoparticles carrying medication and one of Rock inhibitors, Wnt inhibitors, GSK inhibitors, or integrin inhibitors to prevent a postoperative inflammation for many months to years, or in combination with piezoelectric nanoparticles or microparticles where the injection is in the subretinal space through the sclera, or through the vitreous cavity under the subretinal space under microscopic observation, lifting only the desired retinal area by the injection and leaving the sensitive fovea untouched as a “fovea sparing procedure” where the medications migrate by gravity in the subretinal space or through the retinal intercellular spaces to locate in the fovea without significantly lifting the fovea which would damage the fovea, e.g., in a patient with genetic disease of the retina, etc.

In one embodiment, the quantum dot nanoparticles or piezoelectric nanoparticles and polymeric drug delivery nanoparticles for slow release medications are administered together or separately and injected only in the temporal or superior part of the retina to gradually penetrate the retina to reach the fovea through the intercellular spaces or subretinal spaces where the nanoparticles come into contact with the retinal ganglion cells and can be stimulated either with a non-toxic dose of a light source or ultrasound to generate electrical pulses to the retinal ganglion cells and other glial cells of the retina or the medications can be combined with gene therapy using CRISPR-cas9 with or without stem cell therapy to replace dead retinal cells or support retinal vasculature in diabetic retinopathy.

In one or more further embodiments, a method to enhance functional recovery of a cell in a patient in need thereof by administering functionalized, with or without anti-inflammatory compounds such as Rock inhibitors, graphene quantum dots, graphene-oxide quantum dots, graphene-zinc oxide quantum dots, graphene nanotubes, reduced graphene oxide and/or carbon nanotubes, gold nanoparticles, gold/iron nanoparticles, piezoelectric nanoparticles, such as quartz and perovskites, collectively termed nanoparticles, to a site in a patient where functional cell recovery is needed. Preferably, administration is performed using the plungerless aspiration and/or injection device described above. The nanoparticles at the site are controllably activated by light, thus controllably altering a cellular electrical property. Activation uses an internal device of a fiber optic comprising wires and a tip containing a light source, a sensor connectable to the fiber optic wires, and a controller to receive and generate electrical signals. Signals resulting from the altered cellular electrical property at the site are sensed and are optionally provided to a processor to monitor and/or controllably alter the electrical property using the controller. The processor may be implanted in the patient, e.g. under the skin, or may be external to the patient.

In one embodiment the sensor is an implanted graphene ribbon or nanoribbon, a wafer-scale epithaxically grown graphene on the surface of at least a portion of the fiber optic, acting as a transistor providing feedback to the controller, to which it is operatively connected, on the altered cellular electrical property. That is, the sensor monitors target cell electrical conditions and provides these to the controller, which in turn can modify control of the light based on the electrical conditions.

The light source may be a light emitting diode (LED) with a rechargeable battery. Ambient light, ultraviolet light, infrared light, or visible light may be used, and light exposure intensity and/or duration may be controlled. In one or more embodiments, the piezoelectric nanoparticles are stimulated non-invasively with ultrasound.

In one embodiment the nanoparticles are injected locally immediately prior to placement of the device through a cannula guided with magnetic resonance imaging (MRI).

The method may be used with neurons, muscle cells cardiac cells, ocular cells, etc.; on any cell that would benefit from such therapy. As an example, one candidate is a patient with a neural-related pathology, a neurodegenerative disease or symptom of such a disease, or blindness, and/or surgically injured neurons (e.g., patients after LASIK surgery and during LASIK surgery, prior to closing the corneal flap). Such patients include those with epilepsy, Parkinson's disease, Alzheimer's disease, depression, spinal cord injury, peripheral nerve injury, stroke, and chronic pain. The nanoparticles may be targeted or provided at a site of brain injury or spinal cord injury to controllably enhance neuronal growth. In one embodiment, the nanoparticles contain other agents to facilitate neuronal growth, e.g., Rock inhibitors which are anti-inflammatory and simultaneously inhibits TGF beta action on the tissue preventing fibrosis in the tissue or fibrillary entanglement in Alzheimer's disease, myelin basic protein (MBP), valproic acid, ketamine, donepezil hydrochloride, thymosin β10, thymosin α1, choline acetyl esterase, nerve growth factor (NGF), and/or brain derived growth factor (BDGF). As another example, one candidate is a patient with cardiac dysrhythmia, with the nanoparticles provided and controllably activated to control heart rate. Other agents may be included, e.g., stem cells, immunomodulators, anti-vascular endothelial growth factor (VEGF) agents, anti-integrin agents, anti-inflammatory agents, antibiotics, anti-viral agents, anti-fungal agents, anti-proliferative agents, and/or anti-cancer agents, also agents to enhance or impart biocompatibility.

One embodiment is a method for delivering an opsin family gene to an anatomical and/or physiological site for stimulating, modifying polarization of, and/or inducing an action potential in a cell at the site. This embodiment administers a complex comprising a non-quantum dot nanoparticle carrier, a biocompatible molecule for cell uptake of the complex, an opsin family gene or another gene with CRISPR/cas9 RNA, a biocompatible fluid, a Rock inhibitor which also inhibits TGF-β1/Smad2, 3 signal transduction, to the anatomical and/or physiological site, to result in formation of a light activated channel in a cell membrane permitting cell stimulation by an external or internal light transmitted by a fiber optic to modify polarization of, and/or induce an action potential in the cell at the site, and counteract an inflammatory response of the tissue to the disease process. Optional components include a targeting moiety such as an antibody, antigen, ligand, receptor, etc., and/or a second gene.

Examples of non-quantum dot nanoparticles include fullerenes, buckyballs, dendrimers, liposomes, aptamers, and/or micelles. Such non-quantum dot nanoparticles are known in the art, such as gold, iron graphene, graphene oxide organic and non-organic nanoparticles, or combinations of them with or without Rock inhibitors, etc. as only one non-limiting example, dendrimers include poly(amidoamine) (PAMAM), poly(amidoamine-organosilicon) (PAMAMOS), poly(propyleneimine) (PPIO), tecto, multilingual, chiral, hybrid, amphiphilic, micellar, multiple antipen peptide, and Frechet-type dendrimers. The nanoparticle can be functionalized to render or enhance its biocompatibility, and can be further treated or delivered to enhance cell penetration. To enhance biocompatibility, association with or covalent coupling to one or more of cell penetrating peptides (CPP), arginine-CPP, cysteine-CPP, polyethylene glycol (PEG), biotin-streptavadin, acetyl cysteine, an antibody, and/or a ligand for a receptor may be employed.

Opsin family gene members include rhodopsin, halorhodopsin, photopsin, and channelrhodopsin. The second gene is used where the patient has a genetic or acquired dengenerative disease or condition, where the second gene ameliorates at least to some extent the disease or condition by providing the component that is lacking, by enhancing gene activity, by silencing gene activity, etc., as known in the art. Examples include a degenerative retinal condition, a degenerative central nervous system disease, and a degenerative cardiovascular disease. The second gene may encode a protein, or it may be an inhibitory RNA (RNAi) for gene silencing.

The method may be used on an excitable cell, such as a retinal cell, a cardiac cell, a muscle cell, a central nervous system cell (CNS), a spinal cord cell, and/or a peripheral nerve cell. The method may also be used on a non-excitable cell, such as a fibroblast, a glial cell, a stem cell, and/or a pluripotential mesenchymal stem cell.

Light-induced cell stimulation may induce cell proliferation by inducing intermittent polarization and depolarization at frequencies 1-30 Hertz (1-30 cycles/sec) of the cell have a QD or nanoparticles and or opsin gene which is also activated with the same light enhancing the depolarization effect where the nanoparticles are conjugated with a Rock inhibitor which inhibits also TGF-β1/Smad2, 3 signal transduction, either in vitro in tissue culture, or in vivo, which desirably replaces cell loss due to various diseases or conditions at the targeted site.

In one embodiment, the examples of such conditions include, but are not limited to, age related macular degeneration, stroke, and ischemia, Alzheimer's disease, traumatic brain injury, Parkinson's disease, diabetic retinopathy, or neuropathy.

The functionalized nanoparticle/gene/Rock inhibitors complex may be administered by topical administration to conjunctiva, or other surface structure, such as nasal mucosa for the brain or an injection at an intraocular, intravitreal, intraretinal, subretinal, or intrathecal location or cerebrospinal fluid etc. Its entry or penetration into the target cell may be enhanced by focused ultrasound, electroporation, mechanical force or light energy. In one example, the complex is injected in a desired location and concentration with an insulated metallic needle connected to a power source for delivery of the complex inside the cell.

The complex may further contain a therapeutic agent, e.g., immunomodulators, an anti-inflammatory, e.g., Rock inhibitors such as Fasudil, Ripasudil, RKI-1447, Y-27632, GSK429286A, Y-30141, Simvastatin and combinations thereof, etc. that also inhibits the TGF beta, anti-VEGF agents, anti-integrins, anti-inflammatory agents, NSAIDs, antibiotics, anti-viral agents, anti-fungal agents, anti-proliferative agents, and/or anti-cancer agents.

In one embodiment, the complex is stimulated through the skin over the nose base using a laser light ultrasound.

One embodiment is a method for providing a gene to a target cell by administering to a patient in need thereof a plurality of functionalized nanoparticles, the nanoparticles coated with medication, such as Rock inhibitors, e.g., Fasudil (HA-1077), a selective RhoA/Rho kinase (ROCK) inhibitor, or Y-27632, small molecule inhibitor of ROCK1 and ROCK2, Ripasudil, RKI-1447, GSK429286A, Y-30141, and combinations thereof etc. comprising either a nanoparticle such as gold, iron, etc., plasmid or a liposome containing at least one G-protein and/or opsin-family gene or other genes attached to the nanoparticle and an antibody that targets the nanoparticles to a cell and coated with a biocompatible molecule for cell uptake, forming a complex of nanoparticle-plasmid-gene or the nanoparticle-liposome-gene, respectively, then stimulating the complex with an energy source under conditions sufficient to introduce the gene into the target cell. In this embodiment is plasmid or liposome is attached to the nanoparticles during a process of coating the nanoparticles with at least one PEG, PEI, chitosan, biotin, streptavidin, CPP, and/or ACPP. The gene attached to the nanoparticle and an antibody that targets the nanoparticles may be rhodopsin, holorodopsin, Go opsins, Gq opsins, photoisomerases, and/or neuropsins, CRISPR/cas9 may include DNA, RNA, and/or siRNA. The result is regulated membrane potential of the cells, induced action potential of the cells, and/or transmission of a signal from the cell to a second cell. Administration may be systemic or local, and optionally includes administration of at least one medicament. Local administration may be to the eye, central nervous system, peripheral nerves, and/or heart. In this embodiment, the complex is protected from degradation by the blood brain barrier and/or blood ocular barrier before reaching its intended tissue or cells. Administration may also be through the nasal mucosal by spraying, drops, or injection to access olfactory nerves and hence providing the complex to brain. The nanoparticle/gene complex is stimulated through the skin over the nose base using a laser light or ultrasound. For the method in general as described in this embodiment, the patient in need thereof may have, e.g., epilepsy, a mood disorder, post-traumatic stress disorder (PTSD), depression, fright, Parkinson's disease, Alzheimer's disease, a brain degenerative diseases, trauma, stroke, migraine headache, and/or an addiction. In one specific embodiment of the general embodiment, the complex is prepared in tissue culture of a cell type, e.g., neuronal, retinal, muscle, neurons, ocular cell, glial cell, and stem cell of any preceding cell type, prior to administering the complex to the patient. Stimulation of the complex may be by any of the following sources: visible light, ultraviolet light, infrared light, diode laser, ultrasound energy, and/or mechanical force. In a specific embodiment of the general embodiment, stimulation is by a processor as a light pulse applied to a site, e.g., the transfected organ, the heart as a pacemaker using a fiber optic implanted in the organ, externally for superficially located nerves, to the retina through the cornea or directly through the sclera, and/or to the brain through the nasal mucosa, where the processor optionally regulates the pulse number and/or duration, and the complex is stimulated through the skin over the nose base using a laser light or ultrasound. Application of light pulses to the transfected cells desirably causes an increase in the number of transfected cells in vivo or in vitro. Nanoparticles may be of any shape: e.g., spheres, nanotubes, nanowires, tetragons, hexagons, and/or cylinders. After cell transfection, the nanoparticles are removed by, e.g., cell expulsion, reticuloendothelial cell uptake, elimination in bile, elimination in sweat, elimination in urine, elimination in feces, etc. A specific embodiment also enhances tolerance of the nanoparticles in vivo. In this specific embodiment, the patient in need of treatment is administered magnetic nanoparticles, the magnetic nanoparticles excluding quantum dots, the nanoparticles comprising a plasmid or liposome containing at least one G-protein and/or opsin-family gene and an antibody that targets the nanoparticles to a cell and coated with a biocompatible molecule for cell uptake, forming a complex of nanoparticle-plasmid-gene. The complex is activated with an energy source. A localized magnet is applied, resulting in formation of a magnetic field at the complex site, with an electrostatic potential of the nanoparticles up to −25 mV. This magnetic field enhances penetration and tranfection of the gene into the cell. The method results in enhanced tolerance of the nanoparticles in vivo compared to quantum dots. In use, the complex is administered and a magnet is positioned at the desired transfection site to generate a magnetic field and attract the nanoparticles to the site. Administration may be, e.g., in the circulation, eye, CNS, peripheral nerves, and/or heart, and the magnet may be positioned, e.g., over the sclera behind the retina, frontal, parietal, posterior cortex, heart, spinal cord, peripheral nerves, and/or nose.

Combination mechanisms to correct, reduce, and/or prevent physiological electro-sensory damage or electromotor damage and promote functional recovery of excitable cells, e.g., neurons in the central nervous system (i.e., brain and spinal cord) and neuronal cells involved with visual, auditory, vocal, olfactory responses, e.g., retinal cells in the eye, cochlear cells in the ear, olfactory cells in the nose, etc., and neurons in the peripheral nervous system are provided. The inventive combination methods can be thought of as akin to combination approaches in treating neoplastic lesions, but targeting less than optimally-functioning excitable cells. The combination mechanism may also be used to correct, reduce, and/or prevent damage to tissues by rendering normally non-excitable cells in proximity to partially or wholly non-functional cells artificially functional.

In one embodiment, the combined method promotes functional recovery and controllably regulates plasma membrane polarization of a functional excitable neuronal cell. A biomolecule effecting gene therapy is administered into an eye and/or central nervous system of a patient in need of the therapy (e.g., a patient with a neuronal disease). Quantum dots and/or semiconductor nanowires (generically referred to hereafter as particles or solar cells) are administered into the eye and/or central nervous system of the patient, either simultaneously or sequentially either before or after the biomolecule is administered. Functionalized quantum dots are nanoparticulate semiconductors in which excitation is confined in all three spatial dimensions. Semiconductor nanowires are nanoparticulate semiconductors in which excitation is confined in two of the three spatial dimensions, with a nanoscale diameter but a length to width ratio of 100:1 or more. In one embodiment, all nanoparticles are functionalized and conjugated with Rock inhibitors. Semiconductor nanowires tend to be more efficient than quantum dots in converting electromagnetic radiation into electrical charge and more similar to solar cells in creating electromagnetic fields when stimulated by such radiation. In one embodiment, the particle comprises both a semiconductor and a metal, or two semiconductors, thus creating a hetero-junction, which together act as a photodiode. The difference in the chemical potentials of the two components, e.g., the semi-conductor and the metal, bends the energy bands of the semiconductor near the junction, creating a built-in electrical field. In one embodiment, the hetero-junction creates a Schottky junction, where illumination creates electron-hole pairs that separate under the influence of the built-in field, thereby yielding a photovoltage across the structure, e.g., the photovoltaic, or PV effect. Light is applied to the eye or central nervous system to controllably activate the particles by controlling exposure time, exposure intensity, exposure site, etc. to controllably regulate the plasma membrane polarization of the functional excitable neuronal cells and to provide the biomolecule to the neuronal cells. In one embodiment, the biomolecule is directly or indirectly associated with, or covalently conjugated to, the quantum dots and/or semiconductor nanowires so that in a single administration (e.g., one injection), both biomolecule and quantum dots components are provided to the patient. Once administered, the quantum dots can be imaged, tracked, monitoring, evaluated in the patient using a sensor or other tracking agent using methods well known in the art (e.g., digital imaging, etc.).

The light sensitive particles may be provided to specific neurons for therapy. As one example, they may be provided to an optic nerve for retinal therapy. As another example, they may be provided to an olfactory nerve for nasal nerve therapy, and/or as a point of entry for brain therapy, etc., the complex of the nanoparticle/gene is stimulated through the skin over the nose base using a laser light or ultrasound. As another example, they may be provided to selective or non-selective sites for selective stimulation of various regions, either alone or in combination. As non-limiting examples of selective stimulation of central nervous system nerves, the visual cortex can be stimulated through specific light stimulation of the retina, the olfactory neuron can be stimulated by smell, the auditory neuron can be stimulation by sound, etc., or piezoelectric nanoparticles may be stimulated with ultrasound energy. As non-limiting examples of selective stimulation of peripheral nervous system nerves, chronic pain may be controlled by direct stimulation of the appropriate nerves, and appetite may be suppressed by direct stimulation of appropriate nerves.

Stimulation by light may be achieved by several mechanisms, as known to one skilled in the art. For example, using activation of particles in the brain as an exemplary, non-limiting example, activation may be provided by a fiber optic device surgically placed at the desired area of the brain, located under the scalp, and illuminated by a light source, e.g., a light emitting diode (LED) through a small window made in the skull replaced by clear glass at a desired area. Such a window may remain hidden under the skin, because it is known that light can penetrate a few millimeters into skin. An analogous concept may be used for stimulating other areas of the central nervous system, the peripheral nervous system, or heart or other muscles, with or without application of a fiber optic device if quantum dots are injected through an opening into the superficial area of the brain, nerve, heart muscle, etc. Such stimulation may controllably regulate, i.e., activate/deactivate, by using an appropriate wavelength of light, or ultrasound non-invasively using ultrasound and piezoelectric nanoparticles, with or without a processor with the specific neuronal code as pulses. Quantum dots and/or semiconductor nanowires may be used in conjunction with stem cell therapy or in conjunction with other devices, e.g., prosthetic devices, that are activated or otherwise rely or light and/or electrical current.

In addition to using the method for the above indications and for treatment of retinal degeneration, etc. and posttraumatic epilepsy, the method also has applications in amelioration of the underlying pathology and/or symptoms of genetic and/or degenerative diseases, e.g., retinitis pigmentosa, retinal degeneration, diabetic retinopathy, traumatic brain injuries, central nervous system pathologies, such as Alzheimer's disease and Parkinson's disease, epilepsy, etc., dopamine-regulated disorders such as migraines, autism, mood disorders, schizophrenia, senile dementia, sleep disorders, restless leg syndrome, and depression. Tourette syndrome, restless leg syndrome, and stuttering are a part of the same spectrum of diseases characterized by malfunctioning membrane potential and electrical pulse transmission. The consequences of infectious diseases, epilepsy, paralysis, and traumatic injury of the brain and/or peripheral nerves are also amenable to therapy with the inventive method. All such disorders can be influenced either with particle administration alone or with particles associated with medication modifying cell membrane potential, e.g., carbonic anhydrase inhibitors. Amelioration includes any reduction in the signs, symptoms, and/or etiology, including but not limited to prevention, therapy, and curative effects, of any of the above indications. As one example, functionalized quantum dot nanoparticles, such as gold, graphene, etc. and/or semiconductor nanowires may be targeted to dopamine-regulated nerves for therapy of migraines, mood disorders, etc. As another example, quantum dots and/or semiconductor nanowires can be used for deep subthalamic, cerebral, or cortical and peripheral nerve stimulation for therapy of Parkinson's disease, etc.

A viral vector (e.g., adenovirus, adeno-associated virus, retrovirus) can provide the biomolecule, which can be a natural or synthetic protein, peptide, nucleic acid, oligonucleotide, gene, etc. when conjugated with the particles. However, viral vectors carry viral proteins, and can produce an immune response if repeated therapy is indicated. As such, in a preferred embodiment, a non-viral vector, such as gold, gold/iron nanoparticles or graphene nanocarbons, etc., connected to encoded DNA are used. In one embodiment, the biomolecule is a cell membrane ion channel protein such as rhodopsin, halorhodopsin, or other light-activated membrane ion channel protein. If the same wavelength of light stimulates both quantum dots and protein (or other biomolecule), the effect may be complementary and the result is an enhanced action potential in the excitable cells, i.e., this embodiment achieves a synergistic effect. If a different wavelength of light stimulates the quantum dots and protein (or other biomolecule), the result may be no action potential in the excitable cells, i.e., this embodiment achieves silencing of the action potential in the cell. It should be noted that the light energy should be of sufficient energy level and specific wavelength to stimulate QDs to induce an action potential in the cell membrane, therefore weak light pulses may not have sufficient energy to create a cell membrane depolarization and higher light power may reach a toxic level of 4000 lux. In one embodiment, the biomolecule, e.g., membrane channel protein, is excited by the same wavelengths of light that also excite the nanoparticle particles. In one embodiment, the biomolecule, e.g., membrane channel protein, is excited by a different wavelength of light than that exciting the particles, and then in turn its electrical field may not open the membrane channel protein. The variations can increase or reduce or suppress the action potential in the cell. In all cases, the “tunable” selection of the biomolecule and the particles, as well as the specific excitation energy (typically light but also ultrasound radiation energy can be used) applied, provides a controlled and regulated process. In turn, the selective on or off activation of the particles provides the high degree of control that enhances efficacy and safety and permits close monitoring and regulation.

Delivery and intercellular and/or intracellular and/or intramembrane localization of nano- and micro-particle solar cells within and/or among excitable biological cells to regulate membrane polarization of biological cells combined with other methods to promote functional recovery of damaged excitable cells in the eye and central nervous system. The inventive method provides solar cells in a minimally invasive procedure into the eye, heart, and/or the central nervous system; the solar cells are not implanted in the body in an invasive procedure. The inventive method provides a plurality of solar cells as discrete individual particles; the solar cells are not connected as a unit and do not have a backing layer or backing material. The inventive method uses solar cells that may be activated by ambient light; the method does not use an electrical apparatus and thus does not use photodiodes, stimulating electrodes, or other electrical devices. The inventive method uses solar cells to enhance the regulation of polarization by the excitable biological cells themselves; the solar cells facilitate or boost the ability of excitable biological cells to normalize or regulate their own polarity. In one embodiment, the inventive method provides for excitable biological cells to regulate their own polarity; stimulation of the solar cells used in the invention does not generate an action potential to regulate polarity, but instead facilitates the biological cells themselves to regulate polarity. In one embodiment, the inventive method provides for stimulation of the solar cells used in the invention to generate an action potential. The inventive method provides semiconductor particles in combination with gene therapies to enhance functional recovery of neuronal cells damaged by different etiologies, including genetic disorders, ischemic or vascular damage, and age-related damage. By combining modulation of cell polarization, which takes advantage of the ability to regulate quantum dots and/or semiconductor nanowires, with genetic and other approaches to therapy, neuronal degenerative process are ameliorated.

Biological cells are bound by a plasma membrane. In all cells, this membrane has a resting potential. The resting potential is an electrical charge across the plasma membrane of the non-excited or resting cell, rendering the interior of the cell negative with respect to the exterior. Hence, the plasma membrane of all biological cells in their resting state is polarized.

The extent of the resting potential varies among different cell types. In cells such as nerve, muscle, and retinal cells, which are excitable in that they can be stimulated to create an electric current, the resting potential is about −70 millivolts (mv). This resting potential arises from two components of the plasma membrane: the sodium/potassium ATPase, which pumps two potassium ions (K+) into the cell for every three sodium ions (Na+) it pumps out of the cell, and “leakiness” of some K+ channels, allowing slow facilitated diffusion of K+ out of the cell. The result is a net loss of positive charge from within the resting cell.

Certain external stimuli reduce the charge across the plasma membrane, resulting in membrane depolarization. As one example, mechanical stimuli (e.g., stretching, sound waves) activate mechanically-gated Na+ channels. As another example, certain neurotransmitters (e.g., acetylcholine) open ligand-gated Na+ channels. In each case, the facilitated diffusion of Na+ into the cell depolarizes the membrane; it reduces the resting potential at that membrane location. This creates an excitatory postsynaptic potential (EPSP).

If the potential at any membrane location is reduced to the threshold voltage, many voltage-gated Na+ channels open in that location, generating an influx of Na+. This localized, sudden, complete depolarization opens adjacent voltage-gated Na+ channels. The result is a wave of depolarization along the cell membrane, referred to as the action potential or, in excitable cells, an impulse.

A second stimulus applied to an excitable cell within a short time (less than 0.001 second) after the first stimulus will not trigger another impulse. This is because the membrane is depolarized, leaving the cell in a refractory period. Only when the −70 mv polarity is reestablished, termed repolarization, will an excitable cell be able to respond to another stimulus. Repolarization is established by facilitated diffusion of K+ out of the cell. When the cell is finally rested, Na+ that entered the cell at each impulse are actively transported back out of the cell.

Hyperpolarization occurs when negatively charged chloride ions (Cl−) enter the cell and K+ exit the cell. Some neurotransmitters may facilitate this by opening Cl− and/or K+ channels in the plasma membrane. Hyperpolarization results in an inhibitory postsynaptic potential (IPSP); although the threshold voltage of the cell is unchanged, it requires a stronger excitatory stimulus to reach threshold.

Abnormal cell polarization may affect regenerative and/or functional process of excitable cells, and result in cell dysfunction. Abnormal cell polarization includes, but is not limited to, any of the following and whether transient or sustained: loss of polarization, decreased polarization, altered polarization, hyperpolarization, and any deviation from normal cell polarization. Excitable cells include, but are not limited to, sensory cells (e.g., retina and macula of the eye), neuronal cells in the central nervous system (CNS) (brain and spinal cord) and peripheral nervous system, muscle cells (striated, cardiac, and smooth muscle cells).

The orientation of the cell with respect to its apical, lateral, and basal surfaces may affect polarization and may be regulated by the inventive method. Adjacent cells communicate in the lateral domain, with attachment or contact sites by which cells adhere to one another. Terminal bars, attachment sites between cells that act as a barrier to passage of substances, are located around the entire circumference of cells and are composed of junctional complexes responsible for joining individual cells. Occluding junctions, also referred to as tight junctions or zonula occludentes, are located apically within the lateral domain and encircle the cell, separating the luminal region from the intercellular space and cytoplasm. These are narrow regions of contact between the plasma membranes of adjacent cells and seal off the intercellular space, forming an impermeable diffusion barrier between cells and preventing proteins from migrating between apical and lateral surfaces of the cell. In one embodiment, the method selectively regulates polarization in areas of the cell bound by occluding junctions. Particles may be selectively positioned and/or selectively regulated to regulate polarization at a desired site.

Ischemic cell death is caused by failure of the ionic pumps of the plasma membrane. Depolarization of the plasma membrane in retinal cells and subsequent synaptic release of L-glutamate are implicated in ischemic retinal damage. However, lasting depolarization of any cell will interfere with the normal cell membrane potential, and subsequently all the physiological cell function, and will lead to cell death. Mali et al. (Investigative Ophthalmology and Visual Science, 2005, 46, 2125) reported that when KCl, a known membrane depolarizing agent, is injected into the vitreous humor, the subsequent membrane depolarization results in a dose- and time-related upregulation of matrix metalloproteinase (MMP)-9 activity and protein in the retina. This was associated with an increase in proapoptotic protein Bax and apoptotic death of cells in the ganglion cell layer and inner nuclear layer, and subsequent loss of NF-L-positive ganglion cells and calretinin-positive amacrine cells. A synthetic MMP inhibitor inhibited KCl-mediated MMP-9 upregulation, which led to a significant attenuation of KCl-induced retinal damage. Regulating polarization thus inhibits MMP-9 and decreases damage that can diminish visual acuity.

Methods to regulate membrane polarization of excitable cells assist in minimizing physiologic damage and reducing pathology including but not limited to ischemic damage to the retina, degenerative diseases of the retina including but not limited to retinitis pigmentosa, ischemic and/or degenerative diseases of cardiac muscle, and/or ischemic and degenerative diseases of cerebral tissue, etc. In turn, the method minimizes or prevents undesirable effects such as loss of visual acuity, myocardial infarction, cerebral stroke, etc. and enhances a patient's quality of life.

Only the cells involved with vision, such as retinal photoreceptors have opsin gene channels (i.e., rhodopsin/halorhdopsin gene, etc.) in their membranes and are able to respond to the light by polarization and depolarization of their cell membrane and initiating potentially an action potential by transmitting an electrical signal to the next cells.

Cells lacking opsin genes are mesenchymal cells, smooth muscle cells, cardiac muscle cells, and cranial and peripheral nerve cells. Although, the nerve cells may respond to chemical or electrical or sound stimuli.

The photoreceptor cells may not respond to light stimuli with a polarization/depolarization effect because of either a genetic disease affecting the opsin gene or loss of other genes affecting the normal physiological function of the photoreceptor cell. The weakness of cell membrane channels, such as sodium, potassium, calcium, and chloride channels can also explain the weakness of photoreceptor response to a light stimulus.

In short, a full response to a weak light stimuli requires a normal functioning opsin gene and membrane channel genes. This concept is specifically important in cells that do not normally possess opsin gene even though they are excitable such as nerve cells or cardiac cells. Transfecting these cells with rhodopsin or halorhodopsin gene will require a strong light stimulus to excite the channel rhodopsin that causes light toxicity.

In one embodiment, the administration of the carrier nanoparticles and their cargo replaces or strengthen at least three or more genes along with CRISPR/cas9 in the cells, intended to respond to light or any other stimulus such as ultrasound inducing depolarization/polarization or an action potential in the cell membrane.

In one embodiment, the application of this using QDs and one or more opsin genes in growing cells or stem cells stimulates them to grow and multiply under the control of a light pulses inducing polarization and depolarization and stimulating subsequently cell division in vitro and in vivo where these cells are injected or pre-existing cells, such as tissue stem cells, neuronal stem cells or glial stem cells, where pulses of 30 per second frequencies stimulate the cell multiplication. In this embodiment, the gene transfer is done using nanoparticles/quantum dots, organic or non-organic metallic or non-metallic nanoparticles, dendrimers, fullerenes, of any size or form and conjugated to the desired DNA for administration. The nanoparticles are functionalized to become more biocompatible and conjugated with cell penetrating peptides to enhance cell penetration. The cells can be animal cells or plant cells. Simultaneously other compounds, such as Rock inhibitors, gene or medications are administered to enhance cell function, e.g., in neuronal cells. or enhance growth or treat the existing conditions, such as inflammation in Alzheimer's disease or diabetic retinopathy.

Methods to regulate membrane polarization of cells may also be used to create analogs to excitable cells from target cells that under normal physiologic conditions do not respond to the same stimuli. This embodiment beneficially preserves at least partial, if not substantially complete or complete, function of the overall tissue. For example, because particles such as quantum dots and/or semiconductor nanowires can be inserted into cell membranes and/or pass through cell membranes, the particles and/or nanowires can convert target cells that normally lack significant levels of rhodopsin, e.g., mesenchymal cells, glial cells, etc., into cells that are able to respond to certain wavelengths of light through hypo- or hyperpolarization. In one embodiment, the particles and/or nanowires may be conjugated with agents that stimulate or suppress the production of light-stimulated cell membrane ion channel proteins to influence the target cell's response to light. In one embodiment the agent is a gene encoding a channelrhodopsin protein. In one embodiment the particles and/or nanowire may be conjugated with agents such as nucleic acids or oligonucleotides that direct production of membrane ion channel proteins to make target cells excitable by stimuli such as wavelengths of light (e.g., retinal cells), mechanical vibration (e.g., cochlear cells), small molecules (e.g., olfactory cells), etc. In one embodiment the nucleic acids or oligonucleotides are regulatory sequences that stimulate transcription of genes encoding such regulatory proteins. In one embodiment the nucleic acids or oligonucleotides are sequences that encode such proteins.

Methods to regulate membrane polarization may also be used to modify stem cells for transplantation within the patient tissue. Autologous stem cells treated with particles and/or nanowires may be cultured and used to repopulate cells lost or destroyed in degenerative diseases of the retina, brain, heart, etc.

In one embodiment, therapeutic stimulation of the particles, such as stimulating graphene with light, counteracts or delays the effects of the underlying disease process by direct stimulation of the cells. As previously described, modulation of cell plasma membrane polarization may minimize physiologic damage and reduce pathology in the repopulated cells.

In one embodiment autologous stem cells treated with particles conjugated with genes and/or gene therapy vectors may be used to both deliver gene therapy and label the modified stem cells. After providing to patient tissues, the quantum dots and/or semiconductor nanowires may be imaged, tracked, monitored, regulated, and evaluated in the patient for cell survival and maturation rates, treatment efficacy, etc.

In one or more embodiments, the administration of the nanoparticle carriers with their cargo replaces or strengthen at least three or more genes along with CRISPR/cas9 in the cells, intended to respond to light or any other stimulus such as ultrasound inducing depolarization/polarization or an action potential in the cell membrane in growing neuronal or glial cells or stem cells in vitro or in vivo stimulates them by inducing polarization and depolarization, and to multiply under the control of a light pulses in vitro and in vivo.

In one embodiment, the particles and/or nanowires may be adapted to respond to electromagnetic radiation by emitting fluorescence radiation and the distribution and/or state of the nanoparticles and/or nanowires may be evaluated using a fluorescence microscope emitting the appropriate wavelength of light to activate the particles. In one embodiment, autologous stem cells treated with particles linked to magnetic nanoparticles may be used to both label stem cells and provide directional bias to the cells.

In one embodiment, the gold nanoparticles particles and/or combination nanowires and magnetic/gold nanoparticles may be conjugated with natural or synthetic biomolecules, e.g., proteins, peptides, nucleic acids, oligonucleotides, etc., that bind to specific locations in and/or on a cell and, after administration to a patient, may be subjected to a magnetic field applied outside the tissue, e.g., by permanent magnets temporarily affixed to the body in proximity to the eye, brain, heart, etc., to provide a predetermined directionality to the cells through attraction to the magnetic field. The particles may be made biocompatible by coating them with a biocompatible polymer such as (poly)ethylene glycol (PEG) moieties. Various biomolecules may be conjugated to one or the other or both of the particles and linked magnetic nanoparticles to cause them to bind to different locations in and/or on the treated cells.

In one embodiment, the nanoparticles, such as a semiconductor-metal particle, can be coated such that the nanoparticle is amphiphilic, where a portion of the nanoparticle is rendered hydrophilic and another portion of the nanoparticle is rendered hydrophobic. In one embodiment, and using a CdSe/Au particle as an example, the CdSe/Au particles are covered by trioctylphosphine oxide and alkylphosphonic acid, both of which are hydrophobic. Surface functionalization covers the Au portion of the CdSe/Au particles with polyethylene glycol, making them hydrophilic; the CdSe portion, still covered by trioctylphosphine oxide and alkyl phosphonic acid, remains hydrophobic. In one embodiment, CdSe/Au particles are suspended in N,N-dimethylformamide containing detergent (e.g., 1% Triton X-100) and exposed to polyethylene glycol-(CH2)10-SH to coordinate the thiol to the Au end.

Such amphiphilic particles may be inserting into cell membranes with the hydrophobic portion of the particle embedded within the cell membrane and the hydrophilic portion of the particle exposed to the intracellular and/or extracellular space. Alternatively, the hydrophobic portion may associate with the inner and/or outer surface of the cell membrane. In embodiments, the amphiphilic particles may be incorporated into micelles or liposomes, using methods known in the art, and the particle-containing liposome or micelle can be administered to a patient. After incorporation of amphiphilic particles into a bilayer membrane of a liposome, assimilation of the liposome into a cell membrane delivers the particle into the membrane, with the hydrophobic portion immersed in the lipid portion of the membrane, and the hydrophilic extending into the aqueous phase. The liposome or micelle may contain additional biomolecules, e.g., targeting moieties such as antibodies, cell surface receptors, etc., as well as additional therapeutic agents.

The inventive method may be more fully appreciated with respect to its utility in a single organ, such as the eye or brain. One skilled in the art will realize, however, that it is not so limited and is applicable to other cells.

In one embodiment, the inventive method externally administers to a patient a composition or, alternatively a device in a biocompatible composition, comprising functionalized particles and/or nanowires or solar cells to stimulate the cell membranes from inside of the cell or outside of the cell or within the cell membrane of all retinal cells. In one embodiment, the quantum dots and/or semiconductor nanowires are applied topically or injected into the eye and are delivered to the retinal cell cytoplasm or nucleus or cell membrane. In one embodiment, the quantum dots and/or semiconductor nanowires injected into the eye and are delivered into the cell membrane of retinal ganglion cells. In one embodiment, the quantum dots and/or semiconductor nanowires are introduced into the central nervous system by topical application to the nose or injected in the cerebrospinal fluid, but stimulated through the mucosa or outside the nose through the skin using a laser light or ultrasound when the nanoparticles are piezoelectric.

In one embodiment, the quantum dots and/or semiconductor nanowires are conjugated or otherwise associated with proteins or other moieties, such as Rock inhibitors, and provided using a vector to a patient to effect functional recovery of neuronal cells. One non-limiting example of this embodiment is quantum dots conjugated with a channel proteins introduced via a viral vector (e.g., adeno-associated virus (AAV)) to effect retinal gene therapy. Such a vector and/or quantum dots can be labeled for visualization, tracking, sensing, etc. For example, the quantum dots can be labeled or tagged with a signal recognition moiety. Such a vector can incorporate quantum dots into the viral capsid using, e.g., (poly)ethylene glycol (PEG) moieties. Another non-limiting example is the use and selective regulation, selective activation/deactivation alone or in combination, to monitor interfering RNA (RNAi) delivery and regulate gene silencing. Another non-limiting example is the use of quantum dots for in situ visualization of gene expression. This may be performed using quantum dot-DNA-coated polymer.

Another non-limiting example is the use of selective regulation, selective activation/deactivation alone or in combination, to monitor interfering RNA (RNAi) delivery and regulate gene silencing. Yet another non-limiting example is the use of quantum dots for in situ visualization of gene expression. This may be performed using a quantum dot-DNA-coated polymer. Semiconductor nanowires, nanocages, nano gold or piezoelectric nanoparticles, such as quartz, perovskites, etc. may be used in place of, or in addition to quantum dots or gold nanoparticles in each of these examples. Combinations of these embodiments are contemplated and included, using methods known by one skilled in the art. In general, the nanoparticles, such as gold or graphene, are used as vectors for gene modification as non-homologous end joining or homology directed repair (HDR), where the nanoparticles are coated with a cell penetrating agent or a donor DNA and a CRISPR/cas9 guide RNA used along with a thermal energy source that heats up the gold nanoparticles and enhances the cell penetration and endosomal escape while the DNA is not damaged because it is heat resistant as non-homologous end joining or homology directed repair (HDR).

Semiconductor nanowires may be used in place of or in addition to quantum dots in each of these examples. Combinations of these embodiments are contemplated and included, using methods known by one skilled in the art and as subsequently described.

As used herein, particles, quantum dots, and solar cells are used synonymously.

The retinal cells comprise at least ganglion cells, glial cells, photoreceptor cells, Muller cells, bipolar cells, horizontal cells, microglial cells, and cells of the neural fibers, etc. The amount of stimulation, or degree of membrane stimulation, can be regulated by the amount of energy provided by the particles. The total amount of energy provided by the particles to transmit to the membrane depends upon the time of particle activation.

The particles are activated by the energy source; the response to the specific wavelength depends on the inner material building the inner semiconductor. The energy source to activate the particles provides ambient light, ultraviolet light, visible light, infrared light, or ultrasound radiation.

In one embodiment, the quantum dots (QDs) are stimulated with an appropriate wavelength of light from ultraviolet to infrared, or ultrasound to induce the membrane channels of the cells to open and become more accessible for the penetration of the nanoparticles inside the cell and gene delivery and escape from the endosomes.

In one embodiment, the particles respond to blue, red, green, or IR light. In one embodiment, a plurality of particles respond to various specific wavelengths. In one embodiment, the particles have multiple semiconductor cores, and thus respond to various wavelengths. The wavelength selections are photons with different energies. The particles must have energy bandgaps or energy statues that match the energy of the photons. One skilled in the art tunes the energy levels using materials with different band-gaps or by carefully selecting the quantum size as it effects the energy level. Thus, one uses different size particles and/or particles with different cores. In one embodiment, the activation time interval ranges from 1 second to 100 seconds.

The source of energy activates the particles for the particles to provide sufficient energy to activate the membrane. In one embodiment, the energy source sufficient to activate the particles ranges from about one picojoule to one microjoule. In one embodiment, the activation energy source is external ambient light. In one embodiment, the activation energy source is a diode, LED, etc. Other activation energy sources are possible, as known by one skilled in the art. The energy source provides electromagnetic radiation, as known to one skilled in the art. Electromagnetic radiation includes infrared radiation (700 nm to 1 mm), visible light (380 nm to 760 nm), and ultraviolet radiation (4 nm to 400 nm), or alternatively, stimulation may be by means of ultrasound in case of piezoelectric nanoparticles such as quartz, perovskites, etc. The energy source is varied to vary the response of the particles; as one skilled in the art is aware, the shorter the wavelength, the more energy is delivered. As an example, infrared wavelengths (thermal activation), visible and ultraviolet wavelengths are provided for activating the particles to produce the desired photovoltaic (energy) response from the particle by a separate energy source or one that can provide combinations of the required wavelength ranges. The energy source(s) may be externally programmed (such as by computer software) to produce different wavelengths resulting in photovoltaic responses at desired time intervals. The regulation or control of the timed production of generated photovoltaic responses from the particles can be used to control the regulation of cell membrane potentials. The energy input from the energy source may be varied to vary the particles responses, hence regulating and/or controlling the membrane potential. The particles respond to the specific wavelength(s) to which they are exposed. A specific coating to the particles renders them specific. The protein coating can direct them to attach to certain cell membranes, and/or to enter a cell such as a normal cell, a tumor cell, a nerve cell, a glial cell, The particles, albeit relatively non-selective, can potentially increase the membrane potential of any cells to which they come into contact. After exposure to light, a diode, etc. they emit an electrical potential, current, or fluorescence. The electrical potential generated by this exposure to radiation increases the cell membrane potential. In an example of a specific application, a particle may be adapted to bind a photoreceptor of the eye and to trigger a hyperpolarization of the photoreceptor in response to activation by infrared light. The administration of such a particle may enable a patient to visually perceive at least some sources of infrared radiation, i.e., to have a ‘night vision’-like visual perception.

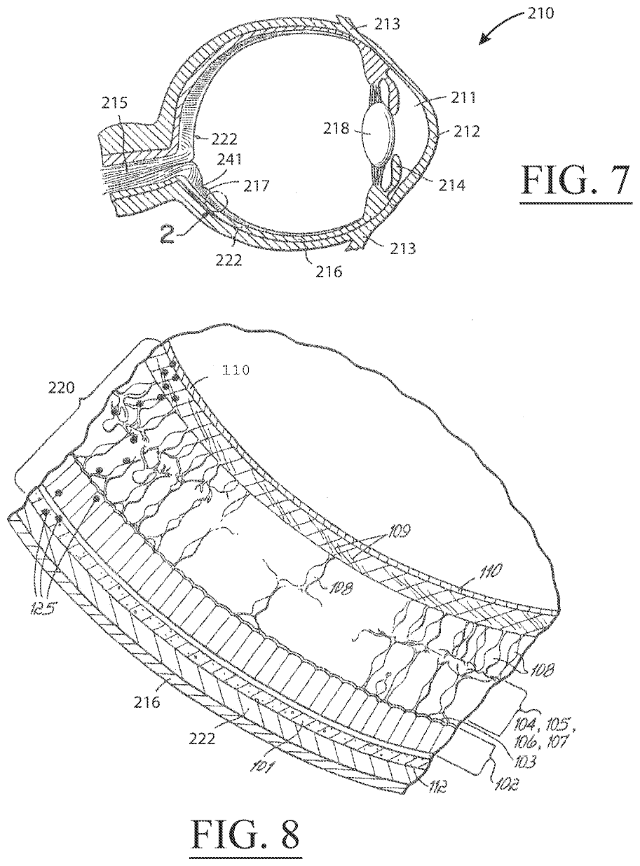

FIG. 7 shows a mammalian eye 210. The structures and locations of the anterior chamber 211, cornea 212, conjunctiva 213, iris 214, optic nerve 215, sclera 216, macula lutea or macula 217, lens 218, retina 220, choroid 222, and fovea 241 are indicated. The macula is located in the center of the posterior part of the retina 220 and is the most sensitive portion of the retina. It is an oval region of about 3 mm by 5 mm, in the center of which is a depression, the fovea centralis 241, from which rods are absent. Inside the fovea 241 is the point of entrance of the optic nerve 215 and its central artery. At this point, the retina 220 is incomplete and forms the blind spot.

The encircled area 2 of FIG. 7 is shown in exploded form in FIG. 8. As shown in FIG. 8, the retina 220 forms the innermost layer of the posterior portion of the eye and is the photoreceptor organ. The retina 220 has an optical portion that lines the inner surface of the choroid 222 and extends from the papilla of the optic nerve 215 to the ora serrata anteriorly. At the papilla, where the retina 220 stops, and at the ora serrata, the retina 220 is firmly connected with the retinal pigment epithelium (RPE) 101.

The retina 220 has ten parallel layers. These are, from the choroid in, as follows: the RPE 101, photoreceptor cells (rod and cone inner and outer segments) 102, the external limiting membrane 103, the outer nuclear layer 104, the outer plexiform layer 105, the inner nuclear layer 106, the inner plexiform layer 107, the layer of ganglion cells 108, the layer of optic nerve fibers or neurofiber layer 109, and the internal limiting membrane 110. The internal limiting membrane 110 is very thin (less than 5 μm), and normally adheres with the neurofiber layer 109 of the ganglion cells 108.

The pigment epithelial cell layer or RPE 101 rests on a basal lamina termed Bruch's membrane 112 that is adjacent to the choroid 222.

The next three layers are composed of various portions of one cell type, termed the first neuron. These layers are the photoreceptor region (lamina) 102 of rods and cones, the external limiting membrane 103, and the outer nuclear layer 104 composed of the nuclei of the rods and cones cells. The rods have long, thin bodies, and the cones have a broad base. The rods have greater sensitivity for low light levels; the cones have better visual acuity in daylight and are also responsible for color perception. There are three types of cones, each absorbing light from a different portion of the visible spectrum: long-wavelength (red), mid-wavelength (green), and short-wavelength (blue) light. Both rods and cones contain the transmembrane protein opsin, and the prosthetic group retinal, a vitamin A derivative. The opsins in each cell type contain different amino acids that confer differences in light absorption.

The RPE, photoreceptor cells, external limiting membrane, outer nuclear layer, and outer plexiform layer constitute the neuro-epithelial layer of the retina.

The inner nuclear layer, inner plexiform layer, ganglion cell layer, nerve fiber layer, and internal limiting membrane constitute the cerebral layer of the retina. The inner nuclear layer contains bipolar cells, ganglion cells, horizontal cells, amacrine cells, Muller cells, and astrocytes, the latter two being types of glial cells. The Muller cells have nuclei in the inner nuclear area and cytoplasm extending from the internal limiting membrane 110 to the external limiting membrane 103. The external limiting membrane 103 is a region of terminal bars between Muller's cells and the visual receptors.

The next three layers of the retina are composed of various parts of the second neurons, whose nuclei reside in the inner nuclear layer and whose cytoplasmic processes extend into the outer plexiform layer to synapse with the receptor cells and to the inner plexiform layer to synapse with the ganglion cells. Thus, the second neuron is bipolar.

The third neuron, the multipolar ganglion cells, sends its nerve fiber (axon) to the optic nerve.

The last layer of the retina is the internal limiting membrane (ILM) on which the processes of the Muller's cells rest.

The retina contains a complex interneuronal array. Bipolar cells and ganglion cells are sensory cells that together form a path from the rods and cones to the brain. Other neurons form synapses with the bipolar cells and ganglion cells and modify their activity. For example, ganglion cells, or ganglia, generate action potentials and conduct these impulses back to the brain along the optic nerve. Vision is based on the modulation of these impulses, but does not require the direct relationship between a visual stimulus and an action potential. The visual photosensitive cells, the rods and cones, do not generate action potentials, as do other sensory cells (e.g., olfactory, gustatory, and auditory sensory cells).

Muller cells, the principal type of glial cells, form architectural support structures stretching radially across the thickness of the retina, and forming the limits of the retina at the outer and inner limiting membranes, respectively. Muller cell bodies in the inner nuclear layer project irregularly thick and thin processes in either direction to the outer and inner limiting membranes. These processes insinuate themselves between cell bodies of the neurons in the nuclear layers, and envelope groups of neural processes in the plexiform layers. Retinal neural processes can only have direct contact, without enveloping Muller cell processes, at their synapses. The junctions forming the outer limiting membrane are between Muller cells, and other Muller cells and photoreceptor cells, as sturdy desmosomes or zonula adherens. Muller cells perform a range of functions that contribute to the health of the retinal neurons. These functions include supplying end-products of anaerobic metabolism (breakdown of glycogen) to fuel neuronal aerobic metabolism; removing neural waste products such as carbon dioxide and ammonia and recycling spent amino acid transmitters; protecting neurons from exposure to excess neurotransmitters using uptake and recycling mechanisms; phagocytosis of neuronal debris and release of neuroactive substances; synthesizing retinoic acid, required in the development of the eye and nervous system, from retinol; controlling homeostasis and protecting neurons from deleterious changes in their ionic environment by taking up and redistributing extracellular K+; and contributing to generation of the electroretinogram (ERG) b-wave, the slow P3 component of the ERG, and the scotopic threshold response (STR) by regulating K+ distribution across the retinal vitreous border, across the whole retina, and locally in the inner plexiform layer of the retina.

Astrocytes, the other type of glial cell, envelope ganglion cell axons and have a relationship to blood vessels of the nerve fiber, suggesting they are axonal and vascular glial sheaths and part of a blood-brain barrier. They contain abundant glycogen, similar to Muller cells, and provide nutrition to the neurons in the form of glucose. They may serve a role in ionic homeostasis in regulating extracellular K+ levels and neurotransmitter metabolism. They have a characteristic flattened cell body and fibrous radiating processes which contain intermediate filaments. The cell bodies and processes are almost entirely restricted to the nerve fiber layer of the retina. Their morphology changes from the optic nerve head to the periphery: from extremely elongated near the optic nerve to a symmetrical stellate form in the far peripheral retina.

Microglial cells are not neuroglial cells and enter the retina coincident with mesenchymal precursors of retinal blood vessels in development, and are found in every layer of the retina. They are one of two types. One type is thought to enter the retina at earlier stages of development from the optic nerve mesenchyme and lie dormant in the retinal layers for much of the life of the retina. The other type appears to be blood-borne cells, possibly originating from vessel pericytes. Both types can be stimulated into a macrophagic function upon retinal trauma, in degenerative diseases of the retina, etc. when they then engage in phagocytosis of degenerating retinal neurons.

All glial cells in the central nervous system (CNS) are coupled extensively by gap junctions. This coupling underlies several glial cell processes, including regulating extracellular K+ by spatial buffering, propagating intercellular Ca2+ waves, regulating intracellular ion levels, and modulating neuronal activity.

Activation of retinal glial cells with chemical, mechanical, or electrical stimuli often initiate propagated waves of calcium ions (Ca2+). These Ca2+ waves travel at a velocity of 23 m/second and up to 180 m/second from the site of initiation. The waves travel through both astrocytes and Muller cells, even when the wave is initiated by stimulating a single astrocyte.

Ca2+ waves propagate between glial cells in the retina by two mechanisms: diffusion of an intracellular messenger through gap junctions, and release of an extracellular messenger. Ca2+ wave propagation between astrocytes is mediated largely by diffusion of an intracellular messenger, likely inositol triphosphate (IP3), through gap junctions, along with release of adenosine triphosphate (ATP). Propagation from astrocytes to Muller cells, and from one Muller cell to other Muller cells, is mediated by ATP release.

Retinal neurons and glial cells also communicate. Muller cells have transient Ca2+ increases that occur at a low frequency. Stimulating the retina with repetitive light flashes significantly increases the frequency of these Ca2+ transients, most prominent in Muller cell endfeet at the retinal surface, but also in Muller cell processes in the inner plexiform layer. This neuron-to-glial cell communication indicates that glial cell Ca2+ transients are physiological responses in vivo.

Stimulated glial cells directly modulate the electrical activity of retinal neurons, leading either to enhanced or depressed neuronal spiking. Inhibitory glial modulation of neuronal spiking may be Ca2+-dependent, because the magnitude of neuronal modulation was proportional to the amplitude of the Ca2+ increase in neighboring glial cells. Glial cells can modulate neuronal activity in the retina by at least three mechanisms. In some ganglion cells, glial cell activation facilitates synaptic transmissions and enhances light-evoked spiking. In other ganglion cells, there is depressed synaptic transmissions and decreased spiking. Glial cell activation can also result in ganglion cells hyperpolarization, mediated by activating A1 receptors and opening neuronal K+ channels.

Stimulated glial cells also indirectly modulate the electrical activity of retinal neurons. This is mediated by glutamate uptake by Muller cells at synapses by glutamate transporters such as GLAST (EAAT1) and GLT-1 (EAAT2) in Muller cells. When glutamate transport in the retina is blocked, both the amplitude and the duration of ganglion cell EPSCs are increased. Glial cell modulation of electrical activation of retinal neurons is also mediated by regulating extracellular K+ and H+ levels. Neuronal activity leads to substantial variations in the concentration of K+ and H+ in the extracellular space, which can alter synaptic transmission; an increase of K+ depolarizes synaptic terminals, while an increase of H+ blocks presynaptic Ca2+ channels and NMDA receptors. Muller cells regulate extracellular concentrations of K+ and H+, thus influencing the effect of these ions on synaptic transmission.

With reference to FIG. 8, one skilled in the art will appreciate that solar cell micro- and/or nano-particles 125, provided selectively or substantially throughout the all regions of the retina, enhance, facilitate or boost the ability of these biological cells to regulate their polarity. This is in contrast to use of a device that supplies an electrical potential, that is implanted in an invasive surgical procedure, that is localized, etc. In embodiments solar cell micro- and/or nano-particles 125 may be provided in combination with implanted light guides, such as fiber optics, to enhance the efficiency of therapeutic stimulation. The micro- and/or nano-particles 125 may be coated with or, if the light guide material includes a polymer, included in at least a surface layer of guides having conventional cylindrical shapes, tubular shapes, substantially two-dimensional shapes, or three-dimensionally-branching tree-like structures. As one example, an implanted guide structure coated with the particles and membrane ion channel activators may be implanted inside any layer of the eye (e.g., subretinally, intraretrinally, epiretinally, in the vitreous, in the choroid, etc.) and activated with light to stimulate specific layers of cells. As another example, injected particles may be stimulated by implanted guide structures with light at lesser intensities than would be required by purely transmissive exposure from an entirely extra-ocular source.

Besides pathologies in one or more of the above described mechanisms to maintain and/or regulate retinal cell polarity, other excitable cells besides the retina may have pathologies that occur from defects in cell plasma membrane polarization. As one example, excitable cells in the brain of Alzheimer's patients have abnormal electrical conducting and stabilizing mechanisms, resulting in loss of electrical stimulation. Repolarization of these cells provides additional stimulation to replace the abnormal cell membrane polarization and/or the cell membrane polarization that was diminished or lost. As another example, glial cell scar tissue culminating from epileptic seizures results in abnormal electrical stabilizing mechanisms in excitable cells of the brain. Repolarization of these cells provides a stabilized threshold, resulting in a calming effect. One skilled in the art will appreciate other pathologies for which the inventive method may be used. Therapeutic stimulation of the brain, spinal cord, and/or peripheral nerves may similarly be performed with implanted fiber optics, including cylindrical, tubular, substantially two- or three-dimensional branching tree-like structures, to deliver light to these tissues. Also, quantum dots may be conjugated with channel proteins introduced via a vector. In embodiments of a polymeric fiber optic material, the particles and/or nanowires may be included in at least a surface layer of the polymer, with or without conjugated biomolecules with either direct or indirect linkage and/or non-conjugated biomolecules. In one embodiment an implanted three-dimensional branching fiber optic structure coated with membrane ion channel activators is provided, e.g., implanted, and is activated with light to stimulate an organ such as the brain in multiple separate areas simultaneously. In one embodiment the structure is positioned on the organ surface. In one embodiment the structure is positioned internally in the organ. In one embodiment an implanted tubular structure is provided to bridge or to surround cut nerves. In one embodiment such a structure is coated with appropriate stimulating compounds, e.g., nerve growth factor (e.g., Brimodin, etc.) to stimulate axonal growth, or is coated with appropriate inhibiting compounds to inhibit scar formation at the site of trauma. In one embodiment such a structure is provided with stimulating or inhibiting compounds administered separately. In one embodiment the structures may be positioned on and/or in any organ or system, e.g., spinal cord, peripheral nerves, heart, brain, etc.

In one embodiment, the glial cells, because of their mobility, participate in various immune responses or inflammatory responses of the body and because neuronal degeneration is induced by an external source, such as viruses etc., or internal genetic mutation is associated with release of metalloproteinases and other enzymes, that affect all cells even normal tissue cells, therefore the addition of anti-inflammatory compounds, such as Rock inhibitors, e.g., Fasudil (HA-1077), a selective RhoA/Rho kinase (ROCK) inhibitor, or Y-27632, small molecule inhibitor of ROCK1 and ROCK2, Simvastatin along with modification of the genetic mutation or pathologic processes go hand in hand.

The inventive method includes mechanisms to delay, minimize, reduce, alleviate, correct, or prevent electro-sensory polarization pathologies and inflammatory processes that aggravate the neuronal degeneration in retina, such as in diabetic retinopathy, or in the brain of the diabetic patient or Alzheimer's disease using simultaneously anti-inflammatory agents, such as Rock inhibitors, e.g., fasudil, Simvastatin, etc., NSAIDs, aspirin, mmp inhibitors, such as doxycycline, etc., along with gene therapy or controlling the cell membrane polarization with QDs or nanoparticles. Such mechanisms may attenuate cellular damage resulting from abnormal polarization, reduced polarization, enhanced polarization, hyperpolarization, or loss of polarization. These polarization defects may be of any type and/or cell combination, and may stimulate and/or de-stimulate the cell(s). They may, for example, be transient in one cell type, sustained in one cell type, propagated to affect adjacent cells, propagated along a network to affect non-adjacent cells, etc.

Attaching functionalized nanocrystal quantum dots conjugated with Rock inhibitors or semiconductor layers increases the photovoltaic efficiencies. The semiconductor solar cells work by using the energy of incoming photons to raise electrons from the semiconductor's valence band to its conduction band. A potential barrier formed at the junction between p-type and n-type regions of the semiconductor forces the pairs to split, thereby producing a current, thus influencing, changing, or regulating the polarization of a membrane. The particles are stimulated by using an external or internal energy source. Polarization of the particles is regulated by producing or varying the current. The particles are used to stimulate the cell membrane by varying the input energy from the energy source.

One embodiment provides nano- or micro-sized solar cells to regulate the polarity of excitable cells. As previously described, excitable cells include, but are not limited to, sensory cells such as the retina of the eye, all three types of muscle cells, and central and peripheral system nerve cells. Such nano- or micro-sized solar cells are hereinafter generally referred to as particles 125 as shown in FIG. 8. In one embodiment, particles encompass any and all sizes which permit passage through intercellular and/or intracellular spaces in the organ or area of the organ of interest. For example, intercellular spaces in the retina are about 30 angstroms (30×10-8), so that particles for intercellular retinal distribution may be sized for these spaces, as known to one skilled in the art. In one embodiment, the particles are inserted within the lipid bilayer of liposomes and, following administration, the particles become incorporated within the cell membrane of a desired cell type or types.

The solar cell nano- and/or micro-particles 125 are three dimensional semiconductor devices. The particles use light energy or ultrasound energy to generate electrical energy to provide a photovoltaic effect. In one embodiment, the particle material is a ceramic. In another embodiment, the particle material is a plastic. In another embodiment, the particle material is silicon. Particles of crystalline silicon may be monocrystalline cells, poly or multicrystalline cells, or ribbon silicon having a multicrystalline structure. These are fabricated as microscale or nanoscale particles that are administered to a patient.

The particles may be a nanocrystal of synthetic silicon, gallium/arsenide, cadmium/selenium, copper/indium/gallium/selenide, zinc sulfide, indium/gallium/phosphide, gallium arsenide, indium/gallium nitride, and are synthesized controlling crystal conformations and sizes. In one embodiment, the nanoparticle may comprise a nanocrystal, such as cadmium/selenium (Cd/Se), and a metal. For example, a CdSe/Au nanometer-sized composite particle may be synthesized through a two-step procedure, where CdSe nanorods are formed by the reaction of Cd and Se precursors in a mixture of trioctylphosphine oxide and an alkylphosphonic acid to form rod-shaped CdSe nanoparticles, and the CdSe rods are treated with a mixture of gold chloride, didodecyldimethyl-ammonium bromide, and hexadecylamine to stabilize the nanocrystals and to reduce the gold chloride to elemental gold. Because the two ends of the CdSe rods differ crystallographically, and therefore chemically, control of growth conditions allows growth of Au particles preferentially on one end of each rod. In addition to CdSe/Au particles, one skilled in the art will readily recognize that particles can be constructed from a variety of other semiconductor/metal and semiconductor/semiconductor hetero-junctions. For example, particles based upon semiconductor/metal hetero-junctions between group II-VI, IV, III-V, IV-VI, referring to groups of the periodic table, metal-oxide, or organic semiconductors and a metal, and in particular those based upon Si/Au, GaAs/Au, InAs/Au, and PbS/Au hetero-junctions, can be used in the same way as those discussed here.

The particles (quantum dots and/or semiconductor nanowires) may also be biocompatible short peptides made of naturally occurring amino acids that have the optical and electronic properties of semiconductor nano-crystals. One example is short peptides of phenylalanine. The particles can consist of both inorganic or organic materials, as previously described.

The particles may be coated with biocompatible mono- or bilayers of phospholipid a protein, a peptide polyethylene glycol (PEG) that can be used as a scaffold to aid in biocompatibility of the particle. The particles can be entirely or partially biodegradable.

The particles may also be included in or coated on a bioabsorbable or non-bioabsorbable but biocompatible polymer structured or configured as a fiber, a tube, a substantially two-dimensional structure, or a three-dimensional structure to fit any anatomical or physioloical site. The coated polymer structure may be any desirable length or size in order to maintain its position with respect to a tissue structure. The therapeutic stimulation of the polymer and adjacent tissue may stimulate and/or inhibit the excitation of cells depending upon the wavelength of the applied light and the character of the one or more types of particles associated with it, with differing parts of the polymer, e.g., the front and back sides of a substantially two-dimensional structure, having different particles in order to have different effects upon the target cells adjoining those parts.

In one embodiment, the particles are delivered to the retinal cell cytoplasm or nucleus or cell membrane, regardless of the particular injection site in the eye. In one embodiment, the particles are introduced into the central nervous system, e.g., by injection. In one embodiment, the quantum dots are covalently linked, i.e., conjugated, with natural or synthetic biomolecules (e.g., proteins, peptides, nucleic acids, oligonucleotides, etc.) that introduce a non-viral vector or viral vectors (e.g., adeno-associated virus (AAV) for retinal gene therapy. Such a vector and/or the bound quantum dots/semiconductor nanowires can be labeled for visualization, tracking, sensing, etc. For example, the quantum dots can be labeled or tagged with a signal recognition moiety. Such a vector can incorporate quantum dots into the viral capsid using, e.g., (poly)ethylene glycol (PEG) moieties. Combinations of these embodiments are contemplated and included in the inventive method, using methods known by one skilled in the art and as subsequently described.

In one embodiment, the particles are conjugated with a moiety such as an ocular peptide or protein, to result in a biologically active quantum dot conjugate. Such conjugation allows the therapeutic effect to be controlled and specific, while sensing and tracking the conjugate location, function, etc. in, e.g., the retina.

Examples of such ocular peptides and proteins include, but are not limited to, membrane-bound G-protein coupled photoreceptors (opsins, including the rod cell night vision pigment rhodopsin and cone cell color vision proteins), and members of the family of ocular transport proteins (aquaporins).

In one embodiment, short peptides of naturally occurring amino acids that have the optical and electronic properties of semiconductor nano-crystals are conjugated to the particles. One non-limiting example of such a short peptide is (poly)phenylalanine. In these embodiments, the resulting conjugate contains both inorganic and organic materials, as previously described. In one embodiment, the conjugates may be coated with biocompatible mono- or bilayers of phospholipid, protein, and/or a (poly)ethylene glycol (PEG) molecule that can be used as a scaffold to aid in biocompatibility of the particle. Any of these organic moieties may be utilized to ionically, electronically or covalently form the biologically active conjugates. The conjugates are entirely or partially biodegradable.

In one embodiment, a particle conjugated to a vector is capable of modifying an ocular gene, e.g., a gene of a retinal cell. In this embodiment, the quantum dot and/or semiconductor nanowire, besides regulating membrane polarity of an excitable cell such as a retinal cell, also provides therapy to ameliorate or prevent a genetically based retinal disease (e.g., retinitis pigmentosa). In one embodiment, the vector may be a plasmid vector, a binary vector, a cloning vector, an expression vector, a shuttle vector, or a viral vector as known to one skilled in the art. The vector typically contains a promoter, a means for replicating the vector, a coding region, and an efficiency increasing region. In one embodiment, the vector is a virus such as an adenovirus, an adeno-associated virus (AAV), a retrovirus, and other viral vectors for gene therapy, as known to one skilled in the art. As one non-limiting example, particles are functionalized and/or linked to viral vectors using (poly)ethylene glycol (PEG) moieties. The number of PEGS can be varied depending on, e.g., ocular site, need to enhanced hydrophilicity, protein size, etc. The viral vector and particle are combined in the presence of at least one biocompatible adjuvant, suspension agent, surfactant, etc. Particles may be coated with or linked to, e.g., folate, polydopamine, etc. so that these molecules are targeted intracellularly, extracellularly, to a cell membrane, to a specific cellular site or organelle, etc.

Conjugation of quantum dots to viral capsids permits in vivo observation of retinal neurons and the individual glycine receptors in living neurons. A single quantum dot can be recognized by optical coherence tomography (OCT) and can be counted, tracked, assessed, monitored, and evaluated for longevity and efficacy, and hence therapy can also be monitored, over time.

In one embodiment, particles associated with other biomolecules, e.g., conjugated with halorrhodopson, conjugated with customized gold nanoparticles, are used to regulate, i.e., stimulate or inhibit, action potential of a neuron. Quantum dots and semiconductor nanowires can be associated with, e.g., conjugated with, a virus, a virus capsid, a cell penetrating protein, and/or other molecule(s) to stimulate specific neurons or specific neuronal function, or may be provided with appropriate stem cells. In one embodiment, these combinations may stimulate or inhibit the action potential of cells depending upon the wavelength(s) of light applied to them to provide a highly selective “on or off” form of external regulation.

In one embodiment, covalent conjugation may not be required or desired, and in this embodiment particles may be simply associated with a viral vector. In one embodiment, quantum dots may be mixed with an appropriate viral vector in the presence of a cationic polymer, e.g. hexadimethrine bromide POLYBRENE® to form a colloidal complex suitable for introducing into a retinal cell. In one embodiment, particles are tagged with an amide, a thiol, etc. using electrostatic interaction along with functionalizing means known to one skilled in the art.

In one embodiment, the quantum dots that are conjugated or associated with a biomolecule are delivered to a target cell cytoplasm or nucleus, using described methods and/or methods known in the art. In one embodiment, the biomolecule comprises nucleic acid, such as DNA and RNA, as well as synthetic congeners thereof. Non-limiting examples of nucleic acids may include plasmid DNA encoding protein or inhibitory RNA producing nucleotide sequences, synthetic sequences of single or double strands, missense, antisense, nonsense, on and off and rate regulatory nucleotides that control protein, peptide, and nucleic acid production. Nucleic acids include, but are not limited to, genomic DNA, cDNA, RNAi, siRNA, shRNA, mRNA, tRNA, rRNA, microRNA, hybrid sequences or synthetic or semi-synthetic sequences. Each of these may be naturally occuring or synthetic. Each of these may be of human, plant, bacterial, yeast, viral, etc. origin. Each of these may be any size, e.g., ranging from oligonucleotides to chromosomes. They may be obtained by any technique known to one skilled in the art.

In one embodiment, a nucleotide sequence may also encode products for synthesis or inhibition of a therapeutic protein such as, but not limited to, anti-cancer agents, growth factors, hypoglycemic agents, anti-angiogenic agents, bacterial antigens, viral antigens, tumor antigens, and/or metabolic enzymes. Examples of anti-cancer agents include, but are not limited to, interleukin-2, interleukin-4, interleukin-7, interleukin-12, interleukin-15, interferon-α, interferon-βinterferon-γ, colony stimulating factor, granulocyte-macrophage stimulating factor, anti-angiogenic agents, tumor suppressor genes, thymidine kinase, eNOS, iNOS, p53, p16, TNF-α, Fas-ligand, mutated oncogenes, tumor antigens, viral antigens, and/or bacterial antigens. In one embodiment, plasmid DNA may encode for an RNAi molecule designed to inhibit protein(s) involved in tumor or other hyperproliferative cells' growth or maintenance. In one embodiment, a plasmid DNA may simultaneously encode a therapeutic protein and one or more RNAi molecules. In one embodiment, a nucleic acid may be a mixture of plasmid DNA and synthetic RNA, including sense RNA, antisense RNA, ribozymes, etc.

In one embodiment, the disclosed quantum dot-nucleic acid complex is administered to an individual, e.g., patient in need of such therapy, to ameliorate a genetic disease. In one, embodiment, the disclosed quantum dot-nucleic acid complex is administered to an individual, e.g., a patient with a tumor, to reduce the tumor burden, ameliorate tumor effects, treat the tumor, etc. Therapy may be curative, palliative, remediation, etc. and may be either total or partial, and may be either therapeutic or preventive. The disclosed quantum dot-nucleic acid complex may be used in gene targeting or knockout of specific genes, for e.g., with at least one engineered nuclease, tumor suppressor gene(s), etc. In one embodiment, the disclosed quantum dot-nucleic acid complex contains a wild-type or non-mutated form of a gene or part of a gene, and is introduced into a cell or cells, with the wild-type or non-mutated form of the nucleic acid replacing a defective and/or mutated form of the nucleic acid, e.g., DNA. Because the nucleic acid may be synthetic oligonucleotide, the disclosed gene therapy can replace missing or defective copies of a nucleic acid, and/or restore or impart a new function to overcome a disease.

In one embodiment, the disclosed method of gene therapy is somatic gene therapy and thus applied to the patient undergoing therapy. In one embodiment, the disclosed method of gene therapy is germ line gene therapy and thus not limited to the patient undergoing therapy, being capable of transmission to offspring of the patient. In one embodiment, the disclosed gene therapy methods comprise delivery of a single gene or multiple genes. Multiple genes may be in a single quantum dot complex, or may be in multiple quantum dot complexes. Multiple quantum dot-nucleic acid complexes may be administered either at the same times or at different times. In embodiments where the nucleic acid in the quantum dot complex is in a linear form, e.g., a linear DNA fragment, when introduced into cells, the linear nucleic acid molecules are ligated end-to-end by intracellular enzymes to form long tandem arrays, which promote integration of the nucleic acid into a chromosome.

In embodiments, the disclosed gene therapy methods can be provided alone, or in combination with additional treatments such as stem cell therapy. In one embodiment, a method for treating retinal, CNS, and cardiovascular diseases is provided by providing the disclosed quantum dot-nucleic acid complexes to the patient to effect gene therapy, along with stem cell therapy as known in the art. The therapies may be provided together or separately. In one embodiment, the disclosed method may be provided as part of a combination therapy additionally comprising, e.g., agents such as immunomodulators, anti-VEGF agents, anti-integrins, anti-inflammatory agents, antibiotics, anti-viral agents, anti-fungal agents, anti-proliferative agents, anti-cancer agents, etc.

In one embodiment, a rho-associated protein kinase (ROCK) inhibitor is administered to a patient to reduce an inflammatory process and facilitate nerve growth in an ocular or neurodegenerative disorder such as Alzheimer's disease, or retinal degeneration, with the ROCK inhibitor in a mucophilic preparation, gold nanoparticles, or microparticles that contain one or more of cell penetrating peptide (CPP), activated CPP (ACPP), cyclic CPP, chitosan, dendrimer, (poly)glycolic acid (PGA), (poly)lactic acid (PLA), (poly)glycolic (poly)lactic acid) (PGLA), antibody, growth factor, and/or opsin gene.

In one embodiment, a plurality of pluripotent cells, e.g., modified human skin stem cells, cultured stem cells, genetically modified stem cells, embryonic stem cells, mesenchymal stem cells, neuronal stem cells, glial stem cells, and/or stem cells having complement receptor 35, etc., and a gene therapy method, e.g., a programmable gene editing nuclease such as meganuclease, zinc-finger nuclease (ZFN), CRISPR Cas 9 system, transcription activator-like effector nuclease (TALENS), non-homology gene editing, presenilin 1 (PS1) and presenilin 2 (PS2) gene correction mechanisms, in Alzheimer's disease etc. can be administered with the ROCK inhibitor. In one embodiment administration as the disease process indicates may be intravenously, systemically, intravitreally, through the choroid, in the cerebrospinal fluid (CSF), topically, though the conjunctival mucosa, through the nasal mucosa, through the cornea, though the retinal optic nerve, through the nasal mucosa olfactory nerve, and the nanoparticle complex is stimulated through the skin over the nose base using a laser light, ultrasound in the brain, and/or in the spinal cord through the skin located over the site of injury.

In one embodiment, cultured glial cells sensitized to an anti-amyloid antibody (e.g., aducanumab), anti-Tau antibody, anti-entangled Tau toxic protein, and/or glycogen synthesis kinase 3 (GSK-3) inhibitor may be administered with gold nanoparticles. Cellular immunotherapy, vaccination, and/or an agent to enhance cellular proliferation may also be administered by polarization and depolarization of the glial cells microglial cells, etc.

In one embodiment, the nanoparticles coated with PEG and Rock inhibitors are administered simultaneously or sequentially with administration of a steroid, non-steroidal anti-inflammatory drug (NSAID), methylene blue, vitamin E, vitamin B complex, Nispan, and/or derivatives of methylene blue in inflammatory diseases or after traumatic injuries.

In embodiments using the CRISPR/Cas9 system, an inhibitor of the CRISPR Cas system may be provided to correct or halt gene editing.

In one embodiment, the disclosed quantum dot-nucleic acid complex may be targeted and/or directed to a specific region of the body, e.g., a specific organ, tissue type, and/or cell type, where the targeted location may be the site of a disease or a location affected by a disease. In one embodiment, the quantum dot contains or is provided with a coating to enhance or impart biocompatibility and/or cell selectivity using, e.g., an antibody, receptor, etc. that directs the complex to a desired location, e.g., a tumor site, a specific receptor, etc. In one embodiment, targeting or directing the complex may occur using a selected site to provide access to the desired location. For example, in ocular diseases, the disclosed quantum dot-nucleic acid complex may be injected intravitreally, introduced into the cornea, choroid, retina, etc., provided as a topical formulation, etc., as also described herein.

In one embodiment, the disclosed quantum dot-nucleic acid complex may provide both therapeutic and imaging functions. For example, to evaluate the effect of gene modification in the eye, visual acuity electroretinogram, visual field, OCT, ophthalmoscopy, and/or photography may be employed. In one embodiment, the disclosed quantum dot-nucleic acid complex is imaged by photography and/or optical coherence tomography (OCT) in accessible regions such as the eye and skin, and/or is imaged by magnetic resonance imaging (MRI). The ability to image the disclosed complex, particularly when the complex comprises a targeting moiety, provides diagnostic value. Complex accumulation, concentration, or localization at a specific site or area of the body, e.g., breast, brain, prostate, etc., is indicative that this area exhibits the disease or condition to be treated. The disclosed complex may also be imaged by more routine methods such as microscopy visualizing the complex in samples of tissue, including biopsy tissue samples, or body fluids including but not limited to blood.

In one embodiment, the disclosed quantum dot-nucleic acid complex further contains at least one moiety that binds to a tumor-specific protein marker. In one embodiment, the complex still further contains a reporter molecule, in addition to the binding moiety, e.g., an antibody directed to a tumor marker. Reporter molecules are known in the art and include, but are not limited to, molecules that are fluorescent, luminescent, phosphorescent, etc. In this embodiment the complex is administered systemically to a patient to diagnose a tumor by locating and/or imaging the protein-nucleic acid-tumor binding moiety at a tumor site. For example, following administration of the complex to a patient, a blood sample is obtained from the patient and subjected to an immunofluorescence assay and/or examined by fluorescent microscopy to detect and/or measure the amount of the tumor marker in the sample. In one embodiment, the quantum dot of the complex and the reporter molecule, such as a fluorescent dye, of the anti-tumor marker antibody have excitation (ex) and emission (em) maxima at different wavelengths, and the sample is examined at multiple wavelengths. The determination of binding by both the complex and anti-tumor marker antibody in the assay provides a more definitive determination that the tumor marker, and thus the tumor, is present in the patient. In one embodiment, the reporter molecule of the anti-tumor marker antibody has emission maxima at the green wavelengths of light. In one embodiment, the specificity of the complex for a tumor is increased by associating multiple tumor marker-binding proteins to the complex. This embodiment provides enhanced opportunities for early detection of a tumor, and prior to tumor metastasis.

In one or more embodiments, functionalized nanoparticles, such as gold, graphene, etc. are used as vectors for gene modification using non-homologous end joining or homology directed repair (HDR), where the nanoparticles are coated with a cell penetrating agent conjugated with donor DNA and a CRISPR/cas9 guide RNA, and a thermal energy source, such as light or ultrasound, heats up the nanoparticles and enhances the cell penetration and enhanced endosomal escape and enhance tissue repair.

The following disclosure demonstrates use in various therapies. In one embodiment, a method for inducing a mammalian cell to produce a recombinant protein is provided. In this embodiment, the disclosed quantum dot-nucleic acid and CRISPR/cas9 encoding the recombinant protein is provided to a patient. In one embodiment, a method for anemia therapy in a patient is provided. In this embodiment, the disclosed quantum dot-nucleic acid encoding an angiogenic agent, e.g., erythropoietin and CRISPR/cas9, is provided to a patient, thereby providing therapy for anemia in the patient. In one embodiment, a method for vasospasm therapy in a patient is provided. In this embodiment, the disclosed quantum dot-nucleic acid encoding inducible nitric oxide synthase (iNOS) and CRISPR/cas9 is provided to a patient, thereby providing therapy for vasospasm in the patient. In one embodiment, a method for vasospasm therapy in a patient is provided. In this embodiment, the disclosed quantum dot-nucleic acid is conjugated with a Rock inhibitor and encoding inducible nitric oxide synthase (iNOS) and CRISPR/cas9 is provided to a patient, thereby providing therapy for vasospasm in the patient. In one embodiment, a method for improving cell survival in a patient is provided. In this embodiment, the disclosed quantum dot-nucleic acid encoding a heat shock protein and CRISPR/Cas9 is provided to a patient, therapy providing therapy for increased cell survival. In one embodiment, a method for decreasing incidence of a restenosis of a blood vessel, following a procedure that enlarges the blood vessel, is provided. In this embodiment, the disclosed quantum dot-nucleic acid encoding a heat shock protein and CRISPR/cas9 is provided to a patient, thereby decreasing incidence of a restenosis in the patient. In one embodiment, a method for increasing growth from a hair follicle in a scalp of a patient is provided. In this embodiment, the disclosed quantum dot-nucleic acid encoding a telomerase and CRISPR/cas9 or an immunosuppressive protein is provided to a patient, thereby increasing hair growth from a hair follicle. In one embodiment, a method of inducing expression of an enzyme with antioxidant activity in a cell is provided. In this embodiment, the quantum dot-nucleic acid encoding the enzyme and CRISPR/cas9 is provided to a patient, thereby inducing expression of the enzyme with antioxidant activity in a cell. In one embodiment, a method of cystic fibrosis therapy is provided. In this embodiment, the disclosed quantum dot-nucleic acid conjugated with a Rock inhibitor and encoding Cystic Fibrosis Transmembrane Conductance Regulator (CFTR) and CRISPR/cas9 is provided, thereby providing therapy for cystic fibrosis in the patient. In this embodiment, the disclosed quantum dot-nucleic acid and CRISPR/cas9 conjugated with Rock inhibitors and encoding a Cystic Fibrosis Transmembrane Conductance Regulator (CFTR) is provided, thereby providing therapy for cystic fibrosis in the patient. In one embodiment, a method for treating an X-linked agammaglobulinemia in a patient is provided. In this embodiment, the disclosed quantum dot-nucleic acid and CRISPR/cas9 encoding a Bruton's tyrosine kinase and CRISPR/cas9 is provided to a patient, thereby providing therapy for an X-linked agammaglobulinemia in the patient. In one embodiment, a method of therapy for an adenosine deaminase severe combined immunodeficiency (ADA SCID) in a patient is provided. In this embodiment, the disclosed quantum dot-nucleic acid and CRISPR/cas9 complex encoding an ADA is provided to a patient, thereby providing ADA SCID therapy in the patient. In one embodiment, a method of therapy for hemophilia B in a patient is provided. In this embodiment, the disclosed quantum dot-nucleic acid encoding Factor IX is provided to a patient, thereby providing therapy for hemophilia B. In one embodiment, a method of therapy for spinal muscular atrophy in a patient is provided. In this embodiment, the disclosed quantum dot-nucleic acid complex encoding SMN-1 and CRISPR/cas9 is provided to a patient, thereby providing therapy for spinal muscular atrophy in the patient. In one embodiment, a method for providing therapy for exudative age related macular degeneration (AMD) in a patient is provided. In this embodiment, the disclosed quantum dot-nucleic acid and CRISPR/cas9 encoding an anti-VEGF protein is provided, thereby providing therapy to the patient with exudative AMD. In one embodiment, the anti-VEGF protein is sFlt-1, which is a naturally occurring protein antagonist of VEGF. In one embodiment, a method of therapy for choriodemia in a patient is provided. In this embodiment, the quantum dot-nucleic acid complex encoding Rab-Escort Protein (REP-1) and CRISPR/cas9 is provided to the patient, thereby providing therapy for choriodemia in the patient. In one embodiment, a method of therapy for Leber's congenital amaurosis (LCA) in a patient is provided. In this embodiment, the quantum dot-nucleic acid encoding RPE65 and CRISPR/cas9 is provided to the patient, thereby providing therapy for Leber's congenital amaurosis in the patient. RPE65 is an RPE-specific 65-kDA protein involved in conversion of all-trans retinol to 11-cis retinal during phototransduction, and has been implicated as a genetic defect in LCA.

In one embodiment, a method of therapy for retinitis pigmentosa in a patient is provided. In this embodiment, the quantum dot-nucleic acid encoding MERTK and CRISPR/cas9 is provided to the patient, thereby providing therapy for retinitis pigmentosa in the patient. In one embodiment, a method of therapy for Stargardt's syndrome in a patient is provided. In this embodiment, quantum dot-nucleic acid encoding ABC4 and CRISPR/cas9 is provided to a patient, thereby providing therapy for Stargardt's syndrome in the patient. The ABCA4 gene produces a protein involved in energy transport to and from photoreceptor cells in the retina. In one embodiment, a method of therapy for Usher's syndrome (1B) in a patient is provided. In this embodiment, the quantum dot-nucleic acid encoding MY07A and CRISPR/cas9 is provided to a patient, thereby providing therapy for Usher's syndrome (1B) in the patient.

In one embodiment, a method of therapy for advanced and/or metastatic pancreatic cancer in a patient is provided. In this embodiment, the quantum dot-nucleic acid encoding two genes, somatostatin receptor subtype 2 (sst2) and deoxycitidine kinase::uridylmonophosphate kinase (dck::umk), which exhibit complementary therapeutic effects and CRISPR/cas9, is provided to the patient, thereby providing therapy for advanced and/or metastatic pancreatic cancer in the patient. Both genes induce an antitumor bystander effect and render gemcitabine treatment more efficient.

In general, in any embodiment, the disclosed quantum dot-nucleic acid complex encoding a gene can be replaced with a gold nanoparticle DNA encoding the desired gene and CRISPR/cas9 administered to a patient, thereby providing therapy in the patient from a gene mutation, etc., such as in Alzheimer's disease or diabetes, e.g., associated with loss of telomeres.

In one embodiment, the quantum dots or gold nanoparticles, which are conjugated with a Rock inhibitor and nucleic acid encoding a telomerase and CRISPR/cas9, is administered to the patient to treat inflammatory processes affecting the brain tissue, e.g., in Alzheimer's disease, or the retina, e.g., in diabetes with diabetic retinopathy.

In embodiments, other ocular pathological conditions as well as additional therapeutic nucleic acids and CRISPR/cas9 may be provided, some of which were previously described. Examples include, but are not limited to, retinitis pigmentosa, color blindness, wet and dry ARMD, diabetic retinopathy, corneal dystrophies, Meesman syndrome, Alzheimer's disease, Fuchs syndrome, granular and macular corneal dystrophies, keratoconous, Sejorgen's syndrome, inherited glaucoma, retinohyaloidopathies, congenital cataract, Marfan syndrome, choridermia x-linked retinoschisis, achromatopsia, etc.

In one alternative embodiment, one may use a peptide nucleic acid (PNA) to replace CRISPR/cas9 in any described embodiments in this application to avoid misfiring of the enzyme CRISPR/cas9. Peptide nucleic acid (PNA) is a synthesized polymer that triggers the cell to engage the cell's DNA to correct the mutated gene sequences. PNAs combine a synthetic protein polyamide backbone with specific genomic target site or nucleobases present in DNA and RNA creating PNA/DNA/PNA complex. The PNA molecules are paired with a donor DNA encoding the corrected gene sequence, such as opsin or another gene and conjugated with antibody coated gold nanoparticles coated with poly(lactic-co-glycolic acid) (PLGA) and cell penetrating peptides or activatable CPPs and Rock inhibitors to enhance cell penetration and escape from the endosomes and administered either topically to the eye or nasal mucosa, intravitreally, in the cerebrospinal fluid, etc. for treatment of retinal or brain neurodegenerative diseases such as Alzheimer's disease, Parkinson disease, or degenerative disease of the retina, such as retinitis pigmentosa, LCA, diabetic retinopathy.

In one embodiment, a method for anemia therapy in a patient is provided. In this embodiment, the disclosed quantum dot, or gold nanoparticles-nucleic acid encoding an angiogenic agent, e.g., erythropoietin and creating PNA/DNA/PNA complex, is provided to a patient, thereby providing therapy for anemia in the patient. In one embodiment, a method for vasospasm therapy in a patient is provided. In this embodiment, the disclosed quantum dot or gold nanoparticle coated with poly(lactic-co-glycolic acid) (PLGA) and cell penetrating peptides or activatable CPPs and Rock inhibitors-nucleic acid encoding inducible nitric oxide synthase (iNOS) 1 creating PNA/DNA/PNA complex is provided to a patient, thereby providing therapy for vasospasm in the patient. In one embodiment, a method for vasospasm therapy in a patient is provided. In this embodiment, the disclosed quantum dot-nucleic acid or gold nanoparticle coated with poly(lactic-co-glycolic acid) (PLGA) and cell penetrating peptides or activatable CPPs and Rock inhibitors-nucleic acid conjugated with Rock inhibitora and encoding inducible nitric oxide synthase (iNOS) creating a PNA/DNA/PNA complex is provided to a patient, thereby providing therapy for vasospasm in the patient. In one embodiment, a method for improving cell survival in a patient is provided. In this embodiment, the disclosed quantum dot-nucleic acid or gold nanoparticle coated with poly(lactic-co-glycolic acid) (PLGA) and cell penetrating peptides or activatable CPPs and Rock inhibitors-nucleic acid encoding a heat shock protein creating PNA/DNA/PNA complex is provided to a patient, providing therapy for increased cell survival. In one embodiment, a method for decreasing incidence of a restenosis of a blood vessel, following a procedure that enlarges the blood vessel, is provided. In this embodiment, the disclosed quantum dot-nucleic acid or gold nanoparticle coated with poly(lactic-co-glycolic acid) (PLGA) and cell penetrating peptides or activatable CPPs and Rock inhibitors-nucleic acid encoding a heat shock protein and creating PNA/DNA/PNA complex is provided to a patient, thereby decreasing incidence of a restenosis in the patient. In one embodiment, a method for increasing growth from a hair follicle in a scalp of a patient is provided. In this embodiment, the disclosed quantum dot-nucleic acid or gold nanoparticle coated with poly(lactic-co-glycolic acid) (PLGA) and cell penetrating peptides or activatable CPPs and Rock inhibitors-nucleic acid encoding a telomerase and creating a PNA/DNA/PNA complex or an immunosuppressive protein is provided to a patient, thereby increasing hair growth from a hair follicle. In one embodiment, a method of inducing expression of an enzyme with antioxidant activity in a cell is provided. In this embodiment, the quantum dot-nucleic acid or gold nanoparticle coated with poly(lactic-co-glycolic acid) (PLGA) and cell penetrating peptides or activatable CPPs and Rock inhibitors-nucleic acid encoding the enzyme and creating PNA/DNA/PNA complex is provided to a patient, thereby inducing expression of the enzyme with antioxidant activity in a cell. In one embodiment, a method of cystic fibrosis therapy is provided. In this embodiment, the disclosed quantum dot-nucleic acid or gold nanoparticle coated with poly(lactic-co-glycolic acid) (PLGA) and cell penetrating peptides or activatable CPPs and a nucleic acid conjugated with a Rock inhibitor and encoding Cystic Fibrosis Transmembrane Conductance Regulator (CFTR) and creating PNA/DNA/PNA complex is provided, thereby providing therapy for cystic fibrosis in the patient. In this embodiment, the disclosed quantum dot-nucleic acid or gold nanoparticle coated with poly(lactic-co-glycolic acid) (PLGA) and cell penetrating peptides or activatable CPPs and Rock inhibitors-nucleic acid and creating PNA/DNA/PNA complex conjugated with Rock inhibitors and encoding Cystic Fibrosis Transmembrane Conductance Regulator (CFTR) is provided, thereby providing therapy for cystic fibrosis in the patient. In one embodiment, a method for treating an X-linked agammaglobulinemia in a patient is provided. In this embodiment, the disclosed quantum dot-nucleic acid or gold nanoparticle coated with poly(lactic-co-glycolic acid) (PLGA) and cell penetrating peptides or activatable CPPs and Rock inhibitors-nucleic acid and creating PNA/DNA/PNA complex encoding a Bruton's tyrosine kinase and is provided to a patient, thereby providing therapy for an X-linked agammaglobulinemia in the patient. In one embodiment, a method of therapy for an adenosine deaminase severe combined immunodeficiency (ADA SCID) in a patient is provided. In this embodiment, the disclosed quantum dot-nucleic acid or gold nanoparticle coated with poly(lactic-co-glycolic acid) (PLGA) and cell penetrating peptides or activatable CPPs and Rock inhibitors-nucleic acid and creating PNA/DNA/PNA complex encoding an ADA is provided to a patient, thereby providing ADA SCID therapy in the patient. In one embodiment, a method of therapy for hemophilia B in a patient is provided. In this embodiment, the disclosed quantum dot-nucleic acid or gold nanoparticle coated with poly(lactic-co-glycolic acid) (PLGA) and cell penetrating peptides or activatable CPPs and Rock inhibitors-nucleic acid encoding Factor IX is provided to a patient, thereby providing therapy for hemophilia B. In one embodiment, a method of therapy for spinal muscular atrophy in a patient is provided. In this embodiment, the disclosed quantum dot-nucleic acid complex encoding SMN-1 and creating PNA/DNA/PNA complex or gold nanoparticle coated with poly(lactic-co-glycolic acid) (PLGA) and cell penetrating peptides or activatable CPPs and Rock inhibitors-nucleic acid encoding SMN-1 is provided to a patient, thereby providing therapy for spinal muscular atrophy in the patient. In one embodiment, a method for providing therapy for exudative age related macular degeneration (AMD) in a patient is provided. In this embodiment, the disclosed quantum dot-nucleic acid and/or gold nanoparticle coated with poly(lactic-co-glycolic acid) (PLGA) and cell penetrating peptides or activatable CPPs and Rock inhibitors-nucleic acid creating a PNA/DNA/PNA complex encoding an anti-VEGF protein is provided, thereby providing therapy to the patient with exudative AMD. In one embodiment, the anti-VEGF protein is sFlt-1, which is a naturally occurring protein antagonist of VEGF. In one embodiment, a method of therapy for choriodemia in a patient is provided. In this embodiment, the quantum dot-nucleic acid complex encoding Rab-Escort Protein (REP-1) and creating PNA/DNA/PNA complex or gold nanoparticle coated with poly(lactic-co-glycolic acid) (PLGA) and cell penetrating peptides or activatable CPPs and Rock inhibitors-nucleic acid complex encoding Rab-Escort Protein (REP-1) is provided to the patient, thereby providing therapy for choriodemia in the patient. In one embodiment, a method of therapy for Leber's congenital amaurosis (LCA) in a patient is provided. In this embodiment, the quantum dot-nucleic acid or gold nanoparticle coated with poly(lactic-co-glycolic acid) (PLGA) and cell penetrating peptides or activatable CPPs and Rock inhibitors-nucleic acid creating PNA/DNA/PNA complex encoding RPE65 and creating PNA/DNA/PNA complex is provided to the patient, thereby providing therapy for Leber's congenital amaurosis in the patient. RPE65 is an RPE-specific 65-kDA protein involved in conversion of all-trans retinol to 11-cis retinal during phototransduction, and has been implicated as a genetic defect in LCA.

In one embodiment, a method of therapy for retinitis pigmentosa in a patient is provided. In this embodiment, the quantum dot-nucleic acid or gold nanoparticle coated with poly(lactic-co-glycolic acid) (PLGA) and cell penetrating peptides or activatable CPPs and Rock inhibitors-nucleic acid encoding MERTK and creating a PNA/DNA/PNA complex is provided to the patient, thereby providing therapy for retinitis pigmentosa in the patient. In one embodiment, a method of therapy for Stargardt's syndrome in a patient is provided. In this embodiment, quantum dot-nucleic acid or gold nanoparticle coated with poly(lactic-co-glycolic acid) (PLGA) and cell penetrating peptides or activatable CPPs and Rock inhibitors-nucleic acid encoding ABC4 and creating a PNA/DNA/PNA complex is provided to a patient, thereby providing therapy for Stargardt's syndrome in the patient. The ABCA4 gene produces a protein involved in energy transport to and from photoreceptor cells in the retina. In one embodiment, a method of therapy for Usher's syndrome (1B) in a patient is provided. In this embodiment, the quantum dot-nucleic acid or gold nanoparticle coated with poly(lactic-co-glycolic acid) (PLGA) and cell penetrating peptides or activatable CPPs and Rock inhibitors-nucleic acid encoding MY07A and a creating PNA/DNA/PNA complex is provided to a patient, thereby providing therapy for Usher's syndrome (1B) in the patient.

In one embodiment, a method of therapy for advanced and/or metastatic pancreatic cancer in a patient is provided. In this embodiment, the quantum dot-nucleic acid or antibody coated gold nanoparticle coated with poly(lactic-co-glycolic acid) (PLGA) and cell penetrating peptides or activatable CPPs and Rock inhibitors-nucleic acid encoding two genes and creating PNA/DNA/PNA complex, somatostatin receptor subtype 2 (sst2) and deoxycitidine kinase::uridylmonophosphate kinase (dck::umk), which exhibit complementary therapeutic effects and creating PNA/DNA/PNA complex, is provided to the patient, thereby providing therapy for advanced and/or metastatic pancreatic cancer in the patient. Both genes induce an antitumor bystander effect and render gemcitabine treatment more efficient.

In general in any embodiment, the disclosed quantum dot-nucleic acid complex or gold nanoparticle coated with poly(lactic-co-glycolic acid) (PLGA) and cell penetrating peptides or activatable CPPs and Rock inhibitors-nucleic acid creating PNA/DNA/PNA complex encoding the desired gene, and/or presenilin 1 and presenilin 2 is administered to a patient, thereby providing therapy in the patient from a gene mutation etc., such as in Alzheimer's disease or diabetes, e.g., associated with loss of telomeres.

In one embodiment, the quantum dot or gold nanoparticles conjugated with a Rock inhibitor and -nucleic acid encoding a telomerase and creating PNA/DNA/PNA complex is administered to patient to treat inflammatory processes affecting the brain tissue in Alzheimer's disease or the retina, e.g., in diabetes with diabetic retinopathy.

In embodiments, other ocular pathological conditions as well as additional therapeutic nucleic acids and creating PNA/DNA/PNA complex may be provided, some of which were previously described. Examples include, but are not limited to, retinitis pigmentosa, color blindness, wet and dry ARMD, diabetic retinopathy, corneal dystrophies, Meesman syndrome, Alzheimer's disease, Fuchs syndrome, granular and macular corneal dystrophies, keratoconous, Sejorgen's syndrome, inherited glaucoma, retinohyaloidopathies, congenital cataract, Marfan syndrome, choridermia x-linked retinoschisis, achromatopsia, etc. and additional therapeutic nucleic acids and creating a PNA/DNA/PNA complex is provided, to ameliorate the disease process.

The administration site, location, and/or method of the disclosed quantum dot-nucleic acid complex is not limited. In one embodiment, the disclosed quantum dot-nucleic acid complex may be injected into a vein or artery. In one embodiment, the disclosed quantum dot-nucleic acid complex may be introduced into the cerebrospinal fluid, ventricles, CNS, spinal cord, etc. for therapy of numerous CNS diseases such as Alzheimer's disease, Parkinson's disease, multiple sclerosis, etc. The disclosed method may be used as therapy for patients with spinal muscular dystrophy, muscular dystrophy, diseases affecting myeloid cells, chronic lymphocytic leukemia, multiple myeloma, malignant tumors, melanomas, cancers of various organs including breast, intestine, prostate, CNS, glioblastoma, sarcoma, etc. In addition, the present methods can be used to provide therapy for cystic fibrosis, hemophilia, and sickle cell disease.

In one embodiment, the disclosed quantum dot-nucleic acid complex additionally contains a magnetic or paramagnetic nanoparticle that facilitates introduction of the complex into a cell. In one embodiment, the complex comprises a quantum dot or antibody coated gold nanoparticle coated with poly(lactic-co-glycolic acid) (PLGA) and cell penetrating peptides or activatable CPPs and Rock inhibitors-nucleic acid creating PNA/DNA/PNA complex conjugated with a targeting moiety and a biomolecule, such as a gene, DNA, RNA, RNAi, sRNA, plasmid, etc., and a magnetic or paramagnetic nanoparticle also conjugated with the targeting moiety. In embodiments, the targeting moiety is an antibody or a ligand for a receptor.

In one embodiment, a method is provided for introducing the described complex into a desired cell, and thus for introducing the biomolecule, such as a gene for stimulating or silencing cell or tumor cell function. In this embodiment the complex is administered, either systemically or locally, to reach a desired cell. An energy source is then applied, e.g., an alternating magnetic field, electromagnetic radiation, etc., causing a temperature increase in the magnetic or paramagnetic nanoparticle. This temperature increase perturbs the cell membrane of the desired cell, and thus provides or enhances access to the cell at the site of the perturbation, e.g., ranging from an altered membrane conformation to a “hole”. Perturbation of the cellular membrane provides enhanced access into the cellular membrane and cytoplasm of the cell. Perturbation of the nuclear membrane provides enhanced access into the nuclear membrane and nucleoplasm of the cell.

Following access of the complex, or at least the nanoparticle containing the biomolecule, into the cell, the biomolecule provides the desired cellular effect. That is, the biomolecule is readily accessible to the cellular cytoplasm or nucleus.

In one embodiment, the quantum dot conjugated with the biomolecule may be coated with a thermosensitive polymer. Thermosensitive polymers are known in the art and include, but are not limited to, chitosan, (poly)ethylene glycol (PEG), etc. Application of an external energy source results in a slight increase in temperature, e.g., to about 39° C. to about 43° C. in one embodiment, to about 40° C. to about 42° C. in another embodiment. This slight temperature increase facilitates release of the biomolecule from the thermosensitive nanoparticles or quantum dot.

In general, the size of the quantum dot defines the wavelength of light that is absorbed by it and, similarly, the wavelength that can be emitted by it which is always longer than that absorbed. For example, a quantum dot with a size of about 200 nm-500 nm dot absorbs a longer wavelength of light than a quantum dot with a size of about 10 nm-50 nm.

As a result, the wavelength that is emitted by larger quantum dots will have a larger wavelength (carry less energy, or a shift toward the red), than those wavelengths emitted by smaller quantum dots (carry more energy, or a shift toward the blue). Therefore using different sized quantum dots, one can not only selectively stimulate the specific membrane ion channel or cells, but also make them visible differently due to their different emission of wavelengths of light.

This permits one to selectively activate cells, i.e., turn certain cells on or off, without affecting the other cells. Similarly, such cells selectively activated or turned on can also be coded with one or more different antibody, gene, biomolecule, etc. Such size tunable parameters apply equally to nanowires or nanotubes in addition to quantum dots, and can be used in addition in spectroscopy.

In one embodiment, the complex comprises quantum dots that have two different sizes. Typically, the size of the quantum dots range from about 3 nm to about 50 nm, and the size of the magnetic or paramagnetic nanoparticle ranges from about 70 nm to about 200 nm. In this embodiment, and by way of illustration only, one size of quantum dot is about 10 nm and the other size of quantum dot is about 25 nm. The sizes of the quantum dots are selected such that only one of the two sizes of quantum dot nanoparticles, and not the other size quantum dot nanoparticle, is susceptible to the external energy and increases in temperature, as described above for the magnetic or paramagnetic nanoparticle.

In one embodiment, the disclosed complex is activated by a light source that is implanted in the patient. This embodiment finds particular beneficial use for methods in which the quantum dot-nanoparticle is, or is likely to be, located at a body region or site that is less readily accessible or inaccessible to an external energy source, e.g. brain, spinal cord, etc. In one embodiment, an LED light source with a rechargeable battery is implanted in the patient. The LED provides a light pulse that activates the disclosed complex. In one embodiment, the light is transmitted by a fiber optic or a flexible silicone tube to a desired area(s).

In one embodiment, a fiber optic light source is implanted in a desired area of the brain, e.g., frontal lobe, parietal lobe, occipital lobe, temporal lobe, or cortex. In one embodiment, a fiber optic light source is implanted in a discrete area of the brain, e.g., basal ganglia including striatum, dorsal striatum, putamen, caudate nucleus, ventral striatum, nucleus accumbens, olfactory tubercle, globus pallidus, subthalamic nucleus; cerebellum including cerebellar vermis, cerebellar hemispheres, anterior lobe, posterior lobe, flocculonodular lobe, cerebellar nuclei, fastigial nucleus, globose nucleus, emboliform nucleus, dentate nucleus, and/or cortex including frontal lobe cortex and including primary motor cortex, supplementary motor cortex, premotor cortex, prefrontal cortex, gyri; parietal lobe cortex including primary somatosensory cortex (S1), secondary somatosensory cortex (S2), posterior parietal cortex, occipital lobe cortex including primary visual cortex (V1), V2, V3, V4, V5/MT; temporal lobe cortex including primary auditory cortex (A1), secondary auditory cortex (A2), inferior temporal cortex, posterior inferior temporal cortex; globus pallidus interna (GPi), caudal zona incerta, pallidofugal fibers, at an infarct site, at a scar tissue site, at a site in the spinal cord and/or peripheral nervous system.

In one embodiment, the disclosed complex need not necessarily be localized to the desired site for treatment, but the localized production of light causes activation of the complex at a desired site to treat the condition. The implanted LED/battery/fiber optic functions similar to an implanted cardiac pacemaker In one embodiment, a light source is external to the body with the end of the fiber optic accessible such that treatment can be performed outside a hospital setting, e.g., in a physician's office or in a medical outpatient facility. In one embodiment, the light source's controllable parameters, e.g., pulse frequency, pulse duration, pulse intensity, etc., can be controlled before or after implantation.

In one embodiment, a controller, either internal or external controls the light source's controllable parameters. The controller operates in a manner analogous to a cardiac pacemaker that regulates cardiac rhythm. It can be adjusted or regulated by a physician as needed, either through the skin or by exposing the implanted system at an appropriate and accessible location.

In one embodiment, an electrical sensor is provided with the implanted fiber optic, where the electrical sensor monitors conditions at the treatment site, such as electric potential, action potentials, etc. In embodiments, the electrical sensor is in communication with the controller such that the instructions provided by the controller to the light source, such as pulse frequency, pulse duration, pulse intensity, etc., may be adjusted by the controller based on the information from the electrical sensor.

In one embodiment, the electrical sensor is provided adjacent the implanted fiber optic source, e.g., along a side of the fiber optic source. For example, in one embodiment, the electrical sensor is implanted along a surface of a fiber optic tip, and ribbons, e.g., about 10 micron wide and spaced at 10 micron intervals, of graphene can be deposited/grown; the resulting graphene ribbons are then operatively connected to insulated wires. After implantation, graphene contacts the neuronal cells and terminates at various distances from the fiber optic tip. These graphene ribbons provide a feedback to the controller on the polarization status of the neuronal cells at the different depths of brain tissue. The graphene ribbons transmit the membrane polarization by the insulated wires attached to the graphene and to the controller, which is connected to the implanted light source, such as a light pulse generator (diode). The light source emits the software-controlled light pulses for stimulation of the area of the brain located near the fiber optic tip. One advantage of this embodiment is that the fiber optic device and light do not induce scar formation resulting in less or no tissue damage, and in contrast to currently used wires and devices to deliver electrical pulses. The light pulse achieves the desired results through activation of neuronal cells by activation of the disclosed complex, that is administered locally or systemically.

In one embodiment, the disclosed complex is injected locally immediately prior to placement of the fiber optic device though a cannula, under observation by magnetic resonance imaging (MRI). After the fiber optic is inserted in the cannula, the cannula is removed, leaving the wires connected to the fiber optic in the tissue. The exposed wires are connected to the controller that acts as a pulse receiver/generator. The results generated by the disclosed system can be evaluated using various methodologies, e.g., electroencephalogram (EEG), etc. In embodiments, the disclosed system creates a feedback for controlling the light-stimulated neuronal cells.

In embodiments, the implanted LED/battery-fiber optic is used with the disclosed quantum dot-nucleic acid complex for therapy of patients with Parkinson's disease, epilepsy, spinal cord injury, and neurological diseases affecting an action potential.

In one embodiment, a method for transferring IGF-I to a cirrhotic liver using the disclosed quantum dot-nucleic acid encoding IGF-I where IGF-I is under control of a liver-specific promoter, is provided. Results show improved liver function and reduced liver fibrosis. As used herein, IGF-I is used interchangeably with insulin-like growth factor I and somatomedin C and relates to a family of polypeptides characterized by showing insulin-like effects and insulin-like structure, sharing nearly 50% of amino acid homology with insulin.

In one embodiment, a method of expressing GLP-1 protein using the disclosed quantum dot-nucleic acid complex encoding GLP-1 or a GLP-1 analog provides therapy for type II diabetes. A GLP-1 analog, also encompassed, is defined as a molecule having a modification including one or more amino acid substitutions, deletions, inversions, or additions when compared with GLP-1. GLP-1 analogs known in the art include, e.g., GLP-1(7-34) and GLP-1(7-35), GLP-1(7-36), Val8-GLP-1(7-37), Gln9-GLP-1(7-37), D-Gln9-GLP-1(7-37), Thrl6-Lys18-GLP-1(7-37), and Lys18-GLP-1(7-37), disclosed in U.S. Pat. Nos. 5,118,666, 5,545,618, and 6,583,111. These compounds are the biologically processed forms of GLP-1 having insulinotropic properties.

In one embodiment, the disease for which the quantum dot-nucleic acid or gold nanoparticle coated with poly(lactic-co-glycolic acid) (PLGA) and cell penetrating peptides or activatable CPPs and Rock inhibitors-nucleic acid creating PNA/DNA/PNA complex is provided is characterized by dysregulation of the immune system. In this embodiment, the nucleic acid encodes a cytokine such as human interferon α 2b (hINFα) for therapy.

In one embodiment, using the disclosed methods, a tumor suppressor gene or genes is provided to a patient in need thereof, such as a cancer patient. A tumor suppressor gene as used herein means a nucleotide sequence that may inhibit a tumor phenotype depending on its expression in the cell or may induce apoptosis. Many tumors lack functional tumor suppressor genes that encode proteins that can arrest tumor growth and promote tumor cell apoptosis. For example, the p53 protein arrests the cell cycle following DNA damage and is also involved in apoptosis. Efficient delivery and expression of the wild-type p53 gene cause regression of established human tumors, prevent growth of human cancer cells in culture, and renders malignant cells from human biopsies non-tumorigenic in nude mice. The p53 gene has been combined with standard therapies such as chemotherapy and radiotherapy with positive effect. In one embodiment, a method of therapy for cancer in a patient is provided. In this embodiment, the quantum dot-nucleic acid or gold nanoparticle coated with poly(lactic-co-glycolic acid) (PLGA) and cell penetrating peptides or activatable CPPs and Rock inhibitors-nucleic acid creating PNA/DNA/PNA complex encoding p53 is provided to a patient, thereby providing therapy to the patient. Besides the p53 gene, other tumor suppressor genes include APC gene, DPC-4/Smad4 gene, BRCA-1 gene, BRCA-2 gene, WT-1 gene, retinoblastoma gene (Lee et al., Nature, 329: 642 (1987)), MMAC-1 gene, adenomatouspolyposis coil protein, deleted in colorectal cancer (DCC) gene, MMSC-2 gene, NF-1 gene, nasopharyngeal carcinoma tumor suppressor gene that maps at chromosome 3p21.3, MTS1 gene, CDK4 gene, NF-1 gene, NF-2 gene and/or VHL gene.

Other therapeutic genes useful for the disclosed method include those that encode enzymes, blood derivatives, hormones, lymphokines such as interleukins, interferons, tumor necrosis factor, etc., growth factors, neurotransmitters or their precursors or synthetic enzymes, trophic factors namely BDNF, CNTF, NGF, IGF, GMF, αFGF, βFGF, NT3, NT5, HARP/pleiotrophin, etc., apolipoproteins such as ApoAI, ApoAIV, ApoE, etc., dystrophin or a minidystrophin, the CFTR protein associated with cystic fibrosis, intrabodies, tumor-suppressing genes such as p53, Rb, Rap1A, DCC, k-rev, etc., genes encoding coagulation factors such as factors VII, VIII, IX, genes participating in DNA repair, suicide genes defined as genes whose products cause cell death, e.g., thymidine kinase (HS-TK), cytosine deaminase, etc., pro-apoptic genes, prodrug converting genes defined as genes encoding enzymes that convert prodrugs to drugs, and anti-angiogenic genes or alternatively, genes such as VEGF that promote angiogenesis.

The nucleic acid portion of the quantum dot-nucleic acid complex can also be used in gene silencing. Such gene silencing may be useful in therapy to switch off aberrant gene expression or studies to create single or genetic knockout models. Such nucleic acid is typically provided in the form of siRNAs. For example, RNAi molecules including siRNAs can be used to knock down DMPK with multiple CUG repeats in muscle cells for myotonic dystrophy therapy. In other examples, plasmids expressing shRNA that reduce the mutant Huntington gene (htt) responsible for Huntington's disease can be delivered. Other target genes include BACE-1 for the therapy of Alzheimer's disease. Some cancer genes may also be targeted with siRNA or shRNAs, such as ras, c-myc and VEGFR-2. Brain targeted siRNA may be useful in silencing BACE-1 in Alzheimer's disease, silencing of α-synuclein in Parkinson's disease, silencing of htt in Huntingdon's disease, and silencing of neuronal caspase-3 used in therapy of stroke to reduce ischemic damage.

In one embodiment, the nucleic acid is an RNA interference (RNAi), small interfering RNA or short interfering RNA (siRNA), short hairpin RNA (shRNA) molecule, or miRNA which is a RNA duplex of nucleotides targeted to a nucleic acid sequence of interest, e.g. huntingtin. As used herein, siRNA is a generic term that encompasses the subset of shRNAs and miRNAs. An RNA duplex refers to the structure formed by the complementary pairing between two regions of a RNA molecule. siRNA is targeted to a gene in that the nucleotide sequence of the duplex portion of the siRNA is complementary to a nucleotide sequence of the targeted gene. In embodiments, siRNAs are targeted to the sequence encoding ataxin-1 or huntingtin. In embodiments, the length of the duplex of siRNAs is less than 30 base pairs. In embodiments, the duplex can be 29, 28, 27, 26, 25, 24, 23, 22, 21, 20, 19, 18, 17, 16, 15, 14, 13, 12, 11 or 10 base pairs in length. In embodiments, the length of the duplex is 19 to 25 base pairs in length. In embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In embodiments the loop is 5, 6, 7, 8, 9, 10, 11, 12, 13, 14, 15, 16, 17, 18, 19, 20, 21, 22, 23, 24 or 25 nucleotides in length. In embodiments, the loop is 18 nucleotides in length. The hairpin structure can also contain 3′ and/or 5′ overhang portions. In embodiments, the overhang is a 3′ and/or a 5′ overhang 0, 1, 2, 3, 4 or 5 nucleotides in length. In one embodiment, the various forms of RNA such as microRNA, RNA interference, RNAi, and siRNA are designed to match the RNA copied from a defective gene, thereby inhibiting or diminishing production of the abnormal protein product of that gene.

In some embodiments, it may be useful to assess, monitor, track, evaluate location, evaluate stability, etc. of the particles conjugated or otherwise associated with a moiety as previously described. In these embodiments, the particles are tagged with a recognition moiety to provide a signal, and may themselves be conjugated to another biologically active moiety, e.g., DNA, RNA, peptide, protein, antibody, enzyme, receptor, etc., as known to one skilled in the art. Tagging may be effected via a covalent bond with a amide, thiol, hydroxyl, carbonyl, sulfo, or other such group on the biologically active moiety, as well known to one skilled in the art.

While each solar cell particle is oriented, in one embodiment, the plurality of particles provided in the body are not uniformly directionally oriented, nor do they require a backing layer to maintain orientation or position. They have a positive-negative (P-N) junction diode and may be constructed as either negative-intrinsic-positive (NIP) or positive-intrinsic-negative (PIN), as known to one skilled in the art. In one embodiment, where the nanoparticles are inserted into the cell membrane, the differential coating of portions of the particle with hydrophobic and hydrophilic materials can result in an orientation of the particles in the cell membrane.

As an example, p-type silicon wafers, and doped p-type silicon wafers to form n-type silicon wafers, are contacted to form a p-n junction. Electrons diffuse from the region of high electron concentration, the n-type side of the junction, into the region of low electron concentration, the p-type side of the junction. When the electrons diffuse across the p-n junction, they recombine with an electron deficiency (holes) on the p-type side. This diffusion of carriers does not happen indefinitely however, because of the electric field created by the imbalance of charge immediately either side of the junction which this diffusion creates. Electrons from donor atoms on the n-type side of the junction cross into the p-type side, leaving behind the (extra) positively charged nuclei of the group 15 (V) donor atoms such as phosphorous or arsenic, leaving an excess of positive charge on the n-type side of the junction. At the same time, these electrons are filling holes on the p-type side of the junction and are becoming involved in covalent bonds around the group 13 (III) acceptor atoms such as aluminum or gallium, making an excess of negative charge on the p-type side of the junction. This imbalance of charge across the p-n junction sets up an electric field which opposes further diffusion of charge carriers across the junction. The region where electrons have diffused across the junction is called the depletion region or the space charge region because it no longer contains any mobile charge carriers. The electric field which is set up across the p-n junction creates a diode, allowing current to flow in only one direction across the junction. Electrons may pass from the n-type side into the p-type side, and holes may pass from the p-type side to the n-type side. Because the sign of the charge on electrons and holes is opposite, current flows in only one direction. Once the electron-hole pair has been created by the absorption of a photon, the electron and hole are both free to move off independently within a silicon lattice. If they are created within a minority carrier diffusion length of the junction, then, depending on which side of the junction the electron-hole pair is created, the electric field at the junction will either sweep the electron to the n-type side, or the hole to the p-type side.

One embodiment of the invention uses nanocrystals of semiconductor material referred to as quantum dots (Evident Technologies, Troy NY; Oceano NanoTech, Springdale AK). Nanocrystal solar cells are solar cells based on a substrate with a coating of nanocrystal. The nanocrystals are typically based on silicon, CdTe or CIGS and the substrates are generally silicon or various organic conductors. Quantum dot solar cells are a variant of this approach. These have a composition and size that provides quantum properties between that of single molecules and bulk materials, and are tunable to absorb light over the spectrum from visible to infrared energies. Their dimensions are measured in nanometers, e.g., diameter between about 1 nm to about 100 nm. When combined with organic semiconductors selected to have the desired activation properties, they result in particles with selectable features. The particles can also have passive iron oxide coatings with or without polyethylene glycol coatings or positive charge coatings as commercially provided. Quantum dot solar cells take advantage of quantum mechanical effects to extract further performance.

Nanocrystals are semiconductors with tunable bandgaps. The quantum dot nanocrystal absorption spectrum appears as a series of overlapping peaks that get larger at shorter wavelengths. Because of their discrete electron energy levels, each peak corresponds to an energy transition between discrete electron-hole (exciton) energy levels. The quantum dots do not absorb light that has a wavelength longer than that of the first exciton peak, also referred to as the absorption onset. Like other optical and electronic properties, the wavelength of the first exciton peak, and all subsequent peaks, is a function of the composition and size of the quantum dot. Smaller dots result in a first exciton peak at shorter wavelengths.

The quantum dots may be provided as a core, with a shell or coating of one or more atomic layers of an inorganic wide band semiconductor. This increases quantum yield and reduces nonradiative recombination, resulting in brighter emission provided that the shell is of a different semiconductor material with a wider bandgap than the core semiconductor material. The higher quantum yield is due to changes in the surface chemistry of the core quantum dot. The surface of nanocrystals that lack a shell has both free (unbonded) electrons, in addition to crystal defects. Both of these characteristics tend to reduce quantum yield by permitting nonradiative electron energy transitions at the surface. A shell reduces opportunities for nonradiative transitions by giving conduction band electrons an increased probability of directly relaxing to the valence band. The shell also neutralizes the effects of many types of surface defects.

The quantum dots may respond to various wave lengths of electromagnetic radiation, i.e., visible, invisible, ultrasound, microwaves. The quantum dots respond by emitting an electrical potential or fluoresce when exposed to electromagnetic radiation. The quantum dots may be made, or self-assembled, from CdSe and a shell of zinc gallium arsenide, indium gallium selenide, or cadmium telluride. Luminescent semiconductor quantum dots such as zinc sulfide-capped cadmium selenide may be covalently coupled to biomolecules for use in ultrasensitive biological detection. These nanometer-sized conjugates are water-soluble and biocompatible.

Quantum dots, organic quantum dots, or solar cells may be made from organic molecules such as organic nanocrystal solar cells, polymers, crystalline forms of carbon such as fullerenes, etc. In one embodiment, the crystalline form of carbon is fullerene. In one embodiment, the crystalline form of carbon is graphene. In one embodiment, the crystalline form of carbon is a carbon nanotube. Embodiments also include combinations of such crystalline forms of carbon. Quantum dots may be coated with organic molecules, biocompatible proteins, peptides, phospholipids, or biotargeted molecules etc., or covalently attached to polyethylene glycol polymers (i.e., they may be PEGylated) to last longer. In one embodiment, hybrid quantum dots including but not limited to graphene/zinc oxide (ZnO) and reduced graphene oxide, or plasmonic nanoparticles coated with reduced graphene oxide, dextran-reduced graphene oxide, etc. may be used. In embodiments, ZnO is added to graphene quantum dots or to a combination of graphene particles and/or carbon nanotubes with a ZnO nanowire or nanorod using an electron gun. In embodiments, particularly those using light to stimulate the described particle, ZnO is useful because it prevents light reflecting off the particle surface, i.e., it serves as an anti-reflective coating, and provides a more efficient quantum dot compared with graphene or a carbon nanotube alone. ZnO additionally has the benefit of being an antibacterial compound and thus can be utilized for transporting biomolecules, such as DNA, along with other polymers; these may contribute a further therapeutic function and/or to the bio-compatibility of the disclosed complex.

In embodiments using a graphene and/or graphene oxide nanoparticle, optionally containing additional therapeutic or biocompatibility enhancing molecules such as peptides, Rock inhibitors, etc., the application of the disclosed nanoparticle enhances neuronal growth. For example, the disclosed nanoparticle may be administered in response to brain and/or spinal cord injury, during ophthalmic LASIK surgery prior to closure of the corneal flap and/or after such surgery to stimulate neuronal growth, to neural tissue affected by Alzheimer's disease, or into the eye in genetic diseases of the eye, or ischemia leading to possible infarction and ischemic stroke, to damaged peripheral nerves, etc. to result in enhanced neuronal growth. The disclosed nanoparticle can be applied, e.g., on the corneal stroma, on an exposed wound, or on damaged nerves, as a drop or injected locally, or can be applied on or with a biocompatible substrate at a neuronal injury or infarction.

For example, brain-derived neurotrophic factor (BDNF) or Bromidin may be administered locally in combination with the inventive nanoparticle/quantum dot embodiments. These may be further provided with agents that enhance neurite outgrowth, e.g., myelin basic protein (MBP), valproic acid, ketamine, donepezil hydrochloride, thymosin β10, thymosin α1, choline acetyl esterase, etc. The therapeutic molecules may be contained in or on the quantum dot and enhance local neurite growth and promote neuron functional recovery.

These quantum dots, or devices containing quantum dots, are amenable to large scale production. They may be built from thin films, polymers of organic semiconductors. These devices differ from inorganic semiconductor solar cells in that they do not rely on the large built-in electric field of a PN junction to separate the electrons and holes created when photons are absorbed. The active region of an organic device consists of two materials, one which acts as an electron donor and the other as an acceptor. The short excitation diffusion lengths of most polymer systems tend to limit the efficiency of such devices. However, quantum dots can be used for cell membrane stimulation.

The quantum dots can be made to respond to various wavelengths of light (visible and invisible). In one embodiment they are coated with organic molecules. In one embodiment, they are completely organic. In one embodiment, they are PEGylated to last longer. In one embodiment, they are coated to be attracted to certain receptors or stay only on the cell surface.

In one embodiment, quantum dots, such as graphene nanoparticles, can be made into graphene transistor with a very large cut-off frequency, e.g., greater than 20 gigahertz, greater than 40 gigahertz, or up to 100 gigahertz. In one embodiment, wafer-scale, epitaxially grown graphene is used. Uniform and high-quality graphene wafers can be synthesized by thermal decomposition of a silicon carbide (SiC) substrate. The graphene transistor itself may use a metal top-gate architecture and a gate insulator stack involving a polymer and a high dielectric constant oxide. In embodiments, the gate length can be varied as desired. In one embodiment, the gate length is about 240 nanometers. In one embodiment, a one-atom-thick, two-dimensional metamaterial is produced by controlling the conductivity of sheets of graphene, a single layer of carbon atoms, by manipulating electromagnetic (EM) acoustic waves in the infrared spectrum. Applying direct voltage to a sheet of graphene by a ground plate parallel to a sheet of graphene, the conductivity of the graphene can be altered by varying the voltage or the distance between the ground plate and the graphene sheet. The sheet of graphene can have two areas that have different conductivities: one that can support an EM wave, and one that cannot support an EM wave. The boundary between the two areas acts as a wall, capable of reflecting a guided EM wave on the graphene. In embodiments, a third region may be created that can support a wave, surrounded by two regions that cannot support a wave, producing a “waveguide” that functions as a one-atom-thick fiber optic cable to carry signals. In another embodiment, another non-supporting region is added to bifurcate the waveguide, splitting it in two. In embodiments, as previously described, the one-atom-thick fiber optic cable may be used to stimulate cells and/or to detect changes in the stimulated cells.

Bioelectrical signals exist in all cells and play an important role in allowing the cells to communicate with each other. Quantum dots can facilitate these signal transmission between the cells, such as through cell membranes and their membrane potentials, thereby maintaining normal function in the tissue which include cell survival and growth, individually or collectively. Quantum dots can enhance regeneration of the cells. Quantum dots can enhance neural axons and enhance the wound healing process.

Cell activity relates to depolarization and re-polarization of the cell membrane. Quantum dots and/or semiconductor nanowires conjugated with Rock inhibitors can regulate polarization and depolarization and thus enhance the action potential of the membrane. Lack of cell activity leads to cell atrophy. Similarly, loss of the cell membrane potential causes cell degeneration and atrophy. The therapeutic effects of particle administration are achieved by the effects that the particles exert on membrane potential when stimulated, e.g. light, photoelectrical, ultrasound, etc. In the eye and in the nervous system, particles can be stimulated (e.g., through the cornea, sclera or skull etc. for the brain, spinal cord, and nerves), thus enhancing or maintaining the cell membrane potential (e.g., nerve cell, glial cells, astrocytes, etc.). This process preserves the function of such cells (nerve cells, glial cells, astrocytes, etc.) by maintaining their membrane potentials, thus maintaining cell viability and function.

In one embodiment, the method and concept is applied to the eye. In one embodiment, the method and concept is applied to the brain and spinal cord nerve cells and axons. In this embodiment, the method is used to enhance or stimulate regrowth of nerve cells, axons, and/or other brain and spinal cord tissue. In one embodiment the method is applied to the heart.

In one embodiment, the effects of the particles on the cells can be enhanced by combining quantum dots with growth factors. Such growth factors are known to one skilled in the art, and include but are not limited to nerve growth factors, glial growth factors, placenta growth factor, etc. In one embodiment the effects of the particles on the cells can be enhanced by administering and/or regulating quantum dots essentially simultaneously with certain pharmaceuticals or agents, including but not limited to TAXOL®, carbonic anhydrase inhibitors, etc. Quantum dots and/or semiconductor nanowires, when activated by light, enhance drug penetration through the cell membrane. This can be used therapeutically in combination with many medications which may not penetrate the cell membrane easily because of their chemical structures. However, this concept can be used also in conjunction with antibiotics, antifungal agents, etc. to kill the organism that caused skin or mucosa ulcers resisting therapy.

The treatment can be done easily by topically applying the particles along with the appropriate medication and using light to activate the particles. The method of delivery to the eye may be by injection, eye drops, ointments, sprays or other applications to treat an optic nerve. The method of delivery to the brain may be by injection of the particles into cerebrospinal fluid, brain ventricles, intra-ocularly, or administration by nasal sprays or drops. The method of delivery to the skin or mucosa, e.g., nasal mucosa, is by spraying. Most of these applications avoid possible systemic side effects. The size of the particles allows them to easily diffuse into tissues. For neural applications other than the eye, quantum dots and/or semiconductor nanowires, either conjugated or associated with a drug, e.g., Rock inhibitors, and/or administered without a drug or other agent, are administered by any route of delivery including but not limited to local, systemic, injection in the CNS, topical application by nasal mucosal routes, e.g., spray, drops, to regulate the nasal olfactory nerve, or localized injection in the vicinity of the peripheral nerves or ganglions, etc. In these cases, the stimulation of the nanoparticles can be done through the nasal mucosa or externally through the skin at the base of the nose to stimulate the brain.

In any of the disclosed embodiments, the nanoparticles may themselves be rendered capable of enhanced penetration into a cell by providing the nanoparticles in formulations, with agents, in composition, etc. that enhance cell penetration, having all requirements for therapeutic and biomedical uses such as biocompatibility, biodegradability (bioerodability and/or bioabsorbability), stability, and low toxicity. Such nanoplatform carriers include micelles, liposomes, dendrimers, and solid, lipid, metallic, semiconductors, peptide, and polymeric nanoparticles as subsequently described.

In one embodiment nanoparticles, e.g., quantum dots, are biocompatible or rendered biocompatible, e.g., by coating with a biocompatible polymer such as (poly)ethylene glycol (PEG) moieties, e.g., quantum dot-DNA-coated polymer, or any other polymer or combinations of polymers meeting these criteria. Exemplary such polymers also include, but are not limited to, polymers or co-polymers of poly(ester amide), poly-hydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3 hydroxyheptanoate), and poly(3 hydroxyoctanoate), poly(4-hydroxyalknaote) such as poly(4 hydroxybutyrate), poly(4-hydroxyvalcrate), poly(4-hydroxyhexanote), poly(4hydroxyheptanoate), poly(4-hydroxyoctanoate) including 3-hydroxyalknnoate or 4-hydroxyalkanoate monomers, polyesters, poly(D,L-lactide), poly(L-lactide), polyglycolide, poly(D,L-lactide-co-glycolide), poly(lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives, poly(tyrosine ester) and derivatives, poly(imino carbonates), poly(glycolic acid-co-trimethylene carbonate), polyphosphoester, polyphosphoester urethane, poly(amino acids), polycyanoacrylates, poly(trimethylene carbonate) poly(iminocar bonate), polyurethanes, polyphosphazenes, silicones, polyesters, polyolefins, polyisobutylene and ethylene-alphaolefin copolymers, acrylic polymers and copolymers, vinyl halide polymers and copolymers (e.g., polyvinyl chloride), polyvinyl ethers such as polyvinyl methyl ether, polyvinylidene halides such as and polyvinylidene chloride, polyacrylonitrile, polyvinyl ketones, polyvinyl aromatics, such as polystyrene, polyvinyl esters such as polyvinyl acetate, copolymers of vinyl monomers with each other and olefins such as ethylene-methyl methacrylate copolymers, acrylonitrile-styrene copolymers, ABS resins, and ethylene-vinyl acetate copolymers, polyamides such as Nylon 66 and polycapro-lactam, alkyd resins, polycarbonates, polyoxymcthylenes, polyimides, polyethers, poly(glyceryl sebacate), poly(propylene fumarate), epoxy resins, polyurethanes, rayon, rayon-triacetate, cellulose acetate, cellulose butyrate, cellulose acetate butyrate, cellophane, cellulose nitrate, cellulose pro-pionate, cellulose ethers, carboxymethyl cellulose, polyethers such as PEG, copoly(ether esters) (e.g. PEO/PLA); polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as hydroxyethyl methacrylate (HEMA), hydroxypropyl methacrylate (HEMA), hydroxypropyl methacrylamide, PEG acrylate (PEGA), PEG methacrylate, 2-ethacryloyloxyethylphosphoryl-choline (MPC) and n-vinyl pyrrolidone (VP), carboxylic acid bearing monomers such as methacrylic acid (MA), acrylic acid (AA), alkoxymethacrylate, alkoxyacrylatc, and 3-trimethylsilylpropyl methacrylate (TMSPMA), poly(styrene-isoprene-styrene)-PEG (SIS-PEG), polystyrene-PEG, polyisobutylene PEG, polycaprolactone-PEG (PCL-PEG). PLA-PEG, poly(methyl methlcrylate)-PEG (PMMA-PEG), polydimethylsiloxane-co-PEG (PDMS-PEG), poly(vi nylidene fluoride)-PEG (PVDF-PEG), PLURONIC™ surfactants (polypropylene oxide-co-polyethylene glycol), poly (tetramethylene glycol), hydroxy functional poly(vinyl pyrrolidone).

These also include nanoparticles, e.g., quantum dots, that are biocompatible or rendered biocompatible using peptides such as arginine-rich peptides, trans-activation transcriptional activator (Tat) peptides, biocompatible short peptides of naturally occurring amino acids that have the optical and electronic properties of semiconductor nano-crystals, e.g., short peptides of phenylalanine with particles consisting of both inorganic or organic materials. These also include nanoparticles, e.g., quantum dots, that are biocompatible or rendered biocompatible using biocompatible mono- or bilayers of phospholipid, liposomes, etc. These also include nanoparticles, e.g., quantum dots, that are biocompatible or rendered biocompatible using a specific agent and/or coating to the nanoparticles renders them specific, e.g., a protein coating to direct nanoparticles to attach to certain cell membranes, e.g., a member of a streptavidin-biotin pair, an immunoglobulin, a member of a cell-specific antibody-antigen pair, etc. These also include nanoparticles, e.g., quantum dots, to enter a cell to increase the membrane potential of the cells to which they come into contact, that are biodegradable either entirely or partially, that are non-biodegradable, and/or that are a combination of organic and metallic quantum dots. These include nanoparticles, nanotubes, nanowires, nanocrystals such as cadmium/selenium (Cd/Se), and particular types of each, e.g., graphene quantum dots, graphene-oxide quantum dots, graphene-zinc oxide quantum dots, graphene nanotubes, and/or carbon nanotubes.

As previously disclosed, all the above embodiments use compounds that are collectively termed nanoparticles. The nanoparticles with enhanced cell penetration may also be included in or coated on a bioabsorbable or non-bioabsorbable but biocompatible polymer structured or configured as a fiber, a tube, a substantially two-dimensional structure, or a three-dimensional structure to fit any anatomical or physiological site, configured as any desirable length or size to maintain its position with respect to an anatomical and/or physiological location, e.g., the eye. Upon activation, cells and adjacent tissue may undergo excitation, which is further tunable, e.g., parts of the polymer, e.g., the front and back sides of a substantially two-dimensional structure, having different particles to effect target cells, adjacent cells, etc.

Nanoplatforms may be developed of such carriers with both superior biocompatibility and superior penetrability. Pan et al., ACS Appl. Mater. Interfaces 5 (2013) 7042, disclose one such example of a family of silica crosslinked micelles that are biocompatible, luminescent, and stable; specifically, silica crosslinked pluronic F127 (difunctional block copolymer surfactant) micelles loaded with decyl capped silicon nanoparticles. Erogbogbo et al. (Integr. Biol. 5 (2013) 144) disclose another such example of a biocompatible silicon quantum dot F127 micelle with multiple silicon quantum dots incorporated into a micelle core having gold deposited on the nanostructure surface to provide light scattering properties facilitating imaging, while the silicon nanocrystals maintain photoluminescence.

Nanoparticles such as quantum dots that are coated on their surface with, encapsulated by, or otherwise associated with polymers, such as any of those previously described, have enhanced cell penetrating ability, as previously disclosed. Synthesis, fabrication, modifications, are possible, e.g., Tomczak et al., Progress in Polymer Science, 34 (2009) 393.

As one example, PEG grafted polyethylenimines (PEI) encapsulate and solubilize luminescent quantum dots through direct ligand-exchange reactions via positive charges and a proton sponge effect. PEG improves nanoparticle stability and biocompatibility, as previously disclosed, and reduces PEI toxicity to cells, as well as facilitating cell penetration. Duan and Nle, J. Am. Chem. Soc. 129 (2007) 3333.

As one example, nanoparticles such as quantum dots can be coated with proton-absorbing polymers, termed proton sponges, to facilitate siRNA delivery by enhanced cell penetration as well as other means. Yezhelyev et al., J. Am. Chem. Soc. 130 (2008) 9006.

As one example, the linear polysaccharide chitosan, in the form of beads, gels, sponges, membranes, scaffolds, etc. may be used as PEG-chitosan and/or folate-chitosan quantum dots. Rajan and Raj, I.Re.CH.E. 5 (2013) 145. As one example, folate conjugated, PEG-coated quantum dots specifically recognized and were internalized by folate receptors that were overexpressed in certain cancer cells, i.e., specific receptor-mediated cellular internalization. Song et al., Clincal Chemistry 55 (2009) 955. As one example, PEG-conjugated chitosan derivatives were synthesized and demonstrated narrow size distribution, good water solubility, low cytotoxicity. Lv et al., Chemical Papers 67 (2013) 1404.

Various coatings and encapsulation conditions, components, linkage types, etc., i.e., nanoparticle architecture, can assist in regulating nanoparticle penetration effects and parameters under various conditions. Smith et al., Advanced Drug Delivery Reviews 60 (2008) 1226. As one example, at least two of the same or different, i.e., hybrid, nanoparticles such as quantum dots can be encapsulated in amphiphillic block copolymer micelles that are crosslinked and thus form a shell surrounding the plurality of nanoparticles. Kim and Taton, Langmuir, 23 (2007) 2196. As one example, nanoparticles such as quantum dots can be encapsulated in carboxylated Pluronic (i.e., poly(ethylene oxide) (PEO) and poly(propylene oxide) (PPO) with a PEO—PPO-PEO structure) 127 triblock, forming a polymeric micelle with efficient cell penetration. Liu et al. Theranostics 2 (2012) 705.

Nanoparticles such as quantum dots may be conjugated with various physiologic biomolecules to enhance or facilitate cell penetration. Examples of such compounds include, but are not limited to, carbohydrates, cholesterol, glutathione (e.g., taking advantage of amino functionalities), collagen, chitosan, alginate, fibrin, fibrinogen, cellulose, starch, collagen, dextran, dextrin, fragments and derivatives of hyaluronic acid, heparin, fragments and derivatives of heparin, glycosamino glycan (GAG), GAG derivatives, polysaccharide, elastin, and biotin, proving good stability and cell viability, e.g., Jin et al., Int. J. Mol. Sci. 9 (2008) 2044.

One example of this enhanced penetration is quantum dots conjugated or otherwise associated with cell penetrating agents such as cell penetrating peptides (CPP) and activatable-cell penetrating peptides (ACPP).

Cell-penetrating peptides (CPP) and activatable cell-penetrating peptides (ACPPs) that are labeled with fluorescent polycationic CPP coupled by a cleavable linker to a neutralizing peptide have been developed and utilized to visualize tumors during surgery. ACPP conjugated to dendrimers (ACPPDs) and gadolinium chelates can allow MRI visualization of whole body tumors, permitting therapy if the magnetic or gadolinium nanoparticles are labeled with ACPPD.

One embodiment is a composition of a nanoparticle such as a quantum dot that is conjugated to an activatable cell penetrating peptide (ACPP). The quantum dot or nanoparticle may be cleavably conjugated to the ACPP by, e.g., a linker (e.g., ethylene glycol moiety, PEG moiety). In one embodiment, the quantum dot or nanoparticle is labeled with a label such as a fluorescent moiety, chemiluminescent moiety, etc. In one embodiment, the ACPP is labeled with a polycationic cell-penetrating peptide (CPP). ACPP and CPP may be naturally-occuring or artificially constructed protein segments (<30 amino acids) rich in arginine, lysine, cysteine, histidine, ornithine, etc.; preferably α-helices and about 17-amino acids. The ACPP and CPP may include a penetration accelerating peptide sequence (Pas) or an INF7 fusion peptide sequence. CPP and/or ACCP can be linked to cargoes either covalently or noncovalently, or can use block copolymers to form various kinds of micelles. Liu et al., endocytic trafficking of Nanoparticles Delivered by Cell-penetrating peptides Comprised of Nona-arginine and a Penetration Accelerating Sequence, PLOS ONE 8 (2013) e67100; Liu et al., Intracellular Delivery of Nanoparticles and DNAs by IR9 Cell-penetrating Peptides, PLOS ONE 8 (2013) e6405; Liu et al., Cell-Penetrating Peptide-Functionalized quantum Dots for Intracellular Delivery, J. Nanosci Nanotechnol 10 (2010) 7897; Liu et al., Cellular Internalization of Quantum Dots Noncovalently Conjugated with Arginine-Rich Cell-Penetrating Peptides, J. Nanosci Nanotechnol 10 (2010) 6534; Xu et al., Nona-Arginine Facilitates Delivery of Quantum Dots into Cells via Multiple Pathways J. Biomedicine and Biotechnology volume 2010, Article ID 948543, 1-11. Exemplary but non-limiting ACPP and CPP include transportan, penetratin, TAT, VP22, MAP, KALA, ppTG20, proline-rich peptides, MPG-derived peptides, Pep-1, nona-arginine, and the carboxy-terminal tail of TFPI-2, polyproline helices having cationic amino acids and/or cationic-functionalized amino acids within the helix (e.g., guanidinium-functionalized proline), a synthesized oligoarginine cell penetrating peptide (based on the HIV-1 Tat protein motif) bearing a terminal polyhistidine (His8) tract facilitated transmembrane quantum dot delivery (Delehanty et al., Bioconjug Chem 17 (2006) 920). Other cell penetrating agents include oligoguanidinium scaffolds, cationic-functionalized calixarenes, or cyclodextrins, e.g., arginine-functionalized calixarene). For example, CPP comprising a INF7 fusion peptide and nona-arginine produces IR9/QD and IR9/DNA complexes, IR9, IR9/QD and IR9/DNA. Handbook of Cell Penetrating Peptides, Langel (Ed), 2007 (Second Edition) CRC/Taylor & Francis).

One example of this enhanced penetration uses genes capable of modifying cell polarization and, potentially, creating an action potential upon specific prompts. Such genes encode, e.g., a cell membrane ion channel protein or transporter, e.g., a G protein-coupled receptors. Specific examples include genes encoding opsin family members such as rhodopsin, photopsins, halorhodopsin, genes encoding neurotransmitters such as glutamate/aspartate transporters, GABA transporters, glycine transporters, monoamine transporters such as dopamine transporter, norepinephrine transporter, serotonin transporter, and vesicular monoamine transporters. Upon exposure to light of a specific wavelength, quantum dots coated or otherwise associate with organic or non-organic biodegradable compounds may be used. Such agents include silicon, porous silicon, aliphatic biodegradable polymers, etc. the quantum dots range from about 1 nm to 200 nm in one embodiment, and range from about 1 nm to 10 nm in another embodiment. The genes, once in the nucleus, undergo transcription and translation into the specific proteins or protein channels. This embodiment is described in, e.g., Narayanan et al., Scientific Reports 3, article number 2184, doi: 10.1039/srep02184 describing a mimic of microtubule mediated protein transport using designed biotinylated peptides with microtubule-associated sequences (MTAS) and a nuclear localization signaling (NLS) sequence, conjugating with streptavidin-coated CdSe/ZnS quantum dots to enhance endosomal escape and promote targeted nuclear delivery into mesenchymal stem cells by microtubules. This embodiment is also described in Ho et al., J. Vis Exp. 30 (2009) 1432, describing a specific quantum dot-labeled DNA complex (Cy5) that forms by electrostatic self-assembly, facilitating DNA cellular update and protecting against DNA degradation and monitored using combined quantum dot-FRET and microfluidics.

Agents may be linked to, associated with, complexed or conjugated with nanoparticles using linking agents and methods known in the art. These include, but are not limited to, the following: amino groups, carboxyl groups, S—S deprotected sulfhydril groups in biomolecules, e.g., bis(succinimide derivative conjugation, maleimidosuccinimide/succinimidylpyridyldithio/-halosuccinimide derivative conjugation, N-(4-maleimido-phenyl) isocyanate conjugation, carbodiimide conjugation, sulfosuccinimidylsuberyl linkage, synthetic tripyrrole-peptide linkage, NHS-esters and other esters, etc. Cleavage of the linkers by specific proteases, e.g., matrix metalloproteinase-2, dissociates the polyanion and enables arginine-rich CPPs to enter cells.

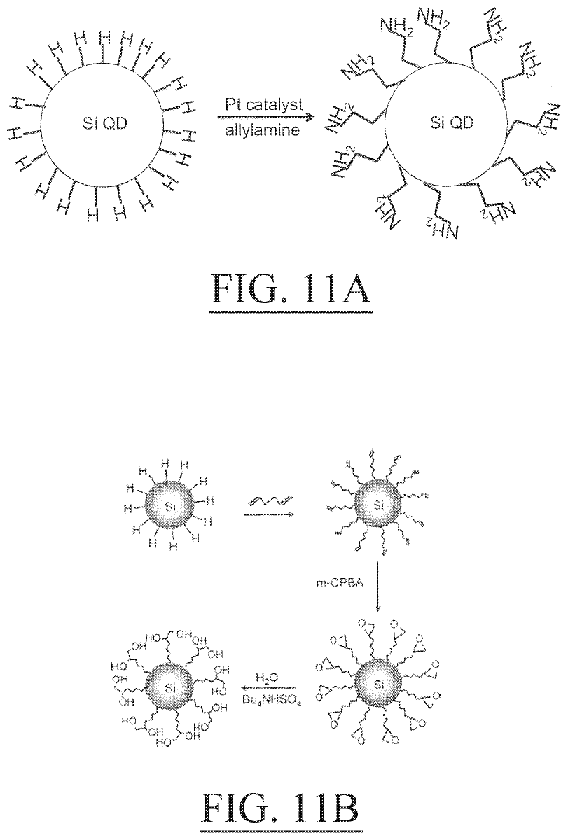

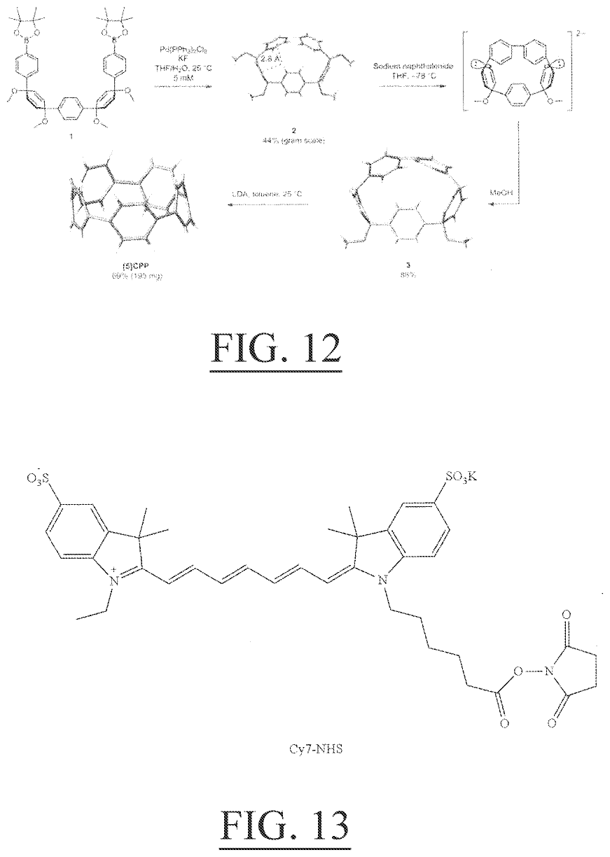

FIGS. 11-13 show various non-limiting embodiments of selected structures. FIGS. 11A-B schematically show an activated biodegradable silicone or luminescent quantum dot. FIG. 12 schematically shows synthesis of a cell-penetrating peptide (CPP). FIG. 13 shows the chemical structure of an activated fluorescent dye.

In one embodiment, the inventive method is used in a patient with a neurological disorder. While described in detail for use in a patient with epilepsy, which is a common neurological disorder requiring treatment, the inventive method is not so limited and encompasses any neurological disorder of the central and/or peripheral nervous system. Epilepsy is thus used an exemplary but non-limiting embodiment of use of the method.

Epilepsy is a chronic condition that transiently affects about 50 million individuals. It is not a single disorder, but instead is a group of syndromes with vastly divergent symptoms. Its unifying and diagnostic feature is episodic abnormal electrical activity in the brain that results in seizures. These seizures are transient, recurrent, and unprovoked; signs and/or symptoms of abnormal, excessive, or synchronous neuronal activity in the brain. All seizures involve loss of consciousness; types of seizures are characterized according to their effect on the body. These include absence (petit mal), myoclonic, clonic, tonic, tonic-clonic (grand mal), and atonic seizures.

Some forms of epilepsy are confined to particular stages of childhood. In children, epilepsy may result from genetic, congenital, and/or developmental abnormalities. In adults over 40, it may result from tumors. At any age, it may result from head trauma and central nervous system infections. Post-traumatic epilepsy (PTE) is a form of epilepsy that results from brain damage caused by physical trauma to the brain: traumatic brain injury (TBI). An individual with PTE suffers repeated post-traumatic seizures (PTS) more than a week after the initial injury. PTE can also occur after infectious diseases involving the CNS or peripheral nerves.

Epilepsy is usually controlled, but not cured, with medication, although surgery is sometimes needed. Therapeutic agents include (a) sodium channel blockers (voltage dependent), (b) calcium channel blockers (T-type), (c) potentiators of GABA (inhibitory), and (d) those that decrease excitatory transmission (glutaminic).

Some medication, administered daily, may prevent seizures altogether or reduce their frequency. Such medications, termed anticonvulsant drugs or antiepileptic drugs (AEDs), include valproate semisodium (Depakote, Epival), valproic acid (Depakene, Convulex), vigabatrin (Sabril), and zonisamide (Zonegran). A problem is that all have idiosyncratic and non-dose-dependent side effects. Thus, one cannot predict which patients on a therapeutic regimen will exhibit side effects or at what dose.

Some medications are commonly used to abort an active seizure or to interrupt a seizure flurry. These include diazepam (Valium) and lorazepam (Ativan). Drugs used only in the treatment of refractory status epilepticus include paraldehyde (Paral), midazolam (Versed), and pentobarbital (Nembutal).

Bromides, the first of the effective anticonvulsant pure compounds, are no longer used in humans due to their toxicity and low efficacy.

Palliative surgery for epilepsy is intended to reduce seizure frequency or severity. For example, a callosotomy or commissurotomy is performed to prevent seizures from generalizing, i.e., from being transmitted to the entire brain, which results in loss of consciousness

Vagus nerve stimulation (VNS) controls seizures with an implanted electrical device, similar in size, shape, and implant location to a pacemaker. The implanted VNS device connects to the vagus nerve in the neck and is set to emit electronic pulses to stimulate the vagus nerve at pre-set intervals and milliamp levels. About 50% of individuals with an implanted VNS device showed significantly reduced seizure frequency.

The Responsive Neurostimulator System (tRNS), in clinical study prior to regulatory approval, is a device implanted under the scalp with leads implanted either on the brain surface or in the brain close to the area where the seizures are believed to start. At the outset of a seizure, small amounts of electrical stimulation are delivered to the brain to suppress the seizure. The RNS system differs from the VNS: the RNS system is patient responsive in that it directly stimulates the brain, whereas the VNS system provides physician-determined pre-set pulses at predetermined intervals. The RNS system is designed to respond to detected signs that a seizure is about to begin and can record events and allow customized response patterns that may provide a greater degree of seizure control.

One class of therapeutic agents for treating epilepsy are the carbonic anhydrase inhibitors, but all have undesirable side effects.

Acetazolamide (Acz), a known inhibitor of carbonic anhydrase, is one such agent. It prevents hypoxic pulmonary vasoconstriction (HPV) and thus is also used to treat altitude sickness, glaucoma, and benign intracranial hypertension. Acetazolamide, however, affects kidney function because it reduces NaCl and bicarbonate reabsorption in the kidney proximal tubule. The reduction results in a mild diuretic effect, although it is partially compensated by the kidney distal segment and the metabolic acidosis produced by the bicarbonaturia. Methazolamide, also a carbonic anhydrase inhibitor, is longer-acting than acetazolamide with fewer kidney effects. Dorzolamide, a sulfonamide and topical carbonic anhydrase II inhibitor, reduces the elevated intraocular pressure in patients with open-angle glaucoma or ocular hypertension that are insufficiently responsive to beta-blockers. Inhibition of carbonic anhydrase II in the ciliary processes of the eye decreases aqueous humor secretion, presumably by slowing the formation of bicarbonate ions with subsequent reduction in sodium and fluid transport. Topiramate is a weak inhibitor of carbonic anhydrase, particularly subtypes II and IV. It is a sulfamate-substituted monosaccharide that is related to fructose. In is approved in the U.S. as an anticonvulsant to treat epilepsy, migraine headaches, and Lennox-Gastaut syndrome. Its inhibition of carbonic anhydrase may be sufficiently strong to result in clinically significant metabolic acidosis.

Acetazolamide and other calcium-inhibiting sulfonamides increase intracellular pH and relax mesenteric arteries preconstricted with norepinephrine. Calcium inhibitors and/or the intracellular alkalinization activate a calcium-dependent potassium channel, resulting in hyperpolarization of the vascular smooth muscle cell, reduction of voltage-dependent calcium channel activity, decreased intracellular calcium, and vasorelaxation.

Spreading depression (SD) is a pathophysiologic event characterized by depressed EEG activity and a change of the direct current potential as an indicator of a short-lasting cell membrane depolarization. It may be induced by a variety of cortical stimuli, including potassium chloride or glutamate application, and electrical or mechanical stimulation; it also occurs secondary to ischemia. It is accompanied by severe changes in ion homeostasis and water shifts from the extracellular to intracellular space, mirrored by changes of electrical impedance and direct current (DC) potential. The area of depolarization spreads along cortical tissue like a wave, moving away from the initiation site toward the periphery, and propagates with an estimated velocity of 3 mm/min. Electrical measurements from the cortex surface show negative deflection of the DC potential, lasting 1 to 2 minutes, combined with EEG suppression. Under normoxic conditions, SD is not followed by permanent neuronal damage, and the depressed neuronal activity is compensated by increased glucose metabolism and blood flow during the repolarization phase. The cell membrane repolarization requires an enormous metabolic effort and is compensated by increased glucose metabolism and increased blood flow.

Serotonin homeostasis, regulated by serotonin receptor 1A (Htr1a), is required for normal serotonin levels. Htr1a also mediates autoinhibition of serotonin production; excessive serotonin autoinhibition is associated with sporadic autonomic dysregulation and death. Tryptophan, a serotonin precursor, increases serotonin production. Administration of the selective Htr 1a antagonist WAY100635 completely shuts down serotonin-induced neuron impulses, resulting in apnea preceded by bradycardia; both lung function and heart function were affected.

Spreading depression (SD) has been extensively studied and is likely an important mechanism in several human diseases. Cerebral hemodynamics, i.e., cerebral blood volume and water changes, were assessed by high-speed MRI during potassium-induced spreading depression. MRI images, and brain voltage readings, were used to determine apparent diffusion coefficients over time that correlated with potassium flux along the cortex. Acetazolamide treatment resulted in vasodilation and arrested spreading depression.

Diffusion-weighted imaging is highly sensitive to slowing water proton translations early in the ischemic episode, i.e., within minutes. MR imaging measured the ADC of brain water decreases by 30% to 60%, and recent findings suggested significant apparent diffusion slowing (ADC decreases) in stroke results predominantly due to cellular swelling and reflects a shift of relatively faster translating extracellular water protons into a more hindered intracellular environment. It has been shown that when the Na+/K+ pump is disabled by intraparenchymal ouabain, the ADC decreases. This supports a link between altered ion homeostasis and alteration in ADC. There is a relation between membrane polarization and diffusion as measured by the ADC. Failure of the transmembrane ion pumps and subsequent loss in cell membrane potential is immediately followed by disruption of ion homeostasis. The resulting ionic imbalance causes an osmotically driven flow of water into the cells. MR imaging indicates the subsequent cell swelling with restricted extracellular or intracellular diffusion, and increased extracellular tortuosity, reduces the ADC.

The concept of cell preservation by quantum dot administration and treatment applies to the above these diseases and reduces degeneration of all brain cells (nerve cells, glial cells, etc.).

Particles are useful in providing repeated electric pulses either to the brain, spinal cord, or isolated nerve cells that are involved with various neural disorders. In disorders involving these regions low level brain, spinal cord, etc. neural pulses are not passing through for one reason or another, e.g., synapses, scar, misdirection, etc., and are released either as a giant pulse or can circuit back and forth until the membrane potential is completely exhausted. Therefore a pulsed stimulation by an external source, such as light or electric pulses applied to the brain, ventricles, spinal cord, cerebrospinal fluid, having quantum dots and/or semiconducting nanowires would eliminate an avalanche of the pulses in posttraumatic epilepsy, restless leg syndrome, spinal cord epilepsy, etc. A version of this concept could be potentially used to modify brain waves needed for sound sleep, alleviation of depression, etc. Stimulation of the olfactory nerve can enhance neuronal regeneration in the brain in aging adults or in Alzheimer's disease or slow its progression.

In one embodiment the method includes tunability or adjustment of duration and repetition rate or frequency of stimulation in response to cell activity. For example, saccadic eye movements are generated by underlying activity in the cortical cells of the brain, and tend to reflect a summation of the polarization and depolarization of brain cells during diurnal activity and sleep. These depolarization/repolarization or “pulse” frequencies may be influenced by various physiological and, potentially, pathological processes in the brain, monitored to diagnose abnormal patterns in the underlying activity, and altered by therapeutic stimulation of the particles to counteract abnormal activity. Under normal conditions, intrinsic electrical stimulation of the frontal eye fields elicits voluntary or so-called pursuit eye movements, but includes saccadic movements having a frequency of about 27 Hz to 36 Hz during diurnal activity, and up to about 40 Hz to 45 Hz during the rapid eye movement (REM) stage of sleep, Rio-Portilla et al., Int' J. Bioelectromagnetism 10(4) (2008), pp. 192-208. Under abnormal conditions such as epilepsy, etc., pulse avalanches in the brain can effect these saccadic movement frequencies and produce abnormal movement reflecting the underling abnormal condition. Saccadic movement frequencies may range from about 1 Hz to 1000 Hz. A frequency below 20 Hz or above 60 Hz may indicate an abnormality.

In one embodiment the pulse frequency of brain neuronal activity is evaluated using the observed frequency of saccadic eye movements. The observed frequency may be measured using known eye tracking units during diurnal activity and/or an electro-oculogram during both diurnal activities and sleep, i.e., when the eye is potentially closed. The evaluated condition may be used to determine when therapeutic light pulses are to be delivered to particles administered to the eyes, the brain, etc. In one embodiment the particles are conjugated with membrane ion channel activators, as described above.

In one embodiment an eye tracker is used in combination with a light source to therapeutically stimulate particles provided to the eye. A small digital camera may be mounted on the patient's head, e.g., in eyeglasses, to obtain video images of the eye and transmit the images to a computer. The video images may include reflected infrared, visible, and/or ultraviolet light reflected from the eyes and captured by the camera. The video images may be analyzed to determine the average frequency of saccadic movement of the eye for an interval of time, and to compare the average frequency to one or more criteria for apparently normal or abnormal brain function. The light source, e.g., LED or low powered laser, may be activated to stimulate the particles administered to the brain or inhibit an action potential response in the brain at a predetermined frequency using physician-determined pulses of light for predetermined durations at predetermined repetition intervals. The light source in one embodiment emits light that is reflected into the eye through a stationary or rotating mirror positioned within the visual field of the eye. This system is designed to respond to detected signs that a seizure is about to begin, permitting customized response patterns that may provide a degree of seizure control.

In one embodiment equipment similar to that previously described may be used to provide enhanced vision to a patient, e.g., a patient having damaged or diseased outer photoreceptor segments. A small digital camera may be mounted on the patient's head, e.g., in eyeglasses, to obtain video images. In this embodiment, however, the video images are obtained from the viewpoint and across the visual field of the patient, i.e., are images of the external environment, rather than of the eye itself. The images may approximate those viewable using only visible light or be hyperspectral images including infrared, visible, and/or ultraviolet wavelengths. The light source, emitting at least one wavelength of light, may be activated to stimulate the particles administered to the eye in a pattern representative of the video image. For example, color images are typically represented as a combination of images in three primary colors, but may be converted to a combination of images in only two colors or a single image varying only in relative intensity. Particles adapted to specifically bind to one or more of the S-cone, M-cone, and L-cone photoreceptor cells may be activated by pulses of different wavelengths to stimulate the perception of colors. Particles adapted to bind to photoreceptor cells generally, rods, or alternate targets in signaling pathway such as photoreceptor cell body, bipolar ganglion cells, amacrine cells, and Muller cells, may be activated by pulses to stimulate the perception of intensity, i.e., to simulate vision under low-light conditions. In one embodiment, placement of the photovoltaic particles in the membrane mimics the naturally-occurring amphiphilic transmembrane proteins, which have hydrophobic membrane-spanning domain(s) that interact with fatty acyl groups of the membrane phospholipids and hydrophilic domains extending into the aqueous medium on each side of the membrane. An embedded nanoparticle, e.g. with the metal portion inside the cell, acts as a photovoltaic cell where the electric current varies with the rate of photon absorption. Illumination of embedded particles generates a photovoltage that reduces the potential across the cell membrane by about 10 mV. Such membrane depolarization causes enough voltage-sensitive Na+ ion channels to open to generate an action potential that travels down the axon.

The stimulated photoreceptors will transmit the stimulated pulses to the optic nerve and to the brain, where the pulses will be interpreted as images by the visual cortex. The light source may be a complex source, e.g. a small scale LCD or OLED screen positioned in front of the eye, e.g. as a lens of glasses, or to reflect from a stationary mirror positioned within the visual field of the eye. The light source may alternately be single or multiple wavelength scanned-beam system, using one or more discrete light sources, e.g., LEDs or low power lasers, and a rotating mirror to stimulate, pixel by pixel, the photoreceptor cells, the outer segment of the retina, the inner segment of the retina, etc., similar to the manner in which an electron gun excites the phosphors of a cathode ray tube television. The computer may manipulate the image size, intensity, contrast, etc. to improve visibility, as well as to translate between detected wavelengths of light, e.g., the typical red, green, and blue color-filtered detectors employed in Bayer filtered sensors or multi-sensor imaging blocks, and emitted frequencies of light emitted at the appropriate wavelengths to stimulate the one or more types of particles. The particles in the retina can respond to both detection of IR light that is reflected from a real object that acts on the particles, or detection of IR light that is captured by a digital camera and is reemitted by a head-mounted device, with the camera and processor able to amplify the pulse frequency, energy, etc.

In one embodiment an eye tracker is used in combination with a light source to therapeutically stimulate particles provided to the brain. For example, a controller may analyze output from pairs of electrodes placed around an eye to determine the average frequency of saccadic movement of the eye for an interval of time, and to compare the average frequency to one or more criteria for apparently normal or abnormal brain function. Particles administered to the brain, and illuminated by the light source through a window in the skull, an implanted light guide, a fiber optic material, etc., or alternatively using an LED implanted under the skull that is remotely activated to produce the light source, may be stimulated at a predetermined frequency using physician-determined pre-set pulses of light at predetermined intervals. The predetermined frequency and predetermined intervals may be selected to simulate normal electrical activity of the brain to prevent or dampen the effect of abnormal activity generated in, e.g., an epileptic seizure, etc. Alternatively a wavelength can be used that suppresses the activity of those neurons and blocks the acute process for the desired time, and then can one start the process with a normal frequency of stimulation. This embodiment may be used to modify the electrical pulses and involuntary movements in Parkinson's disease.

In one embodiment a controller is combined with a light source and a window in the skull, an implanted light guide, a fiber optic device, etc., to create a form of pacemaker that may be externally controlled. In one embodiment, by therapeutically stimulating the brain at pulse frequencies such as those found in REM sleep, the device may help the patient to achieve sleep or diminish a disturbed mental state such as depression, aggression, psychosis, etc. The system may be adapted to be remotely controlled by a physician or medical staff and include a wireless receiver or transceiver. Such a system may be fully implantable or have an external controller and battery unit. The system may also be adapted to be controlled by the patient, and may include a governing system limiting the frequency and/or duration of self-activation.

In one embodiment such a stimulation system is adapted for use as a pacemaker for the heart, controlling the frequency of activation of the sinoatrial node and/or atrioventricular node to control cardiac contractions. For example, particles conjugated with membrane ion channel activators may be coated on or included in fiber optics implanted within the right ventricle.

A physician may select specific properties and emission frequencies to selectively regulate polarization in specific sites and for specific results. Thus, the particles are tunable to provide desired properties; for example, they may be size specific, current specific, patient specific, disease specific, activation specific, site specific, etc.

As one example, particles provided throughout the retinal layers may be selectively regulated to normalize polarization and/or reduce or prevent repolarization, depolarization, and/or hyperpolarization. As another example, selected particles may be administered to selected sites and selectively regulated (e.g., temporally, spatially, activationally, etc.) to result in different effects to fine-tune a desired outcome. More specifically, a patient's progress may be monitored after a slight regulation and, if warranted, further regulation may be administered until a desired outcome is obtained. For example, a patient with muscle tremors may be treated with the inventive method for a duration, extent, activation energy, etc. to selectively repolarize striated muscle cells until a desired effect is reached.

In one embodiment a patient with cardiac disease or dysrhythmia, including cardiac arrhythmia, is treated with a biocompatible quantum-dot/gene conjugate coated or otherwise containing anti-cardiac muscle antibodies. The quantum dots are administered by intravenous or intracardial routes, e.g., during a cardiac catheterization procedure. Once administered, cardiac cells are then be stimulated with, e.g., an implanted fiber optic device connected to a control system and light generator to stimulate or regulate the cardiac rate as needed. The fiber optic device and its controller are implanted under the skin of the chest, and function similarly to a cardiac pacemaker. In one embodiment, the device and its controller are programmed to automatically initiate so that a pulse is obtained upon cardiac arrest. This embodiment eliminates need for an external defibrillator, which provides indiscriminate electrical action and thus is traumatic.

In embodiments, the disclosed complex comprises nanoparticles other than quantum dots; these include nanowires, nanorods, etc. In embodiments containing a biomolecule, the complex comprises at least a first nanoparticle and a second nanoparticle where the first and the second nanoparticles absorbs energy at different wavelengths, and thus are activated by different energy wavelengths, e.g., light. This embodiment permits control of the activity of the complex, e.g., selective activation using different energy wavelengths, providing further control of the physiological function of excitable cells. In embodiments comprising a biomolecule that targets the complex to a specific location, tissue, cell, etc., the complex comprising multiply excited particles can be used for diagnostic identification. For example, it can be used to identify a specific cell type.

In one embodiment, the particles are mixed into or with a biocompatible fluid that may include one or more types of indirectly associated (non-conjugated) biomolecule. In another embodiment, the particles are in the form of beads or spheres. In another embodiment, the particles are provided as a film. In another embodiment, the particles are drawn and provided as fibers. In any of these embodiments, the particles are provided to a patient by injection to other minimally invasive techniques known to one skilled in the art.

Upon administration, the particles are disseminated and/or located intracellularly (within a cell), intercellularly (between cells), or both intracellularly and intercellularly. They may be administered in a number of ways. With respect to the eye, they may be injected through the retina, under the retina superiorly, over the fovea, through the outer plexiform layer down to the fovea, into the vitreous cavity to diffuse through the retina, etc. The procedure permits particles to be located at any site including the macula, that is, the particles may be directly on the macula, directly on the fovea, etc. distinguishing from procedures requiring electrodes to be located beyond the macula or beyond the fovea so as not to block foveal perfusion. The procedure does not require major invasive surgery and is only minimally invasive, in contrast to procedures that involve surgical implantation of an electrode or photovoltaic apparatus. The procedure locates particles diffusively substantially throughout the eye, or selected regions of the eye, in contrast to procedures in which an electrode or other device is located at a single site. Thus, the site of treatment is expanded with the inventive method. In this way, the particles locate within excitable cells, such as the retina, macula, etc. using an ocular example, and also locate between these excitable cells, and are thus dispersed substantially throughout a region of interest. Particles not located as described are handled by the retinal pigment epithelium.

In one embodiment, and as an example, stem cells are grown or incubated in the presence of antibody and gene-coated magnetic particles, e.g., quantum dots, to permit their digestion of quantum dots or attachment of the quantum dots to the cells. In one embodiment, and as an example, stem cells are grown or incubated in the presence of antibody and channel protein gene coated magnetic particles, e.g., quantum dots, to permit their digestion of quantum dots or attachment of the quantum dots to the cells. After administration of stem cells and quantum dots in the desired area or in the circulation, a fiber optic light and a magnet are placed at the intended area to attract and guide the magnetic quantum dots to that area and enhance tissue repair.

In one embodiment the stem cells and quantum dots are injected in the vitreous cavity, in or under the retina, combined with placement of the magnet over or near the retina on the back of the eye. This embodiment directs the stem cells and quantum dots to the specific areas of the retina, optic nerve, etc. and enhance tissue repair.

In one embodiment the stem cells and quantum dots are injected in the cerebrospinal fluid, brain, spinal cord, or tissue near a peripheral nerve. A light and a magnet are placed in or near the damaged areas to direct the stem cells and quantum dots to the degenerative areas of the brain, spinal cord, or peripheral nerve and enhance tissue repair.

In one embodiment the stem cells and quantum dots are injected in the circulation as needed and are captured with an external magnet placed in a desired area.

In each of these embodiments and example, the stem cells and quantum dots can be stimulated as described with light to enhance tissue repair.

Continuing to use the eye as a non-limiting example, the particles migrate through spaces of retinal cells and distribute through retinal layers, including the RPE. To even more widely disperse particles throughout the retina, they may be sprayed over the retina. In one embodiment, they may be delivered and distributed throughout the retinal layers by a spraying or jetting technique. In this technique, a pressurized fluid (liquid and/or gas) stream is directed toward a targeted body tissue or site, such as retinal tissue, with sufficient energy such that the fluid stream is capable of penetrating the tissue, e.g., the various retinal layers. In applications, the fluid stream, which may be a biologically compatible gas or liquid, acts as a carrier for the particles. By way of example, the spraying technique has been used in cardiac and intravascular applications for affecting localized drug delivery. The teaching of those applications may be applied to the delivery of the particles to the retina. For example, U.S. Pat. No. 6,641,553 which is expressly incorporated by reference herein, discloses pressurizing a fluid carrier having a drug or agent mixed therewith and jetting the mixture into a target tissue.

It will also be appreciated that other agents may be included in the fluid in addition to the particles. These other agents include, but are not limited to, various molecules, drugs that have stimulatory or inhibitory activity (e.g., protein drugs, antibodies, antibiotics, anti-angiogenic agents, anti-prostaglandins, anti-neoplastic agents, etc.), vectors such as plasmids, viruses, etc. containing genes, oligonucleotides, small interfering RNA (siRNA), microRNA (miRNA), etc.

In one embodiment, quantum dots conjugated or otherwise associated with a molecule or biomolecule are delivered to an eye to enhance functional recovery of an at least partially functional retinal cell in a patient in need of such treatment. This embodiment of the method may be in addition to, or in place of, the method of regulating membrane polarity using the introduced quantum dot previously described. The quantum dot-biomolecule conjugate or particle may be provided to a retinal cell cytoplasm or a retinal cell nucleus, with injection or other introduction means into the subretinal space, into the retina itself, into the macula, under the macula, into the vitreous cavity with vitreous fluid present, and/or into the vitreous cavity with vitreous fluid absent. The quantum dots conjugated or otherwise associated with a vector carrying a protein or other molecule capable of modifying genes in retinal cell provides gene therapy. In one embodiment, racking means (e.g., sensors or other signals) associated with the complex are used to monitor location, stability, functionality, etc. of the complex.

In one embodiment the retinal or other cell so modified by the method contains a light-sensitive protein that itself may be excited directly by light of a specific wavelength, or in an alternative embodiment, be excited by light of a different wavelength or produced by the quantum dot (e.g., fluorescence) after the quantum dot is excited upon exposure of light. For example, if the modified genes of the cell produce halorrhodopson, then the quantum dots to which the halorhodopsin-encoding gene were associated can be excited to then activate the halorhodopsin to silence the cell. If the modified genes of the cell produce channelrhodopsin, then the quantum dots to which the channelrhodopsin-encoding genes were associated can enhance an action potential. As known to one skilled in the art, channelrhodopsins, a family of proteins, function as light-gated ion channels in controlling electrical excitability among other functions. As known to one skilled in the art, halorhodopsin is a light-activated chloride-specific ion pump. When quantum dots are combined with channelrhodopsins or halorrhodopsons, quantum dots enhance the effects of these proteins, and result in enhanced cell polarization responsive to light stimulation.

In one embodiment, quantum dots conjugated or otherwise associated with a molecule or biomolecule are delivered to the heart to enhance functional recovery of an at least partially functional heart cell in a patient in need of such treatment.

In one embodiment of monitoring, a video camera receives an image of the external environment that is projected into an eye containing the functional, excitable retinal cell to be treated. For example, after initial administration of the quantum dots to the eye, a camera mounted on or in the eyeglasses records and produces a digitized image of the external environment, which is then transmitted to a small computer mounted on the glasses. The picture can be recreated on an LCD using a diode array. This image, in turn, is projected through the pupil, onto the retina containing quantum dots to stimulate rods and cones. This process may be optionally repeated to determine the extent or degree to excite the quantum dots and/or to achieve the desired cell polarization state by evaluating retinal function, e.g., by electroretinogram or other methods known to one skilled in the art.

In one embodiment, the eye imaging method, e.g., OCT, confocal microscopy, provides a method of tracking the quantum dots in cells, e.g., stable cells such as neurons.

In one embodiment, the treated cells are restored to normal polarization by treatment using the quantum dots; and concomitantly, the cells are treated with a biological moiety conjugated to the quantum dots to relieve, restore, ameliorate, or treat a functional condition of the retinal cell, e.g., a retinal genetic disease. In one embodiment, the biologically active conjugate is biologically active after the quantum dot ceases to be functional. In one embodiment the quantum dot is active after the biologically active conjugate ceases to be functional.

As schematically shown in FIGS. 9 and 10, a device 150 for delivering the particles to the retina generally includes an elongated tube or cannula 152 having a proximal end 154 and a distal end 156 and an interior lumen 158 extending between the proximal and distal ends 154, 156. A distal end region 160, which may include a distal end face or a portion of the outer surface of the cannula 152 adjacent the distal end 156, includes a plurality of outlet ports or apertures 162 in fluid communication with the interior lumen 158. The device 150 further includes a pressure control source 164, such as for example a fan or pump, in fluid communication with the lumen 158 and operable for establishing an elevated pressure within the lumen. As known to one skilled in the art, the pressure should be sufficient to effectively disseminate the particles throughout the retina through a spraying or jetting action, but not sufficient to substantially damage retinal tissue. In one embodiment, a pressure may range from 0.0001 psi to 100 psi. The pressurized spraying also assists in distributing particles that disseminate and localize throughout the retinal layers. Localization of the particles permits enhanced control, duration, ease, etc. of stimulating these particles, resulting in enhanced control and effect.

The particles are introduced into the interior lumen 158 from any source, such as from a reservoir chamber, a syringe, etc. (not shown), and are mixed with a carrier fluid 166 such as a biocompatible gas or liquid. As non-limiting examples, air, oxygen, nitrogen, sulfur hexafluoride other perfluorocarbon fluids, etc., alone or in combination, may be used.

The pressurized fluid carrying the particles is regulated for ejection from the outlet ports, and is propelled toward the retina. The diameter of the outlet ports and pressure of the fluid are such as to allow the particles to penetrate the retinal tissue with minimal or no retinal damage. To accomplish a wide distribution of the particles throughout the retinal layers, the pressure may be pulsed to vary the penetration depth of the particles. The cannula may also be rotated or moved to spray or cover a larger area of the retina. Those of ordinary skill in the art will recognize other ways to distribute the particles throughout the retinal layers. As one example, the diameter of the outlet ports may be varied to provide different penetration depths. The outlet port diameters may range from about 0.01 mm to about 1 mm. As another example, the angles of the outlet ports may be varied to provide different spray patterns.

The above-described device may be used in the inventive method to deliver particles to the retina and distribute them substantially throughout the retinal layers, both intracellularly and/or intercellularly. That is, the particles diffusively locate and penetrate the retinal layers.

In one embodiment, an ocular surgeon may remove the vitreous gel, such as by an aspiration probe having vacuum pressure or a cutting probe, and replacing the contents of the vitreous cavity with saline, air, or another biocompatible fluid to facilitate particle penetration. The spraying device is inserted through the incision and into the vitreous cavity. The distal end of the device is positioned on or adjacent the retina, with the surgeon verifying placement using an operating microscope, a slit lamp, or other methods known in the art. Once the distal end of the device is adequately positioned, the pressurized fluid stream carrying the particles is generated and the particles are propelled toward the retina so as to distribute the particles throughout the retinal layers, as previously described. A gas probe may also be inserted into the vitreous cavity, such as by a second incision, to maintain the desired intraocular pressure. In another embodiment, the vitreous gel is not removed and the particles are injected (e.g., using a needle or other type of injection device) without spraying close to the retina, where the particles then diffuse through intercellular spaces of the retina and throughout the eye. Those of ordinary skill in the art will recognize that while the delivery method has been described as using separate aspiration probes, fiber optic probes, and gas probes, a single device that accomplishes delivery of the particles to the retina, removal of the vitreous gel and gas delivery may be used in the inventive method.

Once located at the desired location, the particles are stimulated using an energy source. The energy source may be located external to the eye at either or both the front and back, external to the retina, or on the surface of the retina. Because the retina is transparent, light is able to pass through and hence activate the particles located on and in various retinal tissues, as is subsequently described. The activated particles reset or influence the plasma membrane electrical potential of excitable cells, resulting in a desired response in membrane polarity. As previously described, this may take the form of normalized polarization, repolarization, enhanced polarization (i.e., stimulation), or reduced polarization (i.e., calming), etc.

In one embodiment, the particles are delivered into the eye when the vitreous gel is removed and replaced with saline and the internal limiting membrane (ILM) is removed. In one embodiment, the internal limiting membrane is removed to permit particle dissemination within the retina and throughout retinal intracellular spaces. This enhances diffusion of particles in the retina so that, by fluid flow, particles can then disseminate and penetrate retinal layers. Particles may adhere to the outer cellular membrane and/or may enter retinal cells. The particle size and/or spraying pressure, location, formulation may be altered to aid in selectivity. Particle penetration may be limited by the external limiting membrane (ELM), which may act as a semi-barrier to retinal transport. Excess particles may be removed as a part of the normal phagocytosis process (e.g., by glial cells). Ganglial cells in the eye, responsible for visual processing (discerning motion, depth, fine shapes, textures, colors), have less active phagocytosis mechanisms, so treatment of these cells may be affected by spraying to minimize excess distribution of particles.

Repolarization of cell membranes in a first location may have beneficial effects on polarization of cell membranes in second and subsequent locations. Due to propagation of electrical stimuli, a wave of electrical distribution is disseminated throughout the retina, for example, along a glial cell network. Because the glial cells assist in maintaining electrical balance, propagation also stabilizes polarization of adjacent cells.

It will be appreciated from the above description that stimulation of the entire retina may be achieved, rather than stimulation of a portion of the retina in proximity to a fixed electrode. This achieves substantially uniform repolarization, minimizing or preventing areas of hyper- and/or hypo-polarization, which assist in functional regeneration of glial cells.

In one embodiment, an ocular surgeon may stimulate the particles with an external light source, by ambient light, by ultrasound radiation, or by other mechanisms known to one skilled in the art. The particles facilitate, enhance, or boost a biological cell's regulation of its polarity, with adjacent cells capable of being stimulated due to the glial stimulus-propagating network.

It will be appreciated that nanoparticles are not limited to the previously described quantum dots and nanotubes, and include other carbon-based skeletal-type structures. Examples of such structures encompassed by nanoparticles and/or nanostructures include, but are not limited to, fullerenes, bucky balls, micelle-like structures such as micellar nanoparticles (MNP), lipid-based liposomes, dendrimers, and single-stranded deoxyribonucleic acid- or ribonucleic acid-oligonucleotide aptamer-conjugated or modified nanoparticles.

In one embodiment of the inventive method, such nanoparticles function as carriers and deliver a variety of opsin gene families. Opsin family genes include rhodopsin, halorhodopsin, photopsin, and channelrhodopsin. As known in the art, such gene families can integrate into the cell nucleus and nucleolus. The opsin gene families can be delivered alone, or in combination with one or more other genes to correct a genetic defect of excitable cells, or to induce an action potential in a membrane of excitable cells. Excitable cells include retinal cells, cells of the central nervous system (CNS) or peripheral nervous system (PNS). Stimulation may be directly or indirect, and may be by an external light source or a fiber optic. The method may also be used to stimulate cells that are normally non-excitable, e.g., stem cells including pluripotent mesenchymal stem cells, fibroblasts, glial cells, etc. Other desired gene stimulators, e.g., promotors, or gene silencers, e.g., siRNA, may also be included to up- or down-regulate gene function and enhance tissue repair.

In this embodiment, the nanoparticles are rendered biocompatible, or their biocompatibility is enhanced, by being coated or associated with biocompatible molecules. As known in the art, these biocompatible molecules include, e.g., biotin-streptavadin, (poly)ethylene glycol (PEG), acetyl cysteine, cell penetrating peptide (CPP), arginine-CPP, cysteine-CPP, etc.

In one embodiment, the method is performed on a patient having a condition where excitable cells are partially or completely lacking function, and the method provides delivery of rhodopsin or other opsin-family gene members and stimulates excitable cells. In one embodiment, the method is performed on a patient where it would be beneficial to stimulate cells that are normally non-excitable, and the method provides delivery of rhodopsin or other opsin-family gene members and stimulates such cells that are normally non-excitable.

In one embodiment, the method is performed on a patient having a condition involving a defective gene, and where the condition is in a relatively early stage, amenable to therapy by the inventive method. The method results in stimulation of cells and prolongation of a regenerative process. The method may permanently repair the condition when the defective gene is included with the nanoparticle containing the opsin-family gene.

The nanoparticle-gene(s) composition may be administered intraocularly by intravitreal, intraretinal, subretinal, or other site of injection. The nanoparticle-gene(s) composition may be administered intrathecially into the cerebrospinal fluid, brain, or spinal cord. Intrathecal injection protects the gene(s) that otherwise may be damaged by contact with fluid and/or cellular blood components. The nanoparticle-gene(s) composition may be injected in any location, e.g., heart, peripheral nerves, etc., with a small gauge insulated metallic needle connected to a battery to carry electricity in the tissue for electroporation delivery inside the desired cells. Other types of force may also be simultaneously applied to enhance cell penetration of the nanoparticle. These embodiments facilitate penetration of the nanoparticle-gene(s) complex into cells. Subsequent stimulation of the cells is achieved using a fiber optic to stimulate nerves, cardiac muscle, skeletal muscle, etc. replacing electrical stimulation with light stimulation using a diode laser.

The nanoparticles may further be encapsulated to protect their contents. As only one non-limiting example, the dendrimer poly(amidoamine) (PAMAM) can be functionalized to be biocompatible and cell penetrating. Other dendrimers are poly(amidoamine-organosilicon) (PAMAMOS), poly(propyleneimine) (PPIO), tecto, multilingual, chiral, hybrid, amphiphilic, micellar, multiple antipen peptide, and Frechet-type dendrimers. Dendrimers have been proposed as carriers of luciferase gene, but there are no reports using or suggesting their use to transfer opsin gene family members, activated by light and producing an action potential on cells. The nanoparticles may be rendered visible, e.g., by combining them with other nanostructures such as functionalized quantum dots.

Fullerenes and buckyballs are nano-sized three dimensional carbon molecules having hollow spherical, oval, or tubular structures. Dendrimers are highly symmetrical nano-sized spherical compounds composed of branched polymers that have many functional groups on their outer surface. They are made with variable functionality, thermal stability or solubility, and are commercially available or synthesized as known in the art.

The nanoparticles have a small size of up to about 1 nm. The combined nanoparticle-gene(s) complex may likewise have a small size or up to 3 nm to 800 nm or more, as long as they are able to pass through intracellular spaces of the retina or CNS.

In one embodiment, administration of the inventive method into stem cells causes cellular multiplication upon light stimulation, with the stem cells becoming activated after light pulse exposure. Stem cells thusly treated migrate to the light source where they grow and replace cellular loss in a tissue and enhance tissue repair. This embodiment could be used for therapy in pathologies such as, e.g., age related macular degeneration, and in organs such as heart, spinal cord, or brain. Light is delivered to the desired site using a fiber optic. In this way, damage due to stroke, infarct, is repaired.

In one embodiment, nanoparticles include all particles having a size ranging from of <1 nm to <1 micron. These include, but may not be limited to, quantum dots, dendrimers, fullerenes (buckyballs), liposomes, microspheres, lipids, and/or combinations thereof.

The nanoparticles may be synthetic, organic (e.g., liposomes), non-organic, non-magnetic, magnetic, paramagnetic, diamagnetic, supramagnetic, non-magnetic, mesoporous carbide-derived carbon, iron oxide nanoparticles with gold, graphene oxide and mesoporous silicone nanostructures, carbon, quantum dots, nanoshells, nanorods, nanotubes, nanowires, quantum dots, etc. Illustrative and non-limiting specific examples also include liposomal nanoparticles, liposome-PEG nanoparticles, micellar polymeric platform nanoparticles, L-adenine nanoparticles, L-lysine nanoparticles, PEG-deaminase nanoparticles, polycyclodextrin nanoparticles, polyglutamate nanoparticles, calcium phosphate nanoparticles, antibody-enzyme conjugated nanoparticles, polymeric lipid hybrid nanoparticles, nanoparticles containing a combination of two-three elements such as gold, gold-iron oxide, iron-zinc oxide, metallic nanoparticles, polylacticglycolic acid nanoparticles, ceramic nanoparticles, silica nanoparticles, silica crosslinked block polymer micelles, albumin-based nanoparticles, albumin-PEG nanoparticles, dendrimer attached magnetic or non-magnetic nanoparticles, etc.

In one embodiment, the nanoparticles may be incorporated within liposomes and/or plasmids carrying DNA, RNA, siRNA, medications, etc.

In one embodiment, the nanoparticles may of any shape, e.g., spheres, nanotubes, nanowires, tetragons, hexagons, cylinders, etc.

In one embodiment, the nanoparticles are coated with PEG, PEI, chitosan, biotin, streptavidin, CPP, ACPP etc. along with the specific cell membrane antibodies that can be tested by enzyme linked immunoassay (ELISA) to attach to the cell receptors.

In one emboidiment using nanoparticles for gene transfer, a plasmid containing a gene (e.g., rhodopsin, holorodopsin, etc.) is attached to the nanoparticles during the coating process with PEG, chitosan, etc. along with CPP or ACPP. Nanoparticles generally may have improved tolerance in vivo compared to quantum dots. In this embodiment, use of magnetic nanoparticles is desirable for gene transfer because a magnetic field increases the transfection in tissue. In this embodiment, magnetic nanoparticles are administered along with a localized magnet. The electrostatic potential of these nanoparticles can be as high as −25 mV, encouraging further cell membrane penetration. While magnetic nanoparticles may be desirable for some embodiments, non-magnetic nanoparticles and organic nanoparticles can also be used with plasmids for transfection.

In one embodiment using nanoparticles for gene transfer, a liposome containing a gene (e.g., rhodopsin, halorhodopsin, etc.) is used, forming a nanoparticle-liposome-gene complex. Liposomes as a carrier for the nanoparticle and gene are then coated and conjugated with the antibody prior to administration to the patient. Methods of preparing liposomes are known in the art, e.g., Akbarzadeh at al., Nanoscale Res. Lett. 8 (2013) 102, which is expressly incorporated herein by reference in its entirety.

In one embodiment, the nanoparticles are used for the transfection of cells, employing a plasmid attached to the labeled nanoparticles/genes that are stimulated by light to affect membrane channels of the cell membrane, regulating the membrane potential of the cells or inducing an action potential or a signal that can be transmitted to another cell or neuron.

Examples include, but are not limited to, opsin family genes or similar G-proteins, a group of light sensitive membrane bound G-protein-coupled receptors converting a light pulse or other signals outside the cell to an electrochemical signal, e.g. rhodopsin, holorodpsin, etc. The opsins family consists of Go opsins and Go opsins found in vertebrates or Gq opsins, photoisomerases, and neuropsins. The plasmids are used to transfer the gene-conjugated nanoparticles inside the cell. The plasmids are conjugated or entrapped during the coating process of nanoparticles with the compounds previously described, i.e., PEG, PEI, chitosan, biotin, streptavidin, CPP, ACPP etc. along with the specific cell membrane antibodies.

In one embodiment, the plasmid nanoparticles/genes are administrated systemically or along with other stimulatory or inhibitory medications, locally, e.g. in the eye, central nervous system, peripheral nerves, heart, etc.

In one embodiment when the nanoparticles/genes are injected in the eye or in the CNS fluid naked DNA, RNA, etc. conjugates are not damaged by serine proteases during the process of administration because of the existence of the blood brain and blood ocular barrier, preventing free serum access to these organs.

In one embodiment, the plasmid nanoparticles may be administered through the nasal mucosal by spraying, drops, or injection to access the olfactory nerves. The nanoparticles are picked up by olfactory nerve cells through which they travel to brain affecting the desired brain area. Thus, brain stimulation can be performed by the patient deliberately after nasal nanoparticle administration to affect various disease such as epilepsy, mood, PTSD, depression, fright, Parkinsons disease, Alzheimers disease, and other brain degenerative diseases, or after trauma or stroke, in migraines, addiction, etc.

The plasmid nanoparticles/gene can be admitted to the tissue culture of any cell. They are picked up by the cells having membrane receptors to the nanoparticles antibodies. These cells can be in the body or in the tissue culture. Examples of such cells include, but are not limited to, neuronal, retinal, muscle, or any kind or stem cells e. g. skin, neurons, mesenchymal, fibroblast, mucosal stem cells of the eye, glial cells, endothelial cells. These stem cells can be injected in the circulation, in the CNS or its fluid, eye, under the retina, spinal cord, peripheral nerves through the skin, through the nasal mucosa, in any other organ. The transfected cells or the organ can be directly stimulated by light of any wavelength, e.g. external environment, by diode laser, ultrasound, etc. Stimulation can be performed via a processor as a light pulse (from a diode laser etc.) applied to the transfected organ e.g. brain, eye, spinal cord, peripheral nerves, the heart as a pacemaker using a fiber optic implanted in the organ, or applied externally for superficially located nerves, or the retina through the cornea or directly through the sclera or, as previously described, to the brain through the nasal mucosa, etc., and enhance tissue repair. The number of pulses or their duration can be also controlled and/or predetermined by the processor. In one embodiment, application of light pulses to the transfected cells causes them to multiply and increase in number in vivo or in vitro. The number of stem cells can be increased in cells carrying nanoparticles. The stimulating light pulse creates an electrical potential inside the cell that stimulates cell growth and multiplication of the stem cells and their growth in vivo or in vitro, as is known with electrical pulse stimulation. The result is a significant contribution to the number of stem cells needed in vivo to repair damaged tissue. Such an embodiment is useful in therapy for, e.g., vascular occlusion in the retina, spinal cord, brain, and heart leading to strokes or infarcts. Such an embodiment is also useful in therapy for various diseases in the extremities or other degenerative diseases such as Alzeheimer's disease, Parkinson's disease, traumatic brain injury, spinal cord injury, etc., and enhance tissue repair.

In one embodiment, one uses magnetic nanoparticles to increase transfection of the cells. When administered in the circulation, eye, CNS, peripheral nerves, heart, etc., a magnet is applied to generate a magnetic field and attract the nanoparticles. The magnet can be a natural magnet or an electromagnet. It is positioned at the desired location of an organ where the intended cell transfection should take place e. g. over the sclera behind the retina, frontal, parietal, posterior cortex, heart, spinal cord, peripheral nerves, nose, etc. The nanoparticles accumulate over a period of time in the organ and transfect the cells. The electrostatic potential of these nanoparticles can be as high as −25 mV, encouraging further the cell membrane penetration.

In one embodiment, these plasmid nanoparticles/genes transfer the gene or genes in the predetermined cells. They are picked up by the cells having membrane receptors to the nanoparticle antibodies. After transfecting the cells, the nanoparticles can be degraded or expelled from the cells, taken up by reticuloendothelial cells, eliminated through the bile, urine, sweat, etc.

The transfected cells are stimulated by light of any wavelength from ultraviolet to infra red and other wave lengths, or by mechanical force, ultrasound, etc., to change the membrane potential of the cell and achieve normalization of the membrane potential or an action potential.

In one embodiment, biomolecules including genes are transferred to the nucleus using inorganic and/or organic solar cell nanoparticles, including hybrids, as subsequently explained, using gene editing by programmable directed nucleases to result in patient therapy. As is appreciated by those skilled in the art, therapy includes any reduction in or amelioration of pathological effects, including but not limited to curing the patient of the pathology. In this embodiment, these nucleases bind to and cut DNA that matches guide RNAs in regions containing clustered regularly interspersed palindromic repeats (CRISPR). Cas9, the CRISPR associated protein 9, is an RNA-guided DNA endonuclease associated with the CRISPR complex. The endonuclease and/or the CRISPR/Cas9 complex, upon cell entry, seek DNA containing a target sequence, e.g., a specific mutation in the mitochondria or the nucleus. The cleavage functions of the endonuclease and/or CRISRP/Cas9 complex destroy the disease-producing DNA by excising it or replacing it with an un-mutated, i.e., normal DNA segment, as known in the art to treat the patient. The nuclease cleaves host DNA at the target site. With one cut, CRISPR can insert a new customized gene sequence; with two cuts, two CRISPRs can excise the DNA, providing the host with a new DNA. The mechanism of action of the CRISPR/cas9 complex and its ability to target and edit genes is known in the art, e.g., Sander and Joung, CRISPR-Cas systems for editing, regulating and targeting genomes. Nature Biotechnology 32 (2014) 347-355 which is expressly incorporated by reference herein.

Briefly, and as previously disclosed in greater detail, inorganic solar cell type nanoparticles include the following. Graphene quantum dots, graphene-oxide quantum dots, graphene-zinc oxide quantum dots, graphene nanotubes, carbon nanotubes, and/or non-quantum dot nanoparticles; these may be non-magnetic, magnetic, paramagnetic, diamagnetic, supramagnetic, non-magnetic, mesoporous carbide-derived carbon, iron oxide nanoparticles with gold, graphene oxide, mesoporous silicone nanostructures, nanoshells, nanorods, nanotubes, nanowires, etc. Organic solar cell type nanoparticles include the following. Fullerenes, buckyballs, dendrimers, liposomes, aptamers, and/or micelles. Specific types include, e.g., dendrimers including poly(amidoamine) (PAMAM), poly(amidoamine-organosilicon) (PAMAMOS), poly(propyleneimine) (PPIO), tecto, multilingual, chiral, hybrid, amphiphilic, micellar, multiple antipen peptide, and Frechet-type dendrimers. Hybrid quantum dots include the following. Graphene/zinc oxide (ZnO) and reduced graphene oxide, plasmonic nanoparticles coated with reduced graphene oxide, and dextran-reduced graphene oxide. Exemplary non-limiting examples include liposome-PEG nanoparticles, micellar polymeric platform nanoparticles, L-adenine nanoparticles, L-lysine nanoparticles, PEG-deaminase nanoparticles, polycyclodextrin nanoparticles, polyglutamate nanoparticles, calcium phosphate nanoparticles, antibody-enzyme conjugated nanoparticles, polymeric lipid hybrid nanoparticles, nanoparticles containing a combination of two-three elements such as gold, gold-iron oxide, iron-zinc oxide, metallic nanoparticles, polylacticglycolic acid nanoparticles, ceramic nanoparticles, silica nanoparticles, silica crosslinked block polymer micelles, albumin-based nanoparticles, albumin-PEG nanoparticles, dendrimer attached magnetic or non-magnetic nanoparticles, etc.

Solar cell nano- and/or micro-particles are three dimensional semiconductor devices using light or ultrasound energy to generate electrical energy to provide a photovoltaic effect. In embodiments, the particle material may be ceramic, plastic, silicon; particles of crystalline silicon may be monocrystalline cells, poly or multicrystalline cells, ribbon silicon having a multicrystalline structure, nanocrystals of synthetic silicon, gallium/arsenide, cadmium/selenium, copper/indium/gallium/selenide, zinc sulfide, indium/gallium/phosphide, gallium arsenide, indium/gallium nitride, a nanocrystal, such as cadmium/selenium (Cd/Se) and a metal, e.g., a CdSe/Au nanometer-sized composite particle as previously described, particles of a variety of semiconductor/metal and semiconductor/semiconductor hetero-junctions, e.g., particles based upon semiconductor/metal hetero-junctions between group II-VI, IV, III-V, IV-VI, referring to groups of the periodic table, metal-oxide, or organic semiconductors and a metal, and in particular those based upon Si/Au, GaAs/Au, InAs/Au, and PbS/Au hetero-junctions. The quantum dots and/or semiconductor nanowires may also be biocompatible short peptides of naturally occurring amino acids that have the optical and electronic properties of semiconductor nano-crystals, e.g., short peptides of phenylalanine. The particles can consist of both inorganic or organic materials, as previously described.

Any of the particles may be coated with biocompatible mono- or bilayers of phospholipid a protein, a peptide polyethylene glycol (PEG) that can be used as a scaffold to aid in biocompatibility of the particle. The particles can be entirely or partially biodegradable. Particles may also be included in/coated on a bioabsorbable, or non-bioabsorbable but biocompatible, polymer fiber, tube, or other two- or three-dimensional structure. Therapeutic stimulation of the polymer and adjacent tissue may stimulate and/or inhibit cell excitation, depending upon the wavelength of the applied light and the character of the associated particles, with differing parts of the polymer, e.g., the front and back sides of a substantially two-dimensional structure, having different particles to exert different effects upon target and adjacent cells.

As previously disclosed, all the above embodiments use compounds that are collectively termed nanoparticles. Nanoplatforms may be developed, as previously described.

The nanoparticles that are coated, targeted, functionalized, and conjugated with opsin and/or other genes, replace the defective cell's gene, using enolase and/or CRISPR technology both of which are known in the art. As previously described, the nanoparticle can be functionalized to render or enhance its biocompatibility, and can be further treated or delivered to enhance cell penetration. The nanoparticles can comprise an enolase or CRISPR complex, and/or a G-protein and/or opsin-family gene, and an antibody that targets the nanoparticles to a cell and coated with a biocompatible molecule for cell uptake, forming a complex that is activated with an energy source. To enhance biocompatibility, association with or covalent coupling to one or more of cell penetrating peptides (CPP), arginine-CPP, cysteine-CPP, polyethylene glycol (PEG), biotin-streptavadin, acetyl cysteine, an antibody, and/or a ligand for a receptor may be employed. As an example, nanoparticles can be functionalized and/or linked to vectors using (poly)ethylene glycol (PEG) moieties, with the number of PEGS varied depending on various factors, such as the need to enhanced hydrophilicity, the protein size, etc. The vector and nanoparticle are combined in the presence of at least one biocompatible adjuvant, suspension agent, surfactant, etc. Particles may be coated with or linked to, e.g., folate, polydopamine, etc. so that these molecules are targeted intracellularly, extracellularly, to a cell membrane, to a specific cellular site or organelle, etc. The nanoparticles may further be encapsulated to protect their contents, e.g., the dendrimer poly(amidoamine) (PAMAM) can be functionalized to be biocompatible and cell penetrating. Other dendrimers are poly(amidoamine-organosilicon) (PAMAMOS), poly(propyleneimine) (PPIO), tecto, multilingual, chiral, hybrid, amphiphilic, micellar, multiple antipen peptide, and Frechet-type dendrimers.

In one embodiment, the nanoparticles, such as a semiconductor-metal particle, can be coated such that the nanoparticle is amphiphilic, where a portion of the nanoparticle is rendered hydrophilic and another portion of the nanoparticle is rendered hydrophobic. In one embodiment, and using a CdSe/Au particle as an example, the CdSe/Au particles are covered by trioctylphosphine oxide and alkylphosphonic acid, both of which are hydrophobic. Surface functionalization covers the Au portion of the CdSe/Au particles with polyethylene glycol, making them hydrophilic; the CdSe portion, still covered by trioctylphosphine oxide and alkyl phosphonic acid, remains hydrophobic. In one embodiment, CdSe/Au particles are suspended in N,N-dimethylformamide containing detergent (e.g., 1% Triton X-100) and exposed to polyethylene glycol-(CH2)10-SH to coordinate the thiol to the Au end. The nanoparticles may be coated with biocompatible mono- or bilayers of phospholipid a protein, a peptide polyethylene glycol (PEG) that can be used as a scaffold to aid in biocompatibility of the particle. The particles can be entirely or partially biodegradable. The particles may also be included in or coated on a bioabsorbable or non-bioabsorbable but biocompatible polymer structured or configured as a fiber, a tube, a substantially two-dimensional structure, or a three-dimensional structure to fit any anatomical or physiological site. The coated polymer structure may be any desirable length or size to maintain its position with respect to a tissue structure. The therapeutic stimulation of the polymer and adjacent tissue may stimulate and/or inhibit the excitation of cells depending upon the wavelength of the applied light and the character of the one or more types of particles associated with it, with differing parts of the polymer, e.g., the front and back sides of a substantially two-dimensional structure, having different particles in order to have different effects upon the target cells adjoining those parts. The nanoparticles may be coated with or linked to, e.g., folate, polydopamine, etc. so that these molecules are targeted intracellularly, extracellularly, to a cell membrane, to a specific cellular site or organelle, etc. The nanoparticles may be coated with a thermosensitive polymer, e.g., chitosan, PEG, etc. Application of an external energy source results in a slight increase in temperature, e.g., to about 39° C. to about 43° C. in one embodiment, to about 40° C. to about 42° C. in another embodiment, that facilitates release of the biomolecule from the thermosensitive nanoparticles or quantum dot. The nanoparticles can be made to respond to various wavelengths of light (visible and invisible). In one embodiment they are coated with organic molecules. In one embodiment, they are completely organic. In one embodiment, they are PEGylated to last longer. In one embodiment, they are coated to be attracted to certain receptors or stay only on the cell surface.

This system is programmed by a processor to define the frequency and the duration of the emitted light pulses.

All nanoparticles that are coated/targeted can be injected locally, in a body cavity or systemically, to treat various diseases.

In one embodiment one administers systemically, in a body cavity, or locally, a plurality of nanoparticles, which can include quantum dots, or plasmonic nanoparticles. The nanoparticles may be magnetic, paramagnetic, nonmagnetic, metallic, non-metallic, organic, inorganic, synthetic, piezoelectric, nanobots, lanthanide, cerium, gold, zinc, silver, silicone, perovskite; or liposomes, dendrimers, nanotubes, nanowires, caged nanoparticles, nanoshells, etc. The nanoparticles may be coated with a biocompatible polymer such as biotin, streptavidin, PEG, cell penetration peptide (CPP), arginine CPP, etc., known in the art. The nanoparticles may be conjugated with antibodies or aptamers for cell targeting.

In one embodiment, the method delivers to the cells nanoparticle-gene conjugates with opsin family genes to simultaneously stimulate the cells by light and to modify a pathology-inducing cellular DNA, either nuclear or mitochondrial DNA, with an endonuclease or a CRISPR/Cas9 conjugated with the nanoparticles. Once inside the cell, the endonuclease or CRISPR/Cas9 seeks the mitochondria or the nucleus, containing a specific DNA mutation, and destroys this disease producing DNA by excision and/or replacement with a normal DNA segment, or adding an opsin gene and/or other required genes, as known in the art.

In one embodiment, the method delivers locally or systemically nanoparticle stimulating genes, such as opsin family genes, conjugated with the CRISPR/cas9 complex to repair or replace a defective gene in the nucleus or mitochondria of retinal cells, CNS neuronal/glial cells, spinal cord cells, peripheral nerve cells, or cardiac cells.

In one embodiment, the method administers a plurality of biocompatible or biodegradable nanoparticles conjugated with opsin family genes and a CRISPR/cas9 complex systemically or locally to an intraocular site, CNS, proximate or adjacent peripheral nerves, in the heart, etc. Once the CRISPR/cas9 DNA enters the cell, it transcribes its DNA into a nuclease which scans the host DNA for a target sequence. The nuclease cuts the host DNA at the target. With one cut, the CRISPR can insert a new custom-made gene sequence; with two cuts, two CRISPRS can excise the DNA, and providing the host with a new DNA, as known in the art.

In one embodiment, the method administers nanoparticles conjugated with an opsin gene along with enolase, alpha, beta, gamma etc., or CRISPR/cas9 DNA while delivering an additional gene and growth factors to the stimulating gene, such as an opsin gene, etc. to the retina, CNS, spinal cord, peripheral nerves, or heart of a patient in need of such therapy.

In another embodiment, the method administers targeted nanoparticle conjugated genes systemically or locally by injection. Local injection may be, e.g., in the eye, vitreous cavity, brain ventricles, over or inside the brain, in the CSF, in the heart, close to peripheral nerves. This embodiment of the method provides therapy to degenerative diseases such as retinal degeneration, other retinal diseases, Alzheimer's disease, Parkinson's disease, stroke, spinal cord injuries, peripheral nerve injuries, etc. to stimulate neuronal function and growth or to treat cardiac arrhythmias or dysrhythmias.

In one embodiment, the method administers a plurality of coated nanoparticle conjugated gene and CRISPER/cas9 to modify a genetic defect of stem cells, using in vitro tissue culture to manipulate the DNA of the patient's locally harvested stem cells prior to administration to the patient. The stem cells may be, e.g., conjunctival limbal stem cells, heart stem cell, brain stem cell, spinal cord nerve stem cells, mesenchymal or ectodermal cells, etc., and enhance tissue repair.

In one embodiment, the method administers targeted piezoelectric nanoparticles either systemically or locally. The targeted piezoelectric nanoparticles can be controlled remotely by an ultrasound probe with an “off” switch to wirelessly stimulate the piezoelectric nanoparticles and produce an electrical current created by a wireless electrical stimulation of cells inside the body, changing the membrane potential/action potential of cells in contact with the piezoelectric nanoparticles.

In one embodiment, the method administers targeted coated piezoelectric nanoparticles conjugated with genes by routes including, e.g., submucosal injection, and spraying the nasal mucosa. In this embodiment, the nanoparticles travel through the olfactory nerves to various areas of the brain that can be stimulated with an externally applied ultrasound over the nose by a patient as needed e.g. in epilepsy, migraine, depression, Parkinson's disease, Alzheimer's disease, restless leg syndrome, etc.

In one embodiment, the method administers biodegradable targeted nanoparticles, conjugated with appropriate stimulatory and regenerative genes, by direct injection into an excitable organ having a brain blood barrier, thus without being degraded by serum enzymes, to be incorporated into those various excitable organs.

In one embodiment, the method systemically or locally delivers to cells an opsin gene with targeted nanoparticles using an implanted small diode having piezoelectric electric nanoparticles acting as an “on”/“off” switch of the battery supplying electricity to the diode laser. An externally located ultrasonic probe controls the piezoelectric nanoparticles, which stablishes the electric current for the diode laser, producing wireless switchable “on”/“off” laser light pulses for stimulating cells containing an opsin family gene or channel rhodopsin.

In one embodiment, the method uses a plurality of targeted nanoparticles along with biocompatible polymer-coated piezoelectric nanoparticles. Such biocompatible polymer-coated piezoelectric nanoparticles include zirconate titanate, perovskite-based, oxides, barium titanate, polyvinylidene fluoride (PVDF), etc. They are delivered systemically or locally, e.g., to the brain, spinal cord, peripheral nerves, retina, heart, etc. to stimulate the piezoelectric nanoparticles with extra-corporal ultrasound. The stimulation initiates an electric “on”/“off” response from the piezoelectric nanoparticles, which is transmitted to adjacent cells and modifies cellular membrane potential, resulting in hyperpolarization, hypopolarization, depolarization, or an action potential.

In one embodiment, the method uses a ribbon of piezoelectric nanoparticles attached to an “on”/“off” switch of a small implantable battery of a small diode. This system can initiate an “on”/“off” light laser response from the diode laser. Upon ultrasound activation, an electrical current is created in the piezoelectric nanoparticles that leads to the diode laser's battery, thus controlling activation of the diode laser light production. This initiates an “on”/“off” action potential in the membrane of excitable cells, depending on the frequency and duration of the electrical pulse.

In one embodiment of the method, piezoelectric nanoparticles are administered with nanoparticles conjugated with an opsin gene, and an implantable battery of a diode laser controllable by a programmable processor from a remote ultrasonic probe to remotely controllably stimulate laser light production and, in turn, excitable cells. The diode laser can be implanted external or internal to the skull, adjacent to an organ, adjacent to sympathetic or parasympathetic ganglions cells, adjacent to the brain, heart, eye, spinal cord, sympathetic or parasympathetic ganglion cells, peripheral nerves, etc. The system creates a remotely controlled ultrasonic “on”/“off” switch to indirectly remotely stimulate, by diode light, light-sensitive cells with the diose laser and, in turn, controlling organ function. For example, it may be used to control peripheral nerves to repair a paresis or paralysis, spastic muscles numbness, in nerve injuries, in the eye, in CNS degenerative diseases, epilepsy, depression, dementia, Alzheimer's disease, Parkinson's disease, spinal cord injury, peripheral nerve injury, etc.

The energy source to activate the particles provides ambient light, ultraviolet light, visible light, infrared light, or ultrasound radiation. In one embodiment, the particles respond to blue, red, green, or IR light. In one embodiment, a plurality of particles respond to various specific wavelengths. In one embodiment, the particles have multiple semiconductor cores, and thus respond to various wavelengths. The wavelength selections are photons with different energies. The particles must have energy bandgaps or energy statuses that match the energy of the photons. One skilled in the art tunes the energy levels using materials with different band-gaps or by carefully selecting the quantum size as it affects the energy level. Electromagnetic radiation includes infrared radiation (700 nm to 1 mm), visible light (380 nm to 760 nm), and ultraviolet radiation (4 nm to 400 nm), or stimulation may be by means of ultrasound in case of piezoelectric nanoparticles such as quartz, perovskites, etc.

All glial cells in the central nervous system (CNS) are coupled extensively by gap junctions. This coupling underlies several glial cell processes, including regulating extracellular K+ by spatial buffering, propagating intercellular Ca2+ waves, regulating intracellular ion levels, and modulating neuronal activity.

Activation of retinal glial cells with chemical, mechanical, or electrical stimuli often initiate propagated waves of calcium ions (Ca2+). These Ca2+ waves travel at a velocity of 23 m/second and up to 180 m/second from the site of initiation. The waves travel through both astrocytes and Muller cells, even when the wave is initiated by stimulating a single astrocyte.

Ca2+ waves propagate between glial cells in the retina by two mechanisms: diffusion of an intracellular messenger through gap junctions, and release of an extracellular messenger. Ca2+ wave propagation between astrocytes is mediated largely by diffusion of an intracellular messenger, likely inositol triphosphate (IP3), through gap junctions, along with release of adenosine triphosphate (ATP). Propagation from astrocytes to Muller cells, and from one Muller cell to other Muller cells, is mediated by ATP release.

Stimulated glial cells directly modulate the electrical activity of retinal and CNS neurons, leading either to enhanced or depressed neuronal spiking. Inhibitory glial modulation of neuronal spiking may be Ca2+-dependent, because the magnitude of neuronal modulation was proportional to the amplitude of the Ca2+ increase in neighboring glial cells. Besides pathologies in one or more of the above described mechanisms to maintain and/or regulate retinal cell polarity, other excitable cells besides the retina may have pathologies that occur from defects in cell plasma membrane polarization. As one example, excitable cells in the brain of Alzheimer's patients have abnormal electrical conducting and stabilizing mechanisms, resulting in loss of electrical stimulation. Repolarization of these cells provides additional stimulation to replace the abnormal cell membrane polarization and/or the cell membrane polarization that was diminished or lost. As another example, glial cell scar tissue culminating from epileptic seizures results in abnormal electrical stabilizing mechanisms in excitable cells of the brain. Repolarization of these cells provides a stabilized threshold, resulting in a calming effect. One skilled in the art will appreciate other pathologies for which the inventive method may be used. Therapeutic stimulation of the brain, spinal cord, and/or peripheral nerves may similarly be performed with implanted fiber optics, including cylindrical, tubular, substantially two- or three-dimensional branching tree-like structures, to deliver light to these tissues.

One embodiment provides nano- or micro-sized solar cells to regulate the polarity of excitable cells. As previously described, excitable cells include, but are not limited to, sensory cells such as the retina of the eye, all three types of muscle cells, and central and peripheral system nerve cells. Such nano- or micro-sized solar cells are hereinafter generally referred to as particles. In one embodiment, particles encompass any and all sizes which permit passage through intercellular and/or intracellular spaces in the organ or area of the organ of interest.

Method of Administration in the Body

In one embodiment, the particles are introduced into the central nervous system, e.g., by injection, the nanoparticles or quantum dots are covalently linked, i.e., conjugated, with natural or synthetic biomolecules (e.g., proteins, peptides, nucleic acids, oligonucleotides, etc.) that introduce retinal gene therapy bound to quantum dots/semiconductor nanowires and can be labeled for visualization, tracking, sensing, etc. For example, the quantum dots can be labeled or tagged with a signal recognition moiety. Such a vector can incorporate quantum dots using, e.g., (poly)ethylene glycol (PEG) moieties. Combinations of these embodiments are contemplated and included in the inventive method, using methods known by one skilled in the art and as subsequently described.

Delivery Vectors

In one embodiment, the vector may be a plasmid vector, a binary vector, a cloning vector, an expression vector, a shuttle vector, or a nanoparticle vector as known to one skilled in the art. In one embodiment, the non-viral vector, e.g., gold nanoparticle typically contains a promoter, a means for replicating the vector, a coding region, and an efficiency increasing region. As one non-limiting example, nanoparticles are functionalized and/or linked to (poly)ethylene glycol (PEG) moieties. The number of PEGS can be varied depending on, e.g., ocular site, need to enhanced hydrophilicity, protein size, etc. The particles may be coated with or linked to, e.g., folate, polydopamine, lipid polymer, cell penetrating peptides (CPP), activatable cell penetrating peptides (ACPP), TAT agents, etc. so that these molecules are targeted intracellularly, extracellularly, to a cell membrane, to a specific cellular site or organelle, etc. the nanoparticles are conjugated with CRISPR/cas9 and a DNA promoter delivered to be either incorporated inside the cell and activated by a source of energy, such as light, or ultrasound if the nanoparticle is piezoelectric, such as quartz, etc., to stimulate the cell membrane and deliver the appropriate gene inside the cell and escape the endosome by absorbing either the light or ultrasonic energy and deliver the gene to the cell nucleus.

Nanoparticles/QD Antibody Coating

In one embodiment, the quantum dots that are conjugated or associated with a biomolecule are delivered to a target cell cytoplasm or nucleus, using described methods and/or methods known in the art. In one embodiment, the biomolecule comprises nucleic acid, such as DNA and RNA, as well as synthetic congeners thereof. Non-limiting examples of nucleic acids may include plasmid DNA encoding protein or inhibitory RNA producing nucleotide sequences, synthetic sequences of single or double strands, missense, antisense, nonsense, on and off and rate regulatory nucleotides that control protein, peptide, and nucleic acid production. Nucleic acids include, but are not limited to, genomic DNA, cDNA, RNAi, siRNA, shRNA, mRNA, tRNA, rRNA, microRNA, hybrid sequences or synthetic or semi-synthetic sequences. Each of these may be naturally occurring or synthetic. Each of these may be any size, e.g., ranging from oligonucleotides to chromosomes. They may be obtained by any technique known to one skilled in the art.

In one embodiment, the disclosed quantum dot-nucleic acid complex additionally contains a magnetic or paramagnetic nanoparticle that facilitates introduction of the complex into a cell. In one embodiment, the complex comprises a quantum dot conjugated with a targeting moiety and a biomolecule, such as a gene, DNA, RNA, RNAi, sRNA, Np/or QD/opsin and ion channel gene and corrective gene of the cell with CRISPR/cas9, etc., and a magnetic or paramagnetic nanoparticle also conjugated with the targeting moiety. In embodiments, the targeting moiety is an antibody or a ligand for a receptor.

Location of the Light Source in the Body

In one embodiment, a fiber optic light source is implanted in a desired area of the brain, e.g., frontal lobe, parietal lobe, occipital lobe, temporal lobe, or cortex. In one embodiment, a fiber optic light source is implanted in a discrete area of the brain, e.g., basal ganglia including striatum, dorsal striatum, putamen, caudate nucleus, ventral striatum, nucleus accumbens, olfactory tubercle, globus pallidus, subthalamic nucleus; cerebellum including cerebellar vermis, cerebellar hemispheres, anterior lobe, posterior lobe, flocculonodular lobe, cerebellar nuclei, fastigial nucleus, globose nucleus, emboliform nucleus, dentate nucleus, and/or cortex including frontal lobe cortex and including primary motor cortex, supplementary motor cortex, premotor cortex, prefrontal cortex, gyri; parietal lobe cortex including primary somatosensory cortex (S1), secondary somatosensory cortex (S2), posterior parietal cortex, occipital lobe cortex including primary visual cortex (V1), V2, V3, V4, V5/MT; temporal lobe cortex including primary auditory cortex (A1), secondary auditory cortex (A2), inferior temporal cortex, posterior inferior temporal cortex; globus pallidus interna (GPi), caudal zona incerta, pallidofugal fibers, at an infarct site, at a scar tissue site, at a site in the spinal cord and/or peripheral nervous system.

In one embodiment, a controller, either internal or external controls the light source's controllable parameters. The controller operates in a manner analogous to a cardiac pacemaker that regulates cardiac rhythm. It can be adjusted or regulated by a physician as needed, either through the skin or by exposing the implanted system at an appropriate and accessible location.

Nanoparticle Size

In one embodiment, the quantum dots (QDs) or other nanoparticles have a composition and size that provides properties between that of single molecules and bulk materials, and are tunable to absorb light over the spectrum from visible to infrared energies. Their dimensions are measured in nanometers, e.g., diameter between about 1 nm to about 100 nm, preferably 1-8 nm. When combined with organic semiconductors selected to have the desired activation properties, they result in particles with selectable features. The particles can also have passive iron oxide coatings with or without polyethylene glycol coatings or positive charge coatings as commercially provided cell penetrating agents or activatable cell penetrating agents, Tat, or lipid polymers. Quantum dot solar cells take advantage of quantum mechanical effects to extract further performance.

In one embodiment, the quantum dots or other nanoparticles may be coated with organic molecules, biocompatible proteins, peptides, phospholipids, or biotargeted molecules etc., or covalently attached to polyethylene glycol polymers (i.e., they may be PEGylated) to last longer.

In one embodiment, hybrid quantum dots including but not limited to graphene/zinc oxide (ZnO) and reduced graphene oxide, or plasmonic nanoparticles coated with reduced graphene oxide, dextran-reduced graphene oxide, etc. may be used. In embodiments, ZnO is added to graphene quantum dots or to a combination of graphene particles and/or carbon nanotubes with a ZnO nanowire or nanorod using an electron gun.

In one or more embodiments, particularly those using light to stimulate the described particle, ZnO is useful because it prevents light reflecting off the particle surface, i.e., it serves as an anti-reflective coating, and provides a more efficient quantum dot compared with graphene or a carbon nanotube alone. ZnO additionally has the benefit of being an antibacterial compound and thus can be utilized for transporting biomolecules, such as DNA, along with other polymers; these may contribute a further therapeutic function and/or to the bio-compatibility of the disclosed complex.

In one embodiment, the effects of the particles on the cells can be enhanced by combining quantum dots with growth factors. Such growth factors are known to one skilled in the art, and include but are not limited to nerve growth factors, glial growth factors, nerve growth factor placenta growth factor, N-acetyl cysteine related to stem cell proliferation in vitro stimulated by light to include cell membrane polarization and depolarization to induce cell growth and proliferation.

In one embodiment, the nanoparticles are coated with silica, photovoltaic polymers and linkers or made from Zinc oxide, carbon, quartz, organic and non-organic nanoparticles, nanotubes, nanowires, nanoshells, nanocages, graphene graphene-zinc oxide, gold, iron oxide, magnetic, non-magnetic nanoparticles, perovskites, piezoelectric nanoparticles, quartz, which can be stimulated by a light source or ultrasound non-invasively, etc.

In one embodiment, the functionalized nanoparticles are graphene or reduced graphene oxide, that can be stimulated either by an electrical current or a light source non-invasively to modify the cell membrane potential.

In one embodiment, the nanoparticles are combined with Rock inhibitors, such as Fasudil (HA-1077), a selective RhoA/Rho kinase (ROCK) inhibitor, or Y-27632, small molecule inhibitor of ROCK1 and ROCK2, Simvastatin to treat the inflammatory processes in the tissue in the retina or brain in diabetic retinopathy, or Alzheimer's disease or are directly stimulated by light directly or through a fiber optic or non-invasively using an ultrasound.

In one embodiment, method of delivery to the skin or mucosa, e.g., nasal mucosa, is by spraying. Most of these applications avoid possible systemic side effects. The size of the particles allows them to easily diffuse into tissues. For neural applications other than the eye, quantum dots and/or semiconductor nanowires, either conjugated or associated with a drug, and/or administered without a drug or other agent, are administered by any route of delivery including but not limited to local, systemic, injection in the CNS, or topical by nasal routes, e.g. to control epilepsy, or Alzheimer, or stimulate the retina in retinal degeneration.

In one embodiment, nanoparticles, e.g., quantum dots, are biocompatible or rendered biocompatible, e.g., by coating with a biocompatible polymer such as (poly)ethylene glycol (PEG) moieties, e.g., quantum dot-DNA-coated polymer, or any other polymer or combinations of polymers meeting these criteria. These also include nanoparticles, e.g., quantum dots, that are biocompatible or rendered biocompatible wrapped in DNA or using peptides such as arginine-rich peptides, trans-activation transcriptional activator (Tat) peptides, biocompatible short peptides of naturally occurring amino acids that have the optical and electronic properties of semiconductor nano-crystals, e.g., short peptides of phenylalanine with nanogold, graphene, reduced graphene, graphene zinc oxide, etc., nanoparticles consisting of both inorganic or organic materials. These also include nanoparticles, e.g., quantum dots, that are biocompatible or rendered biocompatible using biocompatible mono- or bilayers of phospholipid, liposomes, etc. These also include nanoparticles, e.g., quantum dots, that are biocompatible or rendered biocompatible using a specific agent and/or coating to the nanoparticles renders them specific, e.g., a protein coating to direct nanoparticles to attach to certain cell membranes, e.g., a member of a streptavidin-biotin pair, an immunoglobulin, a member of a cell-specific antibody-antigen pair, etc. These also include nanoparticles, e.g., quantum dots, to enter a cell to increase the membrane potential of the cells to which they come into contact, that are biodegradable either entirely or partially, that are non-biodegradable, and/or that are a combination of organic and metallic quantum dots. These include nanoparticles, nanotubes, nanowires, nanocrystals such as cadmium/selenium (Cd/Se), and particular types of each, e.g., graphene quantum dots, graphene-oxide quantum dots, graphene-zinc oxide quantum dots, graphene nanotubes, reduced graphene oxide and/or carbon nanotubes.

As one example, PEG grafted polyethylenimines (PEI) encapsulate and solubilize luminescent quantum dots through direct ligand-exchange reactions via positive charges and a proton sponge effect. PEG improves nanoparticle stability and biocompatibility, as previously disclosed, and reduces PEI toxicity to cells, as well as facilitating cell penetration.

One embodiment is a composition of a nanoparticle, such as a quantum dot, that is conjugated to an activatable cell penetrating peptide (ACPP). The quantum dot or nanoparticle may be cleavably conjugated to the ACPP by, e.g., a linker (e.g., ethylene glycol moiety, PEG moiety). In one embodiment, the quantum dot or nanoparticle is labeled with a label such as a fluorescent moiety, chemiluminescent moiety, etc. In one embodiment, the ACPP is labeled with a polycationic cell-penetrating peptide (CPP). ACPP and CPP TAT and lipid coating may be naturally-occuring or artificially constructed protein segments (<30 amino acids) rich in arginine, lysine, cysteine, histidine, ornithine, etc.; preferably α-helices and about 17-amino acids. The ACPP and CPP may include a penetration accelerating peptide sequence (Pas) or an INF7 fusion peptide sequence. CPP and/or ACCP can be linked to cargoes either covalently or noncovalently, or can use block copolymers to form various kinds of micelles.

Gene(s) Transfer

One example of this enhanced penetration uses genes capable of modifying cell polarization and, potentially, creating an action potential upon specific prompts. Such genes encode, e.g., a cell membrane ion channel protein or transporter, e.g., a G protein-coupled receptors. Specific examples include genes encoding opsin family members such as rhodopsin, photopsins, halorhodopsin, and a membrane ion channel gene; the corrected muted gene or elimination of the mutated gene(s) are done using CRISPR/cas9 or genes encoding neurotransmitters such as glutamate/aspartate transporters, GABA transporters, glycine transporters, monoamine transporters such as dopamine transporter, norepinephrine transporter, serotonin transporter, and vesicular monoamine transporters. Upon exposure to light of a specific wavelength, quantum dots coated or otherwise associate with organic or non-organic biodegradable compounds may be used.

Such agents include silicon, porous silicon, aliphatic biodegradable polymers, etc. The quantum dots range from about 1 nm to 200 nm in one embodiment, and range from about 1 nm to 10 nm in another embodiment. The genes, once in the nucleus, undergo transcription and translation into the specific proteins or protein channels.

Other Agents Linked with Nanoparticle/Gene Complex

Agents may be linked to, associated with, complexed or conjugated with nanoparticles using linking agents and methods known in the art. These include, but are not limited to, the following: amino groups, carboxyl groups, S—S deprotected sulfhydril groups in biomolecules.

In one embodiment, the inventive method is used in a patient with a neurological disorder. While described in detail for use in a patient with epilepsy, which is a common neurological disorder, epilepsy stroke of traumatic brain or spinal cord injury requiring treatment, the inventive method is not so limited and encompasses any neurological disorder of the central and/or peripheral nervous system (e.g., Alzheimer's disease). Epilepsy is thus used an exemplary but non-limiting embodiment of use of the method.

Application of the Technology in Epilepsy and Other Brain Related Issues

Nanoparticles/opsin genes are useful in providing repeated electric pulses either to the brain, spinal cord, or isolated nerve cells that are involved with various neural disorders. In disorders involving these regions, low level brain, spinal cord, etc. neural pulses are not passing through for one reason or another, e.g., synapses, scar, misdirection, etc., and are released either as a giant pulse or can circuit back and forth until the membrane potential is completely exhausted. Therefore, a pulsed stimulation by an external source, such as light or electric pulses applied to the brain, ventricles, spinal cord, cerebrospinal fluid, having quantum dots and/or semiconducting nanowires or membrane ion channel gene and the corrected muted gene or the elimination of the mutated gene(s) using CRISPR/cas9 would eliminate an avalanche of the pulses in posttraumatic epilepsy, restless leg syndrome, spinal cord epilepsy, etc. A version of this concept could be potentially used to modify brain waves needed for sound sleep, alleviation of depression, etc. Stimulation of the olfactory nerve after administration of opsin gene and membrane ion channel gene and the corrected muted gene or elimination of the mutated gene(s) using CRISPR/cas9 can enhance neuronal regeneration in the brain alone or along with administration of modified stem cells with opsin/membrane ion channel gene and the corrected muted gene or elimination of the mutated gene(s), such as Presenilin1-2 using CRISPR/cas9 in aging adults, e.g., in Alzheimer's disease, Parkinson's disease or slow their progression and enhance tissue repair.

Instrumentation Light Source Location

In one embodiment, an eye tracker is used in combination with a light source to therapeutically stimulate particles provided to the eye. A small digital camera may be mounted on the patient's head, e.g., in eyeglasses, to obtain video images of the eye and transmit the images to a computer. The video images may include reflected infrared, visible, and/or ultraviolet light reflected from the eyes and captured by the camera. The video images may be analyzed to determine the average frequency of saccadic movement of the eye for an interval of time, and to compare the average frequency to one or more criteria for apparently normal or abnormal brain function. The light source, e.g., LED or low powered laser, may be activated to stimulate the particles administered to the brain or inhibit an action potential response in the brain at a predetermined frequency using physician-determined pulses of light for predetermined durations at predetermined repetition intervals. The light source in one embodiment emits light that is reflected into the eye through a stationary or rotating mirror positioned within the visual field of the eye. This system is designed to respond to detected signs that a seizure is about to begin, permitting customized response patterns that may provide a degree of seizure control or the brain stimulation can be done through the nasal mucosa or through the skin at the base of the nose externally with both light and ultrasound.

In one embodiment, equipment similar to that previously described may be used to provide enhanced vision to a patient, e.g., a patient having damaged or diseased outer photoreceptor segments. A small digital camera may be mounted on the patient's head, e.g., in eyeglasses, to obtain video images. In this embodiment, however, the video images are obtained from the viewpoint and across the visual field of the patient, i.e., are images of the external environment, rather than of the eye itself. The images may approximate those viewable using only visible light or be hyperspectral images including infrared, visible, and/or ultraviolet wavelengths. The light source, emitting at least one wavelength of light, may be activated to stimulate the particles administered to the eye in a pattern representative of the video image. For example, color images are typically represented as a combination of images in three primary colors, but may be converted to a combination of images in only two colors or a single image varying only in relative intensity. Particles adapted to specifically bind to one or more of the S-cone, M-cone, and L-cone photoreceptor cells may be activated by pulses of different wavelengths to stimulate the perception of colors. Particles adapted to bind to photoreceptor cells generally, rods, or alternate targets in signaling pathway, such as photoreceptor cell body, bipolar ganglion cells, amacrine cells, and Muller cells, may be activated by pulses to stimulate the perception of intensity, i.e., to simulate vision under low-light conditions. In one embodiment, placement of the photovoltaic particles in the membrane mimics the naturally-occurring amphiphilic transmembrane proteins, which have hydrophobic membrane-spanning domain(s) that interact with fatty acyl groups of the membrane phospholipids and hydrophilic domains extending into the aqueous medium on each side of the membrane. An embedded nanoparticle, e.g., with the metal portion inside the cell, acts as a photovoltaic cell where the electric current varies with the rate of photon absorption. Illumination of embedded particles generates a photovoltaic that reduces the potential across the cell membrane by about 10 mV. Such membrane depolarization causes enough voltage-sensitive Na+ ion channels to open to generate an action potential that travels down the axon.

Instrument and Light Source

The stimulated photoreceptors will transmit the stimulated pulses to the optic nerve and to the brain, where the pulses will be interpreted as images by the visual cortex. The light source may be a complex source, e.g. a small scale LCD or OLED screen positioned in front of the eye, e.g. as a lens of glasses, or to reflect from a stationary mirror positioned within the visual field of the eye. The light source may alternately be single or multiple wavelength scanned-beam system, using one or more discrete light sources, e.g., LEDs or low power lasers, and a rotating mirror to stimulate, pixel by pixel, the photoreceptor cells, the outer segment of the retina, the inner segment of the retina, etc., similar to the manner in which an electron gun excites the phosphors of a cathode ray tube television. The computer may manipulate the image size, intensity, contrast, etc. to improve visibility, as well as to translate between detected wavelengths of light, e.g., the typical red, green, and blue color-filtered detectors employed in Bayer filtered sensors or multi-sensor imaging blocks, and emitted frequencies of light emitted at the appropriate wavelengths to stimulate the one or more types of particles. The particles in the retina can respond to both detection of IR light that is reflected from a real object that acts on the particles, or detection of IR light that is captured by a digital camera and is reemitted by a head-mounted device, with the camera and processor able to amplify the pulse frequency, energy, etc.

Eye Application and Diseases

Upon administration, the particles are disseminated and/or located intracellularly (within a cell), intercellularly (between cells), or both intracellularly and intercellularly. They may be administered in a number of ways. With respect to the eye, they may be injected through the retina, under the retina superiorly, over the fovea, through the outer plexiform layer down to the fovea, into the vitreous cavity to diffuse through the retina, in the lens capsule after cataract surgery to diffuse out gradually in the post-operative period etc. The procedure permits particles to be located at any site including the macula, that is, the particles may be directly on the macula, directly on the fovea, etc. distinguishing from procedures requiring electrodes to be located beyond the macula or beyond the fovea so as not to block foveal perfusion. The procedure does not require major invasive surgery and is only minimally invasive, in contrast to procedures that involve surgical implantation of an electrode or photovoltaic apparatus. The procedure locates particles diffusively substantially throughout the eye, or selected regions of the eye, in contrast to procedures in which an electrode or other device is located at a single site. Thus, the site of treatment is expanded with the inventive method. In this way, the particles locate within excitable cells, such as the retina, macula, etc. using an ocular example, and also locate between these excitable cells, and are thus dispersed substantially throughout a region of interest. Particles not located as described are handled by the retinal pigment epithelium.

In one embodiment, the retinal or other cell so modified by the method contains a light-sensitive protein that itself may be excited directly by light of a specific wavelength, or in an alternative embodiment, be excited by light of a different wavelength or produced by the quantum dot (e.g., fluorescence) after the quantum dot is excited upon exposure of light. For example, if the modified genes of the cell produce halorhodopsin, then the quantum dots to which the halorhodopsin-encoding gene were associated can be excited to then activate the halorhodopsin to silence the cell. If the modified genes of the cell produce channelrhodopsin, then the quantum dots to which the channelrhodopsin-encoding genes were associated can enhance an action potential. As known to one skilled in the art, channelrhodopsins, a family of proteins, function as light-gated ion channels in controlling electrical excitability among other functions. As known to one skilled in the art, halorhodopsin is a light-activated chloride-specific ion pump. When quantum dots are combined with channelrhodopsins or halorrhodopsons, quantum dots enhance the effects of these proteins, and result in enhanced cell polarization responsive to light stimulation.

Other Nanoparticles

It will be appreciated that nanoparticles are not limited to the previously described quantum dots and nanotubes, and include other carbon-based skeletal-type structures. Examples of such structures encompassed by nanoparticles and/or nanostructures include, but are not limited to, fullerenes, bucky balls, micelle-like structures such as micellar nanoparticles (MNP), lipid-based liposomes, dendrimers, and single-stranded deoxyribonucleic acid- or ribonucleic acid-oligonucleotide aptamer-conjugated or modified nanoparticles conjugated with opsin and membrane ion channel gene and the corrected muted gene(s) using CRISPR/cas9.

Gene Delivery Vector and CRISPR/cas9

In one embodiment of the inventive method, such nanoparticles function as carriers and deliver a variety of opsin gene families. Opsin family genes include rhodopsin, halorhodopsin, photopsin, and channelrhodopsin. As known in the art, such gene families can integrate into the cell nucleus and nucleolus. The opsin gene families can be delivered alone, or in combination with one or more other genes to correct a genetic defect of excitable cells, and membrane ion channel gene and the corrected muted gene or elimination of the mutated gene(s) using CRISPR/cas9 or to induce an action potential in a membrane of excitable cells.

In one embodiment, the method is performed on a patient having a condition involving a defective gene, and where the condition is in a relatively early stage, amenable to therapy by the inventive method. The method results in stimulation of cells and prolongation of a regenerative process. The method may permanently repair the condition when the defective gene is included with the nanoparticle containing the opsin-family gene.

The nanoparticle-gene(s) composition may be administered intraocularly by intravitreal, intraretinal, subretinal, or other site of injection such in the CSF or at the site of spinal cord injury, peripheral nerve injury or cut, in the brain after stroke in Alzheimer's disease, Parkinson's disease etc., topical application, nasal mucosa, cornea and conjunctiva; however the nanoparticles are stimulated with external light or ultrasound non-invasively. The nanoparticle-gene(s) composition with opsin and membrane ion channel gene and the corrected muted gene or elimination of the mutated gene(s) using CRISPR cas9 may be administered intrathecally into the cerebrospinal fluid, brain, or spinal cord. Intrathecal injection protects the gene(s) such as presenilin1 or presenilin 2, that otherwise may be damaged by contact with fluid and/or cellular blood components. The nanoparticle-gene(s) composition may be injected in any location, e.g., heart, peripheral nerves, etc., with a small gauge insulated metallic needle connected to a battery to carry electricity in the tissue for electroporation delivery inside the desired cells if needed. Other types of force may also be simultaneously applied to enhance cell penetration of the nanoparticle. These embodiments facilitate penetration of the nanoparticle-gene(s) complex into cells. Subsequent stimulation of the cells is achieved using a fiber optic or ultrasound pulse to stimulate the brain, retinal cells, olfactory or optic nerve, peripheral nerves, cardiac muscle, skeletal muscle, etc. non-invasively, and electrical stimulation may be replaced with light stimulation using a diode laser or ultrasound.

The nanoparticles may further be encapsulated to protect their contents. As only one non-limiting example, the dendrimer poly(amidoamine) (PAMAM) can be functionalized to be biocompatible and cell penetrating. Other dendrimers are poly(amidoamine-organosilicon) (PAMAMOS), poly(propyleneimine) (PPIO), tecto, multilingual, chiral, hybrid, amphiphilic, micellar, multiple antipen peptide, and Frechet-type dendrimers. Dendrimers have been proposed as carriers of luciferase gene, but there are no reports using or suggesting their use to transfer opsin gene family members and membrane ion channel gene and the corrected muted gene or elimination of the mutated gene(s) using CRISPR/cas9, activated by light and producing an action potential on cells. The nanoparticles may be rendered visible, e.g., by combining them with other nanostructures such as functionalized quantum dots.

Fullerenes and buckyballs are nano-sized three dimensional carbon molecules having hollow spherical, oval, or tubular structures. Dendrimers are highly symmetrical nano-sized spherical compounds composed of branched polymers that have many functional groups on their outer surface. They are made with variable functionality, thermal stability or solubility, and are commercially available or synthesized as known in the art.

The nanoparticles have a small size of up to about 1 nm. The combined nanoparticle-gene(s) complex may likewise have a small size or up to 3 nm to 800 nm or more, as long as they are able to pass through intracellular spaces of the retina or CNS.

In one embodiment, administration of the inventive method into stem cells causes cellular multiplication upon light stimulation, with the stem cells becoming activated after light pulse exposure. Stem cells treated with a light controllable gene(s), such as opsin gene and CRISPR/cas9 administration and other membrane channels genes and CRISPR/cas9 with simultaneous repair of the defective gene and CRISPR/cas9 makes them more responsive to low level of light that would not be the case with application of opsin gene alone which build only channel rhodopsin in the cell membrane. After stimulation of the stem cells polarization and depolarization of the cell membrane produced by light pulses or ultrasound causes these cell to proliferate and grow in vitro or after their administration in vivo in process of their migration to replace cellular loss in a tissue such as brain or retina, spinal cord, etc. and enhance tissue repair. This embodiment could be used for therapy in pathologies such as, e.g., age related macular degeneration, genetic disease of the retina, or brain or after spinal cord injury or stroke, Alzheimer's disease or Parkinson's disease and other neurodegenerative diseases of the brain and organs, such as heart, injured nerves, etc. Light is delivered to the desired site using a fiber optic whereas these cells or organs can be also stimulated with ultrasonic pulses applied closed to the internal site non-invasively through the skull, skin or mucosal area, such as olfactory nerves, eye after administration of the stem cells or the nanoparticles/genes and enhance tissue repair.

In one embodiment, a method to enhance functional recovery of a cell in a patient in need thereof includes administering a combination of opsin/CRISPR/cas9 with ion channels genes and CRISPR/cas9 using graphene quantum dots, graphene-oxide quantum dots, graphene-zinc oxide quantum dots, graphene nanotubes, and/or carbon nanotubes, dendrimers, dendrimers conjugated with CPP or ACPP collectively termed nanoparticles, as non-viral gene carrier or non-viral vectors combined with cell penetrating agents, CPP or ACPP, to a site in a patient where functional cell recovery is needed.

In one embodiment, the nanoparticles at the site along with rhodopsin/halorhodopsin and an ion channel gene and CRISPR/cas9 are controllably activated by light, thus controllably altering a cellular electrical property along with ion channel which independently enhances the cell polarization. Activation uses either an internal or external device or a natural light source, a fiber optic comprising wires and a tip containing a light source, a sensor connectable to the fiber optic wires, and a controller to receive and generate electrical signals. Signals resulting from the altered cellular electrical property at the site are sensed and are optionally provided to a processor to monitor and/or controllably alter the electrical property using the controller. The processor may be implanted in the patient, e.g. under the skin, or may be external to the patient suffering from a degenerative process of the retinal or brain or after a vascular incidence.

In one embodiment, the method may be used with neurons, muscle cells, cardiac cells, ocular cells, etc. on any cell that would benefit from such controlled therapy. As an example, one candidate is a patient with a neural-related pathology, a neurodegenerative disease or symptom of such a disease, or blindness, and/or surgically injured neurons such as traumatic brain injuries (TBI) or a chronic inflammatory process as is the case in Alzheimer's disease. The hallmark of both conditions are an inflammatory response produced as a result of low impact trauma or an infection such as low grade infection such as herpes, or cytomegalovirus inducing leakage of plasma through the brain or retinal capillaries or in diabetes disease manifested as chronic vasculopathy in the retina or brain leading to diabetic retinopathy loss of endothelial cells leakage in the retina or the brain enhancing dementia in these patients.

In one embodiment, the patient is administered antibody coated nanoparticles of 1-10 nm that pass easily through the damaged retinal and brain capillaries and are coated with a polymeric coating carrying anti-inflammatory agents, such as Rock inhibitors such as fasudil, botulinum toxin at low doses or picogram concentrations that have both an anti-inflammatory effect and anti-TGF beta effect located at the site of Alzheimer plaques, thus reducing the production of Tau entanglement and neurofibrillary elements seen in Alzheimer's disease plaques or TBI.

In one embodiment, the patients benefiting from such therapy include those with epilepsy, Parkinson's disease, Alzheimer's disease, depression, spinal cord injury, peripheral nerve injury, stroke, and chronic pain. The pluralities of antibody coated nanoparticles complex with Rock inhibitors, opsins and membrane ion channel genes and CRISPR/cas9 may be targeted or provided at a site of brain injuries to controllably enhance neuronal growth.

In one embodiment the nanoparticles contain other agents, such as Rock inhibitors to reduce the inflammatory components of these diseases and other agents to facilitate neuronal growth, e.g., potent ROCK inhibitor; orally bioavailable Fasudil hydrochloride, inhibitor of cyclic nucleotide dependent- and Rho-kinases GSK 269962, Potent and selective ROCK inhibitor GSK 429286, Selective Rho-kinase (ROCK) inhibitor H1152 dihydrochloride, selective Rho-kinase (ROCK) inhibitor Glycyl H 1152 dihydrochloride, selective Rho-kinase (ROCK) inhibitor; more selective analogue of H1152, cell-permeable, selective Rho-kinase inhibitor OXA 06 dihydrochloride, potent ROCK inhibitor PKI1447 dihydrochloride, potent and selective ROCK inhibitor; antitumor SB 772077B, potent Rho-kinase inhibitor; vasodilator SR 3677 dihydrochloride, potent, selective Rho-kinase (ROCK) inhibitorTC-57001, potent and highly selective ROCK inhibitor; orally active Y-27632 dihydrochloride.

In one embodiment, the nanoparticles contain other agents, such as Rock inhibitors to reduce the inflammatory components of these diseases and other agents to facilitate neuronal growth, e.g., potent ROCK inhibitor, myelin basic protein (MBP), valproic acid, ketamine, donepezil hydrochloride, thymosin β10, thymosin α1, choline acetyl esterase, nerve growth factor (NGF), and/or brain derived growth factor (BDGF). As another example, one candidate is a patient with cardiac dysrhythmia, with the nanoparticles provided and controllably in need of a pacemaker, etc. Other agents may be included, e.g., stem cells, immunomodulators, anti-vascular endothelial growth factor (VEGF) agents, anti-integrin agents, anti-inflammatory agents, such as fasudil, antibiotics, etc.

In one embodiment, where a genetic disease exists, the genetic defect of the stem cells are corrected in vitro using CRISPR/cas9 prior to the administration of the stem cells. One embodiment is a method for delivering antibody coated nanoparticles such as gold, graphene or piezoelectric pegulated nanoparticles such as quartz, a Rock inhibitor to inhibit pro-inflammatory components of the disease and counteracting reactive oxygen species which affect the cellular telomeres and an opsin family gene and CRISPR/cas9 or in combination with presenilin1 or presenilin 2 gene family and an ion channel membrane gene, to an anatomical and/or physiological site for stimulating, modifying polarization of, and/or inducing an action potential in a cell at the site and enhance tissue repair.

In another embodiment, one administers a complex comprising a non-quantum dot nanoparticle carrier, such as a dendrimer, etc., a biocompatible molecule, CPP or ACPP for cell uptake of the complex, an opsin family gene, an ion channel family gene and CRISPR/cas9 in a biocompatible fluid to the anatomical and/or physiological site, to result in formation of a light activated channel in a cell membrane permitting cell stimulation by an external or internal light transmitted by a fiber optic to modify polarization of, and/or induce an action potential in the cell at the site. Optional components include a targeting moiety such as an antibody, antigen, ligand, receptor, etc., and/or a third gene to replace the defective gene in the cell along with Clustered Regularly interspaced Short Palindromic Repeats (CRISPR) Cas 9 or controlling the CRISPR with an amino acid switch Lysine (BOC) by expanding the genetic code.

In one embodiment, opsin family gene members include rhodopsin, halorhodopsin, photopsin, and channelrhodopsin and a membrane ion channel gene, and the corrected muted gene or elimination of the mutated gene(s) using CRISPR/cas9. The second gene is used to encode a membrane ion channel and a third gene where the patient has a genetic and CRISPR/cas9 or acquired degenerative disease or condition, where the second gene ameliorates at least to some extent the disease or condition by providing the component that is lacking, by enhancing gene activity, by silencing gene activity, etc., as known in the art. Examples include a degenerative retinal condition, a degenerative central nervous condition, etc.

In one embodiment, a systemic disease and/or a degenerative cardiovascular disease are targeted. The third gene may encode a protein, or it may be an inhibitory RNA (RNAi) for gene silencing.

In one embodiment, the method may be used on an excitable cell, such as a retinal cell, astrocytes glial cells, a cardiac cell, a muscle cell, a central nervous system cell (CNS), a spinal cord cell, and/or a peripheral nerve cell. The method may also be used on a non-excitable cell, such as a fibroblast, a glial cell, a stem cell, and/or a pluripotential mesenchymal stem cell. The complex may include an opsin gene, a membrane ion channel gene, and/or the corrected muted gene using CRISPR/cas9. In one embodiment, light-induced cell stimulation may induce cell proliferation in the stem cells or cells that are growing, which desirably replaces cell loss due to various diseases or conditions at the targeted site. Examples of such conditions include, but are not limited to, age related macular degeneration, stroke, and ischemia spinal cord injury or traumatic brain injuries.

In one embodiment, the functionalized quantum dot/nanoparticles are administrated to an excitable cell, such as a retinal cell, astrocytes glial cells, a cardiac cell, a muscle cell, a central nervous system cell (CNS), a spinal cord cell, and/or a peripheral nerve cell in vitro or in vivo. The method may also be used on a non-excitable cell, such as a fibroblast, a glial cell, a stem cell, and/or a pluripotential mesenchymal stem cell. The complex may correct a mutated gene using CRISPR/cas9 or PNA. In one embodiment, in vitro or in vivo, in these QD/cells complex, the pulsed light-induces cell stimulation and proliferation in the stem cells via the quantum dots inducing cell membrane polarization/depolarization, when the light is on or off intermittently at a rate of <30 pulse/sec. which desirably replaces cell loss due to various diseases or conditions at the targeted site. Examples of such conditions include, but are not limited to, age related macular degeneration, stroke, and ischemia spinal cord injury or traumatic brain injuries, or Alzheimer's disease or immune cells.

In one embodiment, the complex may be administered by an injection at an intraocular, intravitreal, intraretinal, subretinal, or intrathecal location. Its entry or penetration into the target cell may be enhanced by electroporation or mechanical force. In one example, the complex is injected in a desired location with an insulated metallic needle connected to a power source for electroporation delivery of the complex inside the cell.

In one embodiment, combination mechanisms to correct, reduce, and/or prevent physiological electro-sensory damage or electromotor damage and promote functional recovery of excitable cells, e.g., neurons in the central nervous system (i.e., brain and spinal cord) and neuronal cells involved with visual, auditory, vocal, olfactory responses, e.g., retinal cells in the eye, cochlear cells in the ear, olfactory cells in the nose, etc., and neurons in the peripheral nervous system are provided. The inventive combination methods can be thought of as akin to combination approaches in treating neoplastic lesions, but targeting less than optimally-functioning excitable cells. The combination mechanism may also be used to correct, reduce, and/or prevent damage to tissues by rendering normally non-excitable cells in proximity to partially or wholly non-functional cells artificially functional.

In one embodiment, the combined method promotes functional recovery and controllably regulates plasma membrane polarization of a functional excitable neuronal cell. A biomolecule effecting gene therapy is administered into an eye and/or central nervous system of a patient in need of the therapy (e.g., a patient with a neuronal degeneration).

In one embodiment, the rhodopsin/halorhodopsin and membrane ion channel genes can be administered in the solution with groups of nanoparticles or individually along with plurality of nanoparticles in a physiological solution along with CRISPR/cas9 for adding the missing gene(s) or removing a defective gene etc.

In one embodiment, quantum dots and/or semiconductor nanowires, nanoparticles, dendrimers (generically referred to hereafter as particles or solar cells) are conjugated with cell penetrating agents and Rock inhibitors and appropriate genes, CRISPR/cas9 and DNA promoters using homology directed repair (HDR), and are administered into the eye having a genetic disease, by topical application to the conjunctiva or injection in the vitreous or under the retina, etc. and/or central nervous system by administration topically to the nasal mucosa, or in cerebrospinal fluid of the patient, either simultaneously or sequentially to stimulate the excitable cells after the biomolecule is administered using light or ultrasound applied externally through the skin at the base of the nose.

In one embodiment, quantum dots are nanoparticulate semiconductors, and they are administered with opsin genes, ion channel gene(s) using CRISPR/cas9 mediated Homology-Independent Targeted Integration (HITI) or Homology Directed Repair (HDR) in which excitation is confined in all three spatial dimensions. Semiconductor nanowires are nanoparticulate semiconductors, in which excitation is confined in two of the three spatial dimensions, with a nanoscale diameter but a length to width ratio of 100:1 or more. Semiconductor nanowires tend to be more efficient than quantum dots in converting electromagnetic radiation into electrical charge and more similar to solar cells in creating electromagnetic fields when stimulated by such radiation.

In one embodiment, the hetero-junction creates a Schottky junction, where illumination creates electron-hole pairs that separate under the influence of the built-in field, thereby yielding a photovoltaic effect across the structure. Light is applied to the eye or central nervous system to controllably activate the particles by controlling exposure time, exposure intensity, exposure site, etc. to controllably regulate the plasma membrane polarization of the functional excitable cell.

In one embodiment the nanoparticles are coated with known biocompatible polymers such as PEG, streptavidin, biotin, cell penetrating peptides, antibody to the specific cell membrane receptor, etc. These polymers are then conjugated with the opsin gene and/or ion channel gene.

In one embodiment, the nanoparticles/gene complex is conjugated with CRISPR cas9 and a DNA promoter for adding a gene or removing a gene etc. using CRISPR/cas9 mediated Homology-Independent Targeted Integration (HITI) or Homology Directed Repair (HDR).

In one embodiment, the light sensitive particles/ion channel gene alone or in combination with opsin genes or ion channel genes and CRISPR/cas9 may be provided to specific neurons or tissue such as retina, CNS, spinal cord peripheral nerves, olfactory nerve or heart for therapy. Opsin genes, in absence of quantum dots work as light absorber chromophores that initiate the depolarization which is then enhanced by an ion channel gene to be transported to the next excitable cell. While quantum dots may be eliminated from the cells over time, the opsin genes and Ion channel genes remain in the neuronal cells which do not divide permanently as long as the cell is alive. However, if needed over time, repeated administration is possible since there are no viral proteins or viral genes in the tissue as is the case with the use of viral vectors. Repeated injection or administration with the described invention is possible without causing immune response to a foreign viral protein.

In one embodiment, the biocompatible solution containing cell penetrating peptides (CPP) or activatable cell penetrating agents (ACPP), nanoparticles/opsin gene/ion channel complex CRISPR/cas9 is injected in the vitreous cavity to pass through the retinal intercellular spaces and picked up by the retinal ganglion cells, astrocytes, glial cells and Muller cells, retinal pigment epithelium.

In one embodiment, the biocompatible solution containing cell penetrating peptides (CPP) or activatable cell penetrating agents (ACPP), nanoparticles/opsin gene/ion channel complex and CRISPR/cas9 is applied topically as drop or spray, etc. to the cornea conjunctiva, mucosa, skin, nasal mucosa, to be picked up and transferred to the appropriate cells that have the receptor to the antibody coated nanoparticles.

In one embodiment the pluralities of nanoparticles, such as dendrimers/ion channel genes alone or in combination with dendrimers/opsin genes and/or ion channel genes and CRISPR/cas9 may be provided to specific neurons or tissue for therapy such as specific neurons or tissue such as retina, CNS, spinal cord peripheral nerves, olfactory nerve, or heart for therapy.

Opsin genes in presence of quantum dots/piezoelectric nanoparticles, or gold nanoparticles work as light absorber chromophores that initiate the membrane depolarization or an action potential which is then enhanced by ion channel genes to be transported to the next excitable cell or regulate the membrane potential of the cells after light or ultrasound exposure.

In one embodiment, the pluralities of nanoparticles, such as dendrimers/ion channel genes alone or in combination with dendrimers/opsin genes and/or ion channel genes with CRISPR cas9 may be provided to an optic nerve for retinal therapy.

In one embodiment, the pluralities of nanoparticles such as dendrimers/ion channel genes alone or in combination with piezoelectric nanoparticles, dendrimer/opsin genes or ion channel genes along with CRISPR/cas9 may be provided to an olfactory nerve through the nasal mucosa nasal nerve therapy, and/or as a point of entry for brain therapy, etc. As another example, they may be provided to selective or non-selective sites for selective stimulation of various regions, either alone or in combination.

In one embodiment, as non-limiting examples of selective stimulation of central nervous system nerves, the visual cortex can be stimulated through specific light stimulation of the retina, the olfactory neuron can be stimulated by smell, the auditory neuron can be stimulation by sound, etc. As non-limiting examples of selective stimulation of peripheral nervous system nerves, chronic pain may be controlled by direct stimulation of the appropriate nerves, and appetite may be suppressed by direct stimulation of appropriate nerves.

In one embodiment, stimulation by light may be achieved by several mechanisms, as known to one skilled in the art. For example, using activation of particles in the brain as an exemplary, non-limiting example, activation may be provided by a fiber optic device surgically placed at the desired area of the brain, located under the scalp, and illuminated by a light source, e.g., a light emitting diode (LED) through a small window made in the skull replaced by clear glass at a desired area. Such a window may remain hidden under the skin, because it is known that light can penetrate a few millimeters into skin. An analogous concept may be used for stimulating other areas of the central nervous system, the peripheral nervous system, or heart or other muscles, with or without application of a fiber optic device if nanoparticles/gene/CRISPR/cas9 and medication are injected through an opening into the superficial area of the brain, nerve, heart muscle, etc. Such stimulation may controllably regulate, i.e., activate/deactivate, by using an appropriate wavelength of light, with or without a processor with the specific neuronal code as pulses. Quantum dots and/or semiconductor nanowires/opsin gene and ion channels may be used in conjunction with stem cell therapy or in conjunction with other devices, e.g., prosthetic devices, that are activated or otherwise rely on and/or electrical current.

In another embodiment, in addition to using the method with opsin and membrane ion channel gene(s) for the above indications and for treatment of retinal degeneration, etc., the method may also be used for posttraumatic epilepsy, amelioration of the underlying pathology and/or symptoms of genetic and/or degenerative diseases, e.g., retinitis pigmentosa, retinal degeneration, central nervous system pathologies such as Alzheimer's disease and Parkinson's disease, dopamine-regulated disorders such as migraines, autism, mood disorders, schizophrenia, senile dementia, sleep disorders, restless leg syndrome, depression, Tourette consequences of infectious diseases, epilepsy, paralysis, and traumatic injury of the brain and/or peripheral nerves.

In another embodiment, nanoparticles or semiconductor nanowires/ion channel genes and opsin alone or along with other genes may be targeted to dopamine-regulated nerves for therapy of migraines, mood disorders, for deep sub-thalamic, cerebral, or cortical and peripheral nerve stimulation for therapy of Parkinson's disease, etc.

In another embodiment, non-viral vector (e.g., dendrimer, QDs, etc.) can provide the biomolecule, which can be a natural or synthetic protein, peptide, nucleic acid, oligonucleotide, gene and CRISPR/cas9, etc. when conjugated with the particles. In one embodiment, the biomolecule is a cell membrane ion channel protein, such as rhodopsin, halorhodopsin, or other light-activated membrane ion channel protein.

In one embodiment, the biomolecule is a cell membrane ion channel gene that cannot be activated by itself by light, but in combination with opsin gene by increasing the extracellular potassium ion shows strong action potential with natural light stimulation in the nature. The opsin gene works as initiator of cell depolarization and other membrane ion channels complement the membrane rhodopsin of halorhodopsin. Whereas administration of opsin, e.g., to the brain ganglion cells alone, cannot be stimulated by natural light and requires a light with sufficient\high energy that cannot be tolerated, e.g., by retina or brain ganglion cells because it produces a thermal effect of overheating and coagulation in the cells and exceeding the permissible light energy level by the FDA<4000 lux. Therefore, the combination of opsin, ion channel gene, and CRISPR/cas9 complement each other's effect in the cell membrane that are by naturally excitable cells such as photoreceptor ganglion cells of the retina, brain, and muscle cells of the heart or even non-excitable cells such as fibrocytes. Thus, the combination of opsin gene, ion channel gene, and CRISPR/cas9 when applied with a non-viral vector to a cell, such as fibroblast or mesenchymal cells which are not normally excitable by light, makes the cells become excitable by the described methodology and their action potential can be regulated and controlled by a processor connected to the light generating unit, e.g., a diode laser. The embodiment achieves a synergistic effect of both genes.

In one embodiment, the combination of gene therapy can also achieve a silencing of the action potential in the target cells, e.g. cells having the rhodopsin gene, and silencing other cells carrying halorhodopsin which can be stimulated be another wavelength of light. In one embodiment, the biomolecule, e.g., membrane channel protein, is excited by the same wavelengths of light that also excite the QD particles. In one embodiment, the biomolecule, e.g., membrane channel protein, is excited by a different wavelength of light than that exciting the QD particles. These variations can increase or reduce or suppress the action potential in some cells and increase in the others, and vice versa. In all cases, the “tunable” selection of the biomolecule and the particles, as well as the specific excitation energy (typically light but also ultrasound radiation energy if piezoelectric nanoparticles are used) applied, provides a controlled and regulated process. In turn, the selective on or off activation of the genes complex provides the high degree of control that enhances efficacy and safety and permits close monitoring and regulation.

In one embodiment, nanoparticles or semiconductor nanowires/ion channel genes and opsin and ion channel gene/CRISPR/cas9 may be targeted to the spinal cord injury site, brain injury or peripheral nerve injury or a heart after an infarct where there is a chance that fibroblast proliferation subsequently inhibits the normal flow of pulses to the end organ, e.g., muscles, etc. and can be either stimulated by a light pulse stimulus directed to the organ so that it functions e.g., as a pacemaker after a heart attack or Parkinson's disease, etc. to induce an action potential or, e.g., break up an avalanche of action potentials such as in epilepsy.

The inventive method provides solar cells in a minimally invasive procedure into the eye, heart, and/or the central nervous system; the solar cells are not implanted in the body in an invasive procedure. The inventive method provides a plurality of solar cells as discrete individual particles; the solar cells are not connected as a unit and do not have a backing layer or backing material. The inventive method uses solar cells and membrane ion channels and CRISPR/cas9 that may be activated by ambient light; in some embodiments, the method does not use an electrical apparatus and thus does not use photodiodes, stimulating electrodes, or other electrical devices to facilitate or boost the ability of excitable biological cells to normalize or regulate their own polarity.

In one embodiment, the inventive method provides for excitable biological cells to regulate their own polarity; stimulation of the solar cells opsin genes and membrane ion channel genes or their analogues and CRISPR/cas9 used in the invention does not generate an action potential to regulate polarity, but instead facilitates the biological cells themselves to regulate their membrane polarity.

In one embodiment, the inventive method provides nanoparticle, semiconductor particles/opsin genes and other membrane ion channel genes, CRISPR/cas9 and a DNA promoter in combination with therapies to enhance functional recovery of neuronal cells damaged by different etiologies, including genetic disorders, ischemic or vascular damage, and age-related damage or other genetic diseases with neuronal degenerative process are ameliorated and respond with an action potential when stimulated by light or ultrasound.

In one embodiment, the inventive method provides semiconductor particles/opsin genes and CRISPR/cas9 and other membrane ion cannel genes for a sodium ion channel and genes encoding a potassium channel and connexin-43.

In one embodiment, the inventive method provides dendrimer particles/opsin gene(s) with one or multiple CRISPR/cas9 complexes and other membrane ion cannel genes for a sodium ion channel and genes encoding a potassium channel and connexin-43, conjugated with CPP or ACPP and the gene to repair the genetic disease of the cells applied topically through the nasal mucosa, cornea, conjunctiva to be stimulated externally by light and or ultrasound applied locally.

In one embodiment, the inventive method provides dendrimer particles/opsin gene(s) with one or multiple CRISPR/cas9 complexes and other membrane ion channel genes or their analogue genes for a sodium ion channel and genes encoding a potassium channel and connexin-43, conjugated with CPP or ACPP applied by injection to the areas needed, such as eye, brain, spinal cord injury disease, to stimulate also non-excitable cells, such as fibroblasts, glial cells, myofibroblasts, etc. externally by light and or ultrasound applied locally.

In one embodiment, the inventive method provides dendrimer particles/opsin gene(s) with one or multiple CRISPR/cas9 complexes and other membrane ion channel genes for a sodium ion channel and genes encoding a potassium channel and connexin conjugated with CPP or ACPP and the gene(s) to repair the genetic disease of the cells applied topically though CRISPR/cas9 mediated Homology-Independent Targeted Integration (HITI) or Homology Directed Repair (HDR).

In another embodiment, the method provides dendrimer particles conjugated with opsin gene(s) and one or multiple CRISPR/cas9 complexes and other membrane ion cannel genes applied to the nasal mucosa, cornea, conjunctiva, or injected locally or internally in the eye or CSF, to stimulate the cells externally by light or ultrasound applied externally over the nose base by a fiber optic or an ultrasonic transducer to stimulate the excitable cells that are not dividing such as mature neurons or photoreceptors etc.

In one embodiment, the gene acts like oligonucleotide covering the cut gap in the gene created produced by CRISPR/cas9 and does not fill it in, but rather permits the natural healing process to fill the gap in the gene.

In one embodiment, RNAi, CRISPR interference (CRISPRi) turns the genes on or off in a reversible manner.

In one alternative embodiment, one may use the peptide nucleic acid (PNA) to replace CRISPR/cas9 in any of the aforedescribed embodiments to avoid misfiring of the enzyme CRISPR/cas9. Peptide nucleic acid (PNA) is a synthesized polymer that triggers the cell to engage the cell's DNA to correct the mutated gene sequences. PNAs combine a synthetic protein polyamide backbone with specific genomic target site or nucleobases present in DNA and RNA creating PNA/DNA/PNA complex. The PNA molecules are paired with a donor DNA encoding the corrected gene sequence, such as opsin or another gene and conjugated with poly(lactic-co-glycolic acid) (PLGA) nanoparticle and cell penetrating peptides or activatable CPPs and Rock inhibitors to enhance cell penetration and escape from the endosomes and administered either topically to the eye or nasal mucosa, intravitreally, in the cerebrospinal fluid, etc. for treatment of retinal or brain neurodegenerative diseases such as Alzheimer's disease, Parkinson's disease or a degenerative disease of the retina such as retinitis pigmentosa, LCA, diabetic retinopathy.

Also, in one or more alternative embodiments, it should be understood that the CRISPR/cas9 complex can be replaced with other techniques, such as the use of zinc finger (ZFNs), Transcription Activator-like effector-based nucleases (TALENS), etc. or non-homologous end joining can be used as known in the art. In addition, the non-viral particles used in the abovedescribed embodiments can be replaced with viral particles that have the disadvantage that the viral gene and protein is transmitted to the infected cells and can produce an immune response in the patient specifically if repeated administration is needed.

Each reference previously disclosed and disclosed below is expressed incorporated by reference herein in its entirety:
Bakalova et al. Quantum Dot-Conjugated Hybridization Probes for Preliminary Screening of siRNA Sequences. J. Am. Chem. Soc. 127 (2005) 11328-11335.
Biju et al., Chem. Soc. Rev. 39 (2010) 3031
Derfus et al. Targeted Quantum Dot Conjugates for siRNA Delivery. Bioconjugate Chem. 18 (2007) 1391-1396.
Deisseroth, Optogenetics, Nature Methods, published online Dec. 20, 2010, available at http://www.stanford.edu/group/dlab/papers/deisserothnature2010.pdf.
Dixit et al. Quantum Dot Encapsulation in Viral Capsids. Nano Letters, 6 (2006) 1993-1999.
Ebenstein et al. Combining atomic force and fluorescence microscopy for analysis of quantum-dot labeled protein-DNA complexes. J. Molecular Recognition, 22 (2009) 397-402.
Gill et al. Fluorescence Resonance Energy Transfer in CdSe/ZnS-DNA Conjugates: Probing Hybridization and DNA Cleavage. J. Phys. Chem. B, 109 (2005) 23715-23719.
Huang et al. Intermolecular and Intramolecular Quencher Based Quantum Dot Nanoprobes for Multiplexed Detection of Endonuclease Activity and Inhibition. Anal. Chem. 83 (2011) 8913-8918.
Joo et al. Enhanced Real-Time Monitoring of Adeno-Associated Virus Trafficking by Virus-Quantum Dot Conjugates. ACS Nano 5 (2011) 3523-3525.
Lim et al. Specific Nucleic Acid Detection Using Photophysical Properties of Quantum Dot Probes. Anal. Chem. 82 (YEAR) 886-891.
Mossman, Quantum dots track who gets into cell nucleus. Physorg.com, Sep. 2, 2010 http://www.physorg.com/news202628740.html
Sarkar et al. Doped Semiconductor Nanocrystals and Organic Dyes: An Efficient and Greener FRET System J. Phys. Chem. Lett. 1 (2010) 636-640.
Suzuki et al. Quantum Dot FRET Biosensors that Respond to pH, to Proteolytic or Nucleolytic Cleavage, to DNA Synthesis, or to a Multiplexing Combination. J. American Chemical Society 130 (2008) 5720-5725.
Wang et al. Nucleic Acid Conjugated Nanomaterials for Enhanced Molecular Recognition. ACS Nano 3 (2009) 2451-2460.
You et al. Incorporation of quantum dots on virus in polycationic solution. Int. J. Nanomedicine 1 (2006) 59-64.
Audero et al. Sporadic Autonomic Dysregulation and Death Associated with Excessive Serotonin Autoinhibition. Science 321 (2008) 130-133.
De Crespigny et al. Magnetic Resonance Imaging Assessment of Cerebral Hemodynamics During Spreading Depression in Rats. Journal of Cerebral Blood Flow and Metabolism 18 (1998) 1008-1017.
Höhne et al. Acetazolamide prevents hypoxic pulmonary vasoconstriction in conscious dogs. J. Appl. Physiol. 97 (2004) 515-521.
Rio-Portilla et al., REM Sleep POST-EYE Movement Activation, Int' J. Bioelectromagnetism 10 (2008) 192-208.
U.S. Pat. No. 8,324,344

Other variations or embodiments of the invention will also be apparent to one of ordinary skill in the art from the above description. As one example, the inventive technology, in the absence of a viral vector, finds use in plants, animals, and/or cell cultures along with a marker gene, such as GFP, to indicate that the cell, organ, plant, fruit, etc. are genetically modified. As one example, other forms, routes, and sites of administration are contemplated. As another example, the invention may be used in patients who have experienced ocular trauma, retinal degeneration, ischemia, inflammation, etc. As another example, the particles may include sensing devices for qualitative and/or quantitative chemistry or other determinations. For example, the particles may include sensors or other detection means for glucose, oxygen, glycosylated hemoglobin, proteins including but limited to enzymes, pressure, indicators for retinal degenerative disease, etc. Thus, the foregoing embodiments are not to be construed as limiting the scope of this invention.

Any of the features, attributes, or steps of the above described embodiments and variations can be used in combination with any of the other features, attributes, and steps of the above described embodiments and variations as desired.

Although the invention has been shown and described with respect to a certain embodiment or embodiments, it is apparent that this invention can be embodied in many different forms and that many other modifications and variations are possible without departing from the spirit and scope of this invention.

Moreover, while exemplary embodiments have been described herein, one of ordinary skill in the art will readily appreciate that the exemplary embodiments set forth above are merely illustrative in nature and should not be construed as to limit the claims in any manner. Rather, the scope of the invention is defined only by the appended claims and their equivalents, and not, by the preceding description.

The invention claimed is:

1. A method of using a plungerless aspiration and/or injection device, said method comprising the steps of:
providing a plungerless aspiration and/or injection device;
administering to a patient in need thereof, using the plungerless aspiration and/or injection device, a plurality of nanoparticles coated with a biocompatible molecule for cell uptake, the nanoparticles conjugated with at least one gene, an antibody that targets the nanoparticles to a target cell, and a deoxyribonucleic acid (DNA) editing complex forming a complex of coated nanoparticle-gene-DNA editing complex;
delivering the coated nanoparticle-gene-DNA editing complex to the target cell using the nanoparticle of the complex as a carrier of the complex to the target cell without using a viral vector and without using a plasmid vector; and
stimulating the coated nanoparticle-gene-DNA editing complex with one or more energy sources under conditions sufficient to introduce the at least one gene into the target cell and, using the DNA editing complex, to modify a pathology-inducing DNA in the target cell to treat the patient, wherein stimulating the coated nanoparticle-gene-DNA editing complex with the one or more energy sources includes stimulating the nanoparticle of the complex with ultrasound or light.

2. The method according to claim 1, wherein the DNA editing complex is selected from the group consisting of a CRISPR/cas9 complex, a zinc finger nuclease (ZFN) complex, and a transcription activator-like effector-based nucleases (TALENS) complex.

3. The method according to claim 1, wherein the coated nanoparticle-gene-DNA editing complex is further conjugated with one or more Rock inhibitors for reducing inflammation at the site of the target cell, and additionally conjugated with an activatable cell penetrating peptide (ACPP).

4. The method according to claim 1, wherein the nanoparticles are directly attached to DNA containing the at least one gene.

5. The method according to claim 1, further comprising, before administering the coated nanoparticle-gene-DNA editing complex to the patient, administering the coated nanoparticle-gene-DNA editing complex to in vitro cultured patient stem cells.

6. The method according to claim 1, wherein the at least one gene of the coated nanoparticle-gene-DNA editing complex further comprises an opsin-family gene and an ion channel gene, wherein the DNA editing complex comprises a CRISPR/cas9 complex, and wherein the method further comprises the steps of:
administering the coated nanoparticle-gene-DNA editing complex comprising the opsin-family gene, ion channel gene, and the CRISPR/cas9 complex to a non-excitable target cell of the patient; and
stimulating the non-excitable target cell of the patient with light so as to modify cell membrane polarization of the non-excitable target cell and modify the pathology-inducing DNA in the non-excitable target cell to treat the patient.

7. The method according to claim 1, wherein the DNA editing complex comprises a CRISPR/cas9 complex, and wherein gene modification is done using CRISPR/cas9 mediated homology-independent targeted integration (HITI) or homology directed repair (HDR).

8. The method according to claim 1 where the light stimulating the nanoparticle of the complex is delivered by a fiber optic.

9. The method according to claim 1, further comprising the step of:
administering a PNA/DNA/PNA complex to the patient, the PNA/DNA/PNA complex being formed from a peptide nucleic acid (PNA) and a cell penetrating peptide (CPP) or activatable cell penetrating peptide (ACPP), and the PNA/DNA/PNA complex providing therapy for a medical condition of the patient.

10. The method according to claim 1, wherein the plungerless aspiration and/or injection device comprises:
a housing;
a needle portion disposed in the housing, the needle portion configured to be selectively retracted and extended by a user, the needle portion comprising a needle tip configured to be inserted into tissue of a patient for aspiration, injection, and/or implantation; and
a bulb portion disposed in the housing, the bulb portion defining a fluid containing cavity that is fluidly coupled to the needle portion, and the bulb portion being elastically deformable so that the user is able to perform the aspiration, the injection, and/or the implantation on the patient by manipulating the bulb portion.

11. The method according to claim 10, wherein the step of administering the plurality of nanoparticles using the plungerless aspiration and/or injection device further comprises the steps of:
filling the fluid containing cavity of the bulb portion with the plurality of nanoparticles; and
compressing the bulb portion of the plungerless aspiration and/or injection device to inject the plurality of nanoparticles into tissue or a body cavity of the patient.

12. The method according to claim 11, wherein the plurality of nanoparticles comprise a plurality of piezoelectric nanoparticles; and
wherein the step of compressing the bulb portion further comprises compressing the bulb portion to inject the piezoelectric nanoparticles in the vitreous cavity to migrate into a retina of an eye of the patient, or directly into the retina, in the subretinal space, or in the choroid, so that the piezoelectric nanoparticles are able to be stimulated with ultrasound from outside the eye of the patient.

13. The method according to claim 11, further comprising the step of:
filling the fluid containing cavity of the bulb portion with progenitor nerve stem cells or mesenchymal cells in addition to the plurality of nanoparticles; and
wherein the step of compressing the bulb portion further comprises compressing the bulb portion to inject the progenitor nerve stem cells or mesenchymal cells into an eye of the patent so that the progenitor nerve stem cells or mesenchymal cells migrate under a retina of the eye to a central foveal area through retinal intercellular space, and replace retinal cells damaged by age-related macular degeneration or retinitis pigmentosa.

14. The method according to claim 10, wherein the bulb portion is manipulated independently from the retraction and extension of the needle portion such that the needle portion is able to be extended or retracted without deforming the bulb portion.
